(19) United States
(12) Patent Application Publication
(10) Pub. No.: US 2025/0092146 A1
(43) Pub. Date: Mar. 20, 2025

(54) ANTI-CCR8 ANTIBODIES, CONJUGATES, AND USES THEREOF

(71) Applicant: Abilita Bio, Inc., San Diego, CA (US)

(72) Inventors: Alexander Alexandrov, San Diego, CA (US); Karen Chiang, San Diego, CA (US); Le Li, San Diego, CA (US); Roberto Jappelli, Solana Beach, CA (US); Christopher Roth, La Mesa, CA (US); Mauro Mileni, San Diego, CA (US); Michael Hanson, San Marcos, CA (US)

(21) Appl. No.: 18/648,073

(22) Filed: Apr. 26, 2024

Related U.S. Application Data
(60) Provisional application No. 63/498,977, filed on Apr. 28, 2023.

(51) Int. Cl. C07K 16/28; A61P 35/00

(52) U.S. Cl. CPC C07K 16/2866; A61P 35/00; C07K 2317/24; C07K 2317/569

(57) ABSTRACT

Disclosed herein, in certain embodiments, are anti-hCCR8 antibodies, anti-hCCR8 antibody conjugates, and pharmaceutical compositions which comprise the anti-hCCR8 antibodies or conjugates. In some embodiments, also disclosed herein are methods of delivering a payload utilizing an anti-hCCR8 antibody described herein, and methods of treatment with use of an anti-hCCR8 antibody described herein.

### CROSS-REFERENCE

This application claims the benefit of U.S. Provisional Application No. 63/498,977, filed Apr. 28, 2023; which is incorporated herein by reference in their entirety.

### SEQUENCE LISTING

The instant application contains a Sequence Listing which has been filed electronically in XML format and is hereby incorporated by reference in its entirety. Said XML copy, created on Sep. 5, 2024, is named 50247-704_201_SL.xml and is 228,673 bytes in size.

### BACKGROUND OF THE INVENTION

Regulatory T (Treg) cells are a type of T cell that usually regulate the mammalian immune response and prevent the development of autoimmunity. These cells accumulate in certain tumors, establishing a suppressive Tumor Microenvironment (TME), which enables tumors to evade the immune response mediated by effector T cells (Teff). This mechanism of immune avoidance promotes tumor progression and metastasis. Depleting Treg cells in the TME is an attractive approach to reversing immunosuppressive activity and augmenting anti-tumor immunity. Thus, there remains a need for therapies that are specifically able to target and inhibit the activity of Tregs in the tumor microenvironment.

CCR8, a G Protein-Coupled Receptor (GPCR) is differentially upregulated in intratumor Treg cells. However, developing antibodies against GPCR antigens, such as CCR8, is challenging because of their generally low immunogenicity.

### SUMMARY OF THE DISCLOSURE

In some aspects, disclosed herein is an isolated antibody that comprises: a Chemokine receptor 8 (CCR8) binding domain that comprises one or more complementarity-determining regions (CDR) selected from a group consisting of a CDR1 region, a CDR2 region, and a CDR3 region, wherein the CDR1 region comprises an amino acid sequence with at least 70%, 75%, 80% 85%, 90%, 91%, 92%, 93%, 94%, 95%, 96%, 97%, 98%, 99%, or 100% sequence identity to any one of SEQ ID NOs: 2, 3, 11, 12, 47, 54, 57, 58, 112-119, 139-145, or 210-214, the CDR2 region comprises an amino acid sequence with at least 70%, 75%, 80% 85%, 90%, 91%, 92%, 93%, 94%, 95%, 96%, 97%, 98%, 99%, or 100% sequence identity to any one of SEQ ID NOs: 4, 5, 6, 13, 14, 15, 48, 50, 51, 55, 59, 60, 120-125, 146-151, or 216-224, and the CDR3 region comprises an amino acid sequence with at least 70%, 75%, 80% 85%, 90%, 91%, 92%, 93%, 94%, 95%, 96%, 97%, 98%, 99%, or 100% identity with any one of SEQ ID NOs: 8, 9, 17, 49, 52, 53, 56, 61, 62, 63, 64, 65, 66, 67, 68, 69, 126-137, 152-162, 225-229, 243-246. In some cases, the CCR8 binding domain comprises the CDR1 region, the CDR2 region and the CDR3 region. In some cases, the CDR1 region comprises an amino acid sequence with at least 85%, 90%, 95%, 99%, or 100% sequence identity to a sequence selected from SEQ ID NOs: 2, 3, 11, 12, 47, 54, 57, 58, 112-119, 139-145, or 210-214. In some cases, the CDR2 region comprises an amino acid sequence with at least 85%, 90%, 95%, 99%, or 100% sequence identity to a sequence selected from SEQ ID NOs: 4, 5, 6, 13, 14, 15, 48, 50, 51, 55, 59, 60, 120-125, 146-151, or 216-224. In some cases, the CDR3 region comprises an amino acid sequence with at least 85%, 90%, 95%, 99%, or 100% sequence identity to a sequence selected from SEQ ID NOs: 8, 9, 17, 49, 52, 53, 56, 61, 62, 63, 64, 65, 66, 67, 68, 69, 126-137, 152-162, 225-229 or 243-246. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 48, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 13, and the CDR3 region comprises an amino acid sequence with at least 80% identity with any one of SEQ ID NOs: 56. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 6, and the CDR3 region comprises an amino acid sequence with at least 80% identity with any one of SEQ ID NOs: 8. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 13, and the CDR3 region comprises an amino acid sequence with at least 80% identity with any one of SEQ ID NOs: 157. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 48, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 48, and the CDR3 region comprises an amino acid sequence with at least 80% identity with any one of SEQ ID NOs: 52. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 48, and the CDR3 region comprises an amino acid sequence with at least 80% identity with any one of SEQ ID NOs: 53. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 50, and the CDR3 region comprises an amino acid sequence with at least 80% identity with any one of SEQ ID NOs: 52. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 51, and the CDR3 region comprises an amino acid sequence with at least 80% identity with any one of SEQ ID NOs: 243. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 50, and the CDR3 region comprises an amino acid sequence with at least 80% identity with any one of SEQ ID NOs: 53. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 51, and the CDR3 region comprises an amino acid sequence with at least 80% identity with any one of SEQ ID NOs: 244. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 50, and the CDR3 region comprises an amino acid sequence with at least 80% identity with any one of SEQ ID NOs: 245. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 50, and the CDR3 region comprises an amino acid sequence with at least 80% identity with any one of SEQ ID NOs: 246. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 55, and the CDR3 region comprises an amino acid sequence with at least 80% identity with any one of SEQ ID NOs: 56. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 55, and the CDR3 region comprises an amino acid sequence with at least 80% identity with any one of SEQ ID NOs: 61. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 59, and the CDR3 region comprises an amino acid sequence with at least 80% identity with any one of SEQ ID NOs: 62. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 60, and the CDR3 region comprises an amino acid sequence with at least 80% identity with any one of SEQ ID NOs: 63. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 60, and the CDR3 region comprises an amino acid sequence with at least 80% identity with any one of SEQ ID NOs: 64. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 55, and the CDR3 region comprises an amino acid sequence with at least 80% identity with any one of SEQ ID NOs: 65. In some cases, the CDR1 region comprises an amino acid sequence with at least 80% sequence identity to any one of SEQ ID NOs: 57, the CDR2 region comprises an amino acid sequence with at least 80% sequence identity to any one of SEQ ID NOs: 55, and the CDR3 region comprises an amino acid sequence with at least 80% identity with any one of SEQ ID NOs: 66. In some cases, the CDR1 region comprises an amino acid sequence with at least 80% sequence identity to any one of SEQ ID NOs: 58, the CDR2 region comprises an amino acid sequence with at least 80% sequence identity to any one of SEQ ID NOs: 55, and the CDR3 region comprises an amino acid sequence with at least 80% identity with any one of SEQ ID NOs: 67. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 55, and the CDR3 region comprises an amino acid sequence with at least 80% identity with any one of SEQ ID NOs: 68. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 60, and the CDR3 region comprises an amino acid sequence with at least 80% identity with any one of SEQ ID NOs: 69. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 51, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 48, and the CDR3 region comprises an amino acid sequence with at least 80% identity with any one of SEQ ID NOs: 227. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 48, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 210, the CDR2 region comprises an amino acid sequence with at least 80% sequence identity to any one of SEQ ID NOs: 48, and the CDR3 region comprises an amino acid sequence with at least 80% identity with any one of SEQ ID NOs: 226. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 216, and the CDR3 region comprises an amino acid sequence with at least 80% identity with any one of SEQ ID NOs: 227. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 217, and the CDR3 region comprises an amino acid sequence with at least 80% identity with any one of SEQ ID NOs: 227. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 218, and the CDR3 region comprises an amino acid sequence with at least 80% identity with any one of SEQ ID NOs: 227. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 219, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 211, the CDR2 region comprises an amino acid sequence with at least 80% sequence identity to any one of SEQ ID NOs: 220, and the CDR3 region comprises an amino acid sequence with at least 80% identity with any one of SEQ ID NOs: 228. In some cases, the CDR1 region comprises an amino acid sequence with at least 80% sequence identity to any one of SEQ ID NOs: 212, the CDR2 region comprises an amino acid sequence with at least 80% sequence identity to any one of SEQ ID NOs: 221, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 222, and the CDR3 region comprises an amino acid sequence with at least 80% identity with any one of SEQ ID NOs: 227. In some cases, the CDR1 region comprises an amino acid sequence with at least 80% sequence identity to any one of SEQ ID NOs: 47, the CDR2 region comprises an amino acid sequence with at least 80% sequence identity to any one of SEQ ID NOs: 223, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 213, the CDR2 region comprises an amino acid sequence with at least 80% sequence identity to any one of SEQ ID NOs: 51, and the CDR3 region comprises an amino acid sequence with at least 80% identity with any one of SEQ ID NOs: 226. In some cases, the CDR1 region comprises an amino acid sequence with at least 80% sequence identity to any one of SEQ ID NOs: 214, the CDR2 region comprises an amino acid sequence with at least 80% sequence identity to any one of SEQ ID NOs: 48, and the CDR3 region comprises an amino acid sequence with at least 80% identity with any one of SEQ ID NOs: 226. In some cases, the CDR1 region comprises an amino acid sequence with at least 80% sequence identity to any one of SEQ ID NOs: 212, the CDR2 region comprises an amino acid sequence with at least 80% sequence identity to any one of SEQ ID NOs: 221, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 212, the CDR2 region comprises an amino acid sequence with at least 80% sequence identity to any one of SEQ ID NOs: 48, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 212, the CDR2 region comprises an amino acid sequence with at least 80% sequence identity to any one of SEQ ID NOs: 221, and the CDR3 region comprises an amino acid sequence with at least 80% identity with any one of SEQ ID NOs: 226. In some cases, the CDR1 region comprises an amino acid sequence with at least 80% sequence identity to any one of SEQ ID NOs: 212, the CDR2 region comprises an amino acid sequence with at least 80% sequence identity to any one of SEQ ID NOs: 221, and the CDR3 region comprises an amino acid sequence with at least 80% identity with any one of SEQ ID NOs: 49. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 55, and the CDR3 region comprises an amino acid sequence with at least 80% identity with any one of SEQ ID NOs: 56. In some cases, the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 55, and the CDR3 region comprises an amino acid sequence with at least 80% identity with any one of SEQ ID NOs: 229, or the CDR1 region comprises an amino acid sequence with at least 80% sequence identity to any one of SEQ ID NOs: 54, the CDR2 region comprises an amino acid sequence with at least 80% sequence identity to any one of SEQ ID NOs: 224, and the CDR3 region comprises an amino acid sequence with at least 80% identity with any one of SEQ ID NOs: 56.

In some cases, the CCR8 binding domain further comprises one or more framework regions (FR) selected from a group consisting of a FR1 region, a FR2 region, a FR3 region and a FR4 region. In some cases, the isolated antibody comprises the FR1, the FR2, the FR3, and the FR4 region. In some cases, the FR1, the FR2, the FR3 and the FR4 region is human. In some cases, the FR1 region comprises an amino acid sequence with at least 80%, 85%, 90%, 95%, 99%, or 100% sequence identity to any one of SEQ ID NOs: 19, 23, 163, 164, 175, 176, or 177, In some cases, the FR2 region comprises an amino acid sequence with at least 80%, 85%, 90%, 95%, 99%, or 100% sequence identity to any one of SEQ ID NOs: 20, 24, 165, 166, 167, 168, 178, 179, 180, 181, 232, or 233. In some cases, the FR3 region comprises an amino acid sequence with at least 80%, 85%, 90%, 95%, 99%, or 100% sequence identity to any one of SEQ ID NOs: 21, 25, 169, 170, 171, 182, 183, 184, 234, 235, or 236. In some cases, the FR4 region comprises an amino acid sequence with at least 80%, 85%, 90%, 95%, 99%, or 100% sequence identity to any one of SEQ ID NOs: 22, 26, 172, 173, or 185. In some cases, the FR1 region comprises an amino acid sequence with at least 80%, 85%, 90%, 95%, 99%, or 100% sequence identity to SEQ ID NO: 163, the FR2 region comprises an amino acid sequence with at least 80%, 85%, 90%, 95%, 99%, or 100% sequence identity to SEQ ID NO: 165, the FR3 region comprises an amino acid sequence with at least 80%, 85%, 90%, 95%, 99%, or 100% sequence identity to SEQ ID NO: 169, and the FR4 region comprises an amino acid sequence with at least 80%, 85%, 90%, 95%, 99%, or 100% sequence identity to SEQ ID NO: 172. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 166, the FR3 region comprises an amino acid sequence with at least 80%, 85%, 90%, 95%, 99%, or 100% sequence identity to SEQ ID NO: 170, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 167, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 175, the FR2 region comprises an amino acid sequence with at least 80%, 85%, 90%, 95%, 99%, or 100% sequence identity to SEQ ID NO: 178, the FR3 region comprises an amino acid sequence with at least 80%, 85%, 90%, 95%, 99%, or 100% sequence identity to SEQ ID NO: 182, and the FR4 region comprises an amino acid sequence with at least 80%, 85%, 90%, 95%, 99%, or 100% sequence identity to SEQ ID NO: 185. In some cases, the FR1 region comprises an amino acid sequence with at least 80%, 85%, 90%, 95%, 99%, or 100% sequence identity to SEQ ID NO: 176, the FR2 region comprises an amino acid sequence with at least 80%, 85%, 90%, 95%, 99%, or 100% sequence identity to SEQ ID NO: 179, the FR3 region comprises an amino acid sequence with at least 80%, 85%, 90%, 95%, 99%, or 100% sequence identity to SEQ ID NO: 183, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 180, the FR3 region comprises an amino acid sequence with at least 80%, 85%, 90%, 95%, 99%, or 100% sequence identity to SEQ ID NO: 184, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 181, the FR3 region comprises an amino acid sequence with at least 80%, 85%, 90%, 95%, 99%, or 100% sequence identity to SEQ ID NO: 184, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 231, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 232, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 233, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 233, the FR3 region comprises an amino acid sequence with at least 80%, 85%, 90%, 95%, 99%, or 100% sequence identity to SEQ ID NO: 234, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 164, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 233, the FR3 region comprises an amino acid sequence with at least 80%, 85%, 90%, 95%, 99%, or 100% sequence identity to SEQ ID NO: 234, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 234, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 233, the FR3 region comprises an amino acid sequence with at least 80%, 85%, 90%, 95%, 99%, or 100% sequence identity to SEQ ID NO: 234, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 171, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 168, the FR3 region comprises an amino acid sequence with at least 80%, 85%, 90%, 95%, 99%, or 100% sequence identity to SEQ ID NO: 235, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 181, the FR3 region comprises an amino acid sequence with at least 80%, 85%, 90%, 95%, 99%, or 100% sequence identity to SEQ ID NO: 236, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 181, the FR3 region comprises an amino acid sequence with at least 80%, 85%, 90%, 95%, 99%, or 100% sequence identity to SEQ ID NO: 236, and the FR4 region comprises an amino acid sequence with at least 80%, 85%, 90%, 95%, 99%, or 100% sequence identity to SEQ ID NO: 173. In some cases, the FR1 region comprises an amino acid sequence with at least 80%, 85%, 90%, 95%, 99%, or 100% sequence identity to SEQ ID NO: 177, the FR2 region comprises an amino acid sequence with at least 80%, 85%, 90%, 95%, 99%, or 100% sequence identity to SEQ ID NO: 181, the FR3 region comprises an amino acid sequence with at least 80%, 85%, 90%, 95%, 99%, or 100% sequence identity to SEQ ID NO: 184, and the FR4 region comprises an amino acid sequence with at least 80%, 85%, 90%, 95%, 99%, or 100% sequence identity to SEQ ID NO: 173.

In some cases, the FR1 comprises an IgG1, an IgG2, an IgG3 or an IgG4 framework region. In some cases, the FR2 comprises an IgG1, an IgG2, an IgG3 or an IgG4 framework region. In some cases, the FR3 comprises an IgG1, an IgG2, an IgG3 or an IgG4 framework region. In some cases, the CCR8 biding domain comprises an amino acid sequence with at least 80%, 85%, 90%, 95%, 99%, or 100 sequence identity to any one of SEQ ID NOs: 27-30, 70-86, 106-111, or 186-209. In some cases, the CCR8 binding domain comprises an amino acid sequence with at least 80%, 85%, 90%, 95%, 99%, or 100 sequence identity to any one of SEQ ID NOs: 27-30. In some cases, the CCR8 binding domain comprises an amino acid sequence with at least 80%, 85%, 90%, 95%, 99%, or 100 sequence identity to any one of SEQ ID NOs: 70-86. In some cases, the CCR8 binding domain comprises an amino acid sequence with at least 80%, 85%, 90%, 95%, 99%, or 100 sequence identity to any one of SEQ ID NOs: 106-111. In some cases, the CCR8 binding domain comprises an amino acid sequence with at least 80%, 85%, 90%, 95%, 99%, or 100 sequence identity to any one of SEQ ID NOs: 186-209. In some cases, the sequence identity is determined by BLASTp. In some cases, the isolated antibody further comprises a Fc region. In some cases, the Fc region is a human Fc region. In some cases, the Fc region is an IgG1, IgG2, IgG3, IgG4, IgGA1, or an IgGA2 Fc region. In some cases, the isolated antibody further comprises one or more mutations in the Fc region. In some cases, the one or more mutations modulate an effector function of the isolated antibody relative to a corresponding antibody that lacks the at least one mutation. In some cases, the effector function is antibody-dependent cellular cytotoxicity (ADCC), Fc receptor binding, and/or complement-dependent cytotoxicity (CDC). In some cases, the one or more mutations decreases Fc-γ receptor binding of said isolated antibody relative to a corresponding antibody that lacks the at least one mutation. In some cases, the one or more mutations increases cytolytic activity (e.g., increases ADCC activity and/or CDC activity) of said isolated antibody relative to a corresponding antibody that lacks the at least one mutation. In some cases, the one or more mutations is at an amino acid residue selected from the group consisting of L234, L235, G236, 5239, F243, H268, D270, R292, 5298, Y300, V305, K326, A330, 1332, E333, K334, and P396. In some cases, the one or more mutation is a fucosylation. In some cases, the Fc region comprises an amino acid sequence with at least 80%, 85%, 90%, 95%, 99%, or 100% sequence identity to any one of SEQ ID NOs: 34, 44, 238 or 239. In some cases, the Fc region comprises one or more mutations relative to a wild type Fc region that comprises an amino acid sequence of SEQ ID NO: 34, or 237. In some cases, the one or more mutations comprises an amino acid substitution, deletion, insertion, or a combination thereof. In some cases, the one or more mutations comprises: an amino acid substitution of S252, an amino acid substitution of I351, or a combination thereof, relative to a wild type Fc region that comprises an amino acid sequence of SEQ ID NO: 34, numbering according to kabat scheme. In some cases, the amino acid substitution of S252 is a S252D amino acid substitution. In some cases, the amino acid substitution of 1351 is a I351E amino acid substitution. In some cases, the Fc region comprises an amino acid sequence with at least 80%, 85%, 90%, 95%, 99%, or 100% sequence identity to any one of SEQ ID NOs: 34, 44, 238 or 239. In some cases, the isolated antibody comprises an amino acid sequence with at least 80%, 85%, 90%, 95%, 99%, or 100% sequence identity to any one of SEQ ID NOs: 31-33, 45-46 or 87-103.

In some aspects, disclosed herein is an isolated antibody that comprises a Chemokine receptor 8 (CCR8) binding domain, wherein the CCR8 binding domain comprises: an amino acid substitution at G64, an amino acid substitution at L110, an amino acid substitution at 139, an amino acid substitution at G40, an amino acid substitution at R55, an amino acid substitution at R38, an amino acid substitution at R108, an amino acid substitution at R55, an amino acid substitution at A25, an amino acid substitution at N82, an amino acid substitution at G63, an amino acid substitution at S58, an amino acid substitution at G64, an amino acid substitution at S28, an amino acid substitution at T65, an amino acid substitution at A25, an amino acid substitution at R38, an amino acid substitution at S57, an amino acid substitution at S58, an amino acid substitution at S35, an amino acid substitution at R37, an amino acid substitution at R108, an amino acid substitution at R55, an amino acid substitution at N83, an amino acid substitution at A25, an amino acid substitution at S57, an amino acid substitution at S58, an amino acid substitution at S35, an amino acid substitution at G64, an amino acid substitution at R108, an amino acid substitution at R37, an amino acid substitution at R55, an amino acid substitution at R108, an amino acid substitution at N83, an amino acid substitution at A25, an amino acid substitution at R38, an amino acid substitution at S57, an amino acid substitution at N83, an amino acid substitution at R55, an amino acid substitution at S57, an amino acid substitution at R108, an amino acid substitution at G81, an amino acid substitution at S28, or a combination thereof, relative to a wild type CCR8 binding domain that comprises an amino acid sequence of SEQ ID NO: 108, numbering according to IMGT scheme. In some cases, the CCR8 binding domain comprises: the amino acid substitution at G64, the amino acid substitution at L110, the amino acid substitution at 139, the amino acid substitution at G40, and the amino acid substitution at R55, the amino acid substitution at R38, and the amino acid substitution at R108, the amino acid substitution at R55, the amino acid substitution at A25, the amino acid substitution at R55, and the amino acid substitution at N82, the amino acid substitution at G63, and the amino acid substitution at L110, the amino acid substitution at T65, and the amino acid substitution at L110, the amino acid substitution at G64, the amino acid substitution at S28, the amino acid substitution at G64, the amino acid substitution at L110, the amino acid substitution at A25, the amino acid substitution at R38, the amino acid substitution at S57, the amino acid substitution at G64, the amino acid substitution at L110, the amino acid substitution at S58, the amino acid substitution at S35, the amino acid substitution at R37, the amino acid substitution at G64, the amino acid substitution at R108, the amino acid substitution at R37, the amino acid substitution at R55, the amino acid substitution at R108, the amino acid substitution at N83, the amino acid substitution at A25, the amino acid substitution at R38, the amino acid substitution at S57, the amino acid substitution at N83, the amino acid substitution at A25, the amino acid substitution at R38, the amino acid substitution at R55, the amino acid substitution at N83, the amino acid substitution at A25, the amino acid substitution at R38, the amino acid substitution at S57, the amino acid substitution at R108, the amino acid substitution at A25, the amino acid substitution at R28, the amino acid substitution at S57, the amino acid substitution at R108, or the amino acid substitution at A25, the amino acid substitution at R38, the amino acid substitution at S571, the amino acid substitution at G81. In some cases, the amino acid substitution at G64 is G64S or G64T, the amino acid substitution at L110 is L110V or L110Y, the amino acid substitution at 139 is I39Y, the amino acid substitution at G40 is G40S, the amino acid substitution at R55 is R55W, R55H, or R55G, the amino acid substitution at R38 is R38T, the amino acid substitution at R108 is R108I, the amino acid substitution at A25 is A25V, the amino acid substitution at N82 is N82K, the amino acid substitution at G63 is G63I, the amino acid substitution at S58 is S58G, the amino acid substitution at G64 is G64S or G64T, the amino acid substitution at T65 is T651, the amino acid substitution at A25 is A25V, the amino acid substitution at R38 is R38G, an amino acid substitution at S57 is S57I, the amino acid substitution at S58 is S58G, the amino acid substitution at S35 is S35T, the amino acid substitution at R37 is R37V, the amino acid substitution at R108 is R108I, the amino acid substitution at R55 is R55H, the amino acid substitution at N83 is N83K, the amino acid substitution at A25 is A25V, the amino acid substitution at S57 is S57I, the amino acid substitution at R37 is R37V, the amino acid substitution at R108 is R108I, an amino acid substitution at G81 is G81A, and the amino acid substitution at S28 is S28F.

In some aspects, disclosed herein is an isolated antibody that comprises a Chemokine receptor 8 (CCR8) binding domain, wherein the CCR8 binding domain comprises: an amino acid substitution at D81, an amino acid substitution at M108, an amino acid substitution at P65, or a combination thereof, relative to a wild type CCR8 binding domain that comprises an amino acid sequence of SEQ ID NO: 111, numbering according to IMGT scheme. In some cases, the CCR8 binding domain comprises: the amino acid substitution at D81, the amino acid substitution at D81 and M108, or the amino acid substitution at P65. In some cases, the amino acid substitution at D81 is D81G, the amino acid substitution at M108 is M108K, and the amino acid substitution at P65 is P65L.

In any one of the foregoing or related aspects, the isolated antibody comprises a full-length antibody, a chimeric antibody, a single domain antibody, a single-chain antibody (scFv), a heavy-chain-only antibody (HcAb), (e.g., a camelid heavy chain only antibody or a shark heavy-chain-only antibody), or a functional fragment thereof. In some cases, the functional fragment comprises a Fab, Fab′, Fab′-SH, Fv, or a F(ab′)2. In some cases, the isolated antibody comprises a Fab, Fab′, Fab′-SH, Fv, or a F(ab′)2. In some cases, the isolated antibody is a single domain antibody. In some cases, the single domain antibody is a VHH antibody or a VNAR antibody. In some cases, the single domain antibody is a Camelidae VHH. In some cases, the single domain antibody is a llama single domain antibody.

In any one of the foregoing or related aspects, the isolated antibody comprises a first single domain antibody and a second single domain antibody, wherein the first single domain antibody comprises the CCR8 binding domain, and wherein the second single domain antibody comprises an antigen binding domain. In some cases, the antigen binding domain is a CCR8 antigen binding domain. In some cases, the antigen binding domain a cancer antigen binding domain.

In any one of the foregoing or related aspects, the isolated antibody is a bispecific antibody that comprises a first single domain antibody and a second single domain antibody, wherein the first single domain antibody is linked to a second single domain antibody, wherein the first single domain antibody comprises the CCR8 binding domain, and wherein the second single domain antibody comprises an antigen binding domain that binds a target antigen (e.g., a cancer antigen).

In any one of the foregoing or related aspects, the isolated antibody is a bispecific antibody that comprises the CCR8 binding domain and further comprises an antigen binding domain that binds a target antigen (e.g., a cancer antigen).

In any one of the foregoing or related aspects, the isolated antibody is a single chain antibody.

In any one of the foregoing or related aspects, the isolated antibody is humanized, monoclonal, deimmunized, bi-specific, multi-specific, multivalent or a combination thereof. In some cases, the isolated antibody is humanized.

In any one of the foregoing or related aspects, the isolated antibody comprises an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, F(ab′)2, F(ab′)2-scFv2. scFv-KIH, Fab-scFv-Fc, tetravalent HCAb, scDiabody-Fc, diabody-Fc, tandem scFv-Fc, or intrabody.

In any one of the foregoing or related aspects, the isolated antibody specifically binds a CCR8 polypeptide or a portion thereof. In some cases, the isolated antibody specifically binds a human CCR8 polypeptide or a portion thereof. In some cases, the isolated antibody specifically binds an extracellular domain of a CCR8 polypeptide (e.g., a human CCR8 polypeptide) or a portion thereof. In some cases, the isolated antibody specifically binds a transmembrane domain of a CCR8 polypeptide (e.g., a human CCR8) or a portion thereof. In some cases, the isolated antibody specifically binds a cytoplasmic domain of a CCR8 polypeptide (e.g., a human CCR8) or a portion thereof.

In any one of the foregoing or related aspects, the isolated antibody further comprises a payload, wherein the payload is conjugated or coupled to the isolated antibody. In some cases, the payload is selected from a group consisting of a therapeutic agent, a small molecule, a polypeptide, a polynucleotide, an enzyme, a substrate, a cofactor, a fluorescent marker, a chemiluminescent marker, a peptide tag, a magnetic particle, a drug, a therapeutic agent, a siRNA, an antisense oligonucleotide, a toxin, a radionuclide, a binding site for secondary antibodies, a metal binding domain, or a combination thereof.

In any one of the foregoing or related aspects, the isolated antibody is cytolytic. In some cases, the isolated antibody exhibits ADCC activity. In some cases, the isolated antibody is cytolytic to a regulatory T cell (Treg). In some cases, the isolated antibody is cytolytic to a tumor infiltrating regulatory T cell. In some cases, the isolated antibody induces ADCC of a regulatory T cell (Treg) e.g., tumor infiltrating regulatory T cell.

In any one of the foregoing or related aspects, the isolated antibody is capable of (a) enhancing an immune response to a tumor; (b) reducing, depleting, or killing a Treg cells such as tumor infiltrating regulatory T (“Treg”) cells; (c) inducing internalization of a CCR8 polypeptide or the portion thereof in a Treg cell tumor infiltrating regulatory T (“Treg”) cells; (d) binding to the CCR8 polypeptide or the portion thereof (e.g., a human CCR8 polypeptide or a portion thereof); (e) binding to the CCR8 polypeptide or the portion thereof (e.g., human CCR8 polypeptide or a portion thereof) with KD of 10 nM or less as measured by BIACORE™; (f) inducing antibody-dependent cellular cytotoxicity (ADCC) of a cell expressing the CCR8 polypeptide or a portion thereof; or (g) any combination thereof.

In some aspects, disclosed herein is an antibody conjugate comprising the isolated antibody described herein and a payload. In some cases, the payload is a therapeutic agent. In some cases, the payload (e.g., a therapeutic agent) comprises a small molecule, a peptide, a protein, or a polynucleotide (e.g., a DNA, RNA, mRNA). In some cases, the payload (e.g., a therapeutic agent) comprises an immunomodulatory agent, an anti-cancer agent, a cytotoxic agent, a NSAID, a siRNA, an oligonucleotide (e.g., an anti-sense oligonucleotide) a corticosteroid, a dietary supplement such as an antioxidant, or a chemotherapeutic agent. In some cases, the immunomodulatory agent comprises a cytokine, chemokine or an interferon. In some cases, the payload is covalently or non-covalently conjugated to the isolated antibody. In some cases, the payload is conjugated to the isolated antibody by a linker. In some cases, the antibody conjugate comprises: A-(X¹—B)ₙ Formula (I), wherein, A comprises the isolated antibody described herein; B comprises the payload; X¹ comprises a bond or linker; and n is an averaged value selected from 1-12.

In some aspects, disclosed herein is an isolated nucleic acid molecule encoding the isolated antibody described herein; or the antibody conjugate described herein.

In some aspects, disclosed herein is a vector comprising the nucleic acid molecule described herein.

In some aspects, disclosed herein is an in vitro cell, wherein the in vitro cell (a) expresses and/or secretes the isolated antibody described herein, or the antibody described herein; (b) comprises the isolated nucleic acid molecule described herein; and/or (c) comprises the vector described herein.

In some aspects, disclosed herein is a pharmaceutical composition comprising: (a) the isolated antibody described herein, or the antibody conjugate described herein; and (b) a pharmaceutically acceptable carrier, adjuvant or diluent. In some cases, the pharmaceutical composition further comprising at least one additional therapeutic agent. In some cases, the pharmaceutical composition is formulated for administration via a subcutaneous, intravenous, intradermal, intraperitoneal, intramuscular, intracerebroventricular, intracranial, intracelial, or intracerebellar administration route. In some cases, the pharmaceutical composition is in an aqueous or in a lyophilized form. In some cases, the pharmaceutical composition is contained in a delivery device selected from the group consisting of a syringe, a blunt tip syringe, a catheter, and an implantable pump.

In some aspects, disclosed herein is use of the isolated antibody described herein or the antibody conjugate described herein for treating a cancer, or an infection (e.g., a pathogenic infection such as a microbial infection or a viral infection).

In some aspects, disclosed herein is use of the isolated antibody described herein, or the antibody conjugate described herein in the manufacture of a medicament. In some cases, the medicament is for a disease, disorder or condition involving CCR8 polypeptide expressing cells (e.g., Treg cells, e.g., tumor infiltrating Treg cells). In some cases, the medicament is for treatment of a cancer, or an infection (e.g., a pathogenic infection such as a microbial infection or a viral infection).

In some aspects, disclosed herein is use of the antibody conjugate described herein to deliver the payload to a target site. In some cases, the target site is tumor site.

In some aspects, disclosed herein is a fusion protein that comprises the isolated antibody described herein.

In some aspects, disclosed herein is a chimeric antigen receptor (CAR) comprising the isolated antibody described herein.

In some aspects, disclosed herein is a T cell receptor (TCR) comprising the isolated antibody described herein.

In some aspects, disclosed herein is a method of delivering a payload to a target site in a subject, comprising: administering to the subject an effective amount of the antibody conjugate described herein, thereby delivering the payload to the target site. In some cases, the target site is an immune cell or a cell expressing a CCR8 polypeptide or a portion thereof. In some cases, the target site is a tumor site.

In some aspects, disclosed herein is a method of treating a subject in need thereof, comprising: administering to the subject an effective amount of (a) the isolated antibody described herein, (b) the pharmaceutical composition described herein, or (c) the antibody conjugate described herein, thereby treating the subject.

In some aspects, disclosed herein is a method of reducing a level of Treg cells or inhibiting a Treg cell, comprising: administering to a subject in need thereof an effective amount of (a) the isolated antibody described herein, (b) the pharmaceutical composition described herein, or (c) the antibody conjugate described herein; or contacting a population of Treg cells (e.g., a population of infiltrating Treg cells) with an effective amount of (a) the isolated antibody described herein, (b) the pharmaceutical composition described herein, or (c) the antibody conjugate described herein, thereby reducing the level of Treg cells and/or inhibiting the Treg cell.

In some aspects, disclosed herein is a method of enhancing an immune response in a subject in need thereof, comprising: administering to the subject an effective amount of (a) the isolated antibody described herein, (b) the pharmaceutical composition described herein, or (c) the antibody conjugate described herein, thereby treating the subject.

In any one of the foregoing or related aspects, the subject is suffering from, or is at risk of a cancer, or an infection. In some cases, the cancer is a solid cancer. In some cases, the cancer is a hematologic malignancy. In some cases, the cancer is bladder cancer, lung cancer, brain cancer, melanoma, breast cancer, Non-Hodgkin lymphoma, cervical cancer, ovarian cancer, colorectal cancer, pancreatic cancer, esophageal cancer, prostate cancer, kidney cancer, skin cancer, leukemia, thyroid cancer, liver cancer, or uterine cancer. In some cases, the subject is a human. In some cases, the method further comprises administering an effective amount of a second therapeutic agent. In some cases, the administering or contacting results in (a) enhancing an immune response (e.g., immune response to a tumor); (b) reducing, depleting, or killing Treg cells (e.g., tumor infiltrating regulatory T (“Treg”) cells); (c) inducing internalization of a CCR8 polypeptide in Treg cells (e.g., tumor infiltrating regulatory T (“Treg”) cells); (d) binding to the CCR8 polypeptide or a portion thereof (e.g., human CCR8); (e) binding to the CCR8 polypeptide or a portion thereof (e.g., human CCR8) with KD of 10 nM or less as measured by BIACORE™; (f) inducing antibody-dependent cellular cytotoxicity (ADCC) of cells expressing the CCR8 polypeptide or a portion thereof (e.g., Treg cells such tumor infiltrating Treg cells), or (g) any combination thereof. In some cases, the administering is via a subcutaneous, intravenous, intradermal, intraperitoneal, intramuscular, intracerebroventricular, intracranial, intracelial, or intracerebellar administration route. In some cases, the contacting is in vivo or in vitro.

In some aspects, disclosed herein is a method of producing the isolated antibody described herein, the method comprising: (a) culturing the in vitro cell described herein in a medium under conditions permitting expression of a polypeptide encoded by the isolated nucleic acid molecule and assembling of the isolated antibody; and (b) purifying the isolated antibody from the cultured cell or the medium of the in vitro cell.

In some aspects, disclosed herein is a kit comprising: the isolated antibody described herein; the antibody conjugate described herein, the isolated nucleic acid molecule described herein, or the vector described herein. In some cases, the kit further comprises an effective amount of one or more additional therapeutic agents. In some cases, the one or more additional therapeutic agents comprises an anti-cancer agent, a cytotoxic agent, a NSAID, a corticosteroid, a dietary supplement such as an antioxidant, a siRNA, a small molecule, an oligonucleotide, a chemotherapeutic agent, or a combination thereof.

In some aspects, disclosed herein is an isolated antibody that comprises a Chemokine receptor 8 (CCR8) binding domain, wherein the CCR8 binding domain comprises: an amino acid substitution at Q1, an amino acid substitution at Q5, an amino acid substitution at A15, an amino acid substitution at V24, an amino acid substitution at Q49, an amino acid substitution at R50, an amino acid substitution at L52, an amino acid substitution at F70, an amino acid substitution at A83, an amino acid substitution at K95, an amino acid substitution at P96, an amino acid substitution at Q123, or a combination thereof, relative to a wild type CCR8 binding domain that comprises an amino acid sequence of SEQ ID NO: 27, numbering according to IMGT scheme. In some cases, the CCR8 binding domain comprises: the amino acid substitution at Q1, the amino acid substitution at Q5, the amino acid substitution at A15, the amino acid substitution at V24, an amino acid substitution at Q49, the amino acid substitution at R50, the amino acid substitution at L52, the amino acid substitution at F70, the amino acid substitution at A83, the amino acid substitution at K95, the amino acid substitution at P96, and the amino acid substitution at Q123. In some cases, the CCR8 binding domain comprises: the amino acid substitution at Q1, the amino acid substitution at Q5, the amino acid substitution at A15, the amino acid substitution at V24, an amino acid substitution at Q49, the amino acid substitution at A83, the amino acid substitution at K95, and the amino acid substitution at Q123. In some cases, the amino acid substitution at Q1 is Q1V, the amino acid substitution at Q5 is Q5V, the amino acid substitution at A15 is A15P, the amino acid substitution at V24 is V24A, an amino acid substitution at Q49 is Q49G, the amino acid substitution at R50 is R50L, the amino acid substitution at L52 is L52W, the amino acid substitution at F70 is F70S, the amino acid substitution at A83 is A83S, the amino acid substitution at K95 is K95R, the amino acid substitution at P96 is P96A, and the amino acid substitution at Q123 is Q123T.

In some aspects, disclosed herein is an isolated antibody that comprises a Chemokine receptor 8 (CCR8) binding domain, wherein the CCR8 binding domain comprises: an amino acid substitution at Q1, an amino acid substitution at Q5, an amino acid substitution at A15, an amino acid substitution at Q49, an amino acid substitution at R50, an amino acid substitution at L52, an amino acid substitution at R77, an amino acid substitution at A83, an amino acid substitution at R84, an amino acid substitution at S85, an amino acid substitution at A86, an amino acid substitution at L120, an amino acid substitution at Q123, or a combination thereof, relative to a wild type CCR8 binding domain that comprises an amino acid sequence of SEQ ID NO: 28, numbering according to IMGT scheme. In some cases, the CCR8 binding domain comprises: the amino acid substitution at Q1, the amino acid substitution at Q5, the amino acid substitution at A15, the amino acid substitution at Q49, the amino acid substitution at R50, the amino acid substitution at L52, the amino acid substitution at R77, the amino acid substitution at A83, the amino acid substitution at R84, the amino acid substitution at S85, the amino acid substitution at A86, the amino acid substitution at L120, and the amino acid substitution at Q123. In some cases, the CCR8 binding domain comprises: the amino acid substitution at Q1, the amino acid substitution at Q5, the amino acid substitution at A15, the amino acid substitution at Q49, the amino acid substitution at R77, the amino acid substitution at A83, the amino acid substitution at R84, the amino acid substitution at S85, the amino acid substitution at A86, the amino acid substitution at L120, and the amino acid substitution at Q123. In some cases, the amino acid substitution at Q1 is Q1V, the amino acid substitution at Q5 is Q5V, the amino acid substitution at A15 is A15P, the amino acid substitution at Q49 is Q49G, the amino acid substitution at R50 is R50L, the amino acid substitution at L52 is L52W, the amino acid substitution at R77 is R77T, the amino acid substitution at A83 is A83S, the amino acid substitution at R84 is R84K, the amino acid substitution at S85 is S85N, the amino acid substitution at A86 is A86Y, the amino acid substitution at L120 is L120Q, and the amino acid substitution at Q123 is Q123T.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the evaluation by flow cytometry of VHH-1 (SEQ ID NO: 27) VHH-3, (SEQ ID NO: 29) derived from llama immunization with hCCR8 for their specific binding to hCCR8 overexpressed in HEK293 cells and not to human CCR7 (SEQ ID NO: 39).

FIG. 2A shows representative cell binding evaluations on cells expressing hCCR8 of bivalent VHH-Fc antibodies each comprising a fusion of the short hinge of the IgG1 Fc domain (SEQ ID NO: 34), to a unique VHH domain. VHH-1-Fc (SEQ ID NO: 31), VHH-10-Fc (SEQ ID NO: 92), VHH-11-Fc (SEQ ID NO: 93), VHH-12-Fc (SEQ ID NO: 94). BMS-4A19 was evaluated as a comparative positive control. FIG. 2B shows similar representative binding evaluations of VHH-2-Fc (SEQ ID NO: 33), VHH-17-Fc (SEQ ID NO: 99), VHH-18-Fc (SEQ ID NO: 100).

FIGS. 3A-3C illustrates the determination of the binding epitope location using flow cytometry data for two VHH-Fc fusions antibodies, VHH-1-Fc (SEQ ID NO: 31) and VHH-2-Fc (SEQ ID NO: 33) on HEK293T cells transiently transfected with either hCCR8 (SEQ ID NO: 37) or chimeras between hCCR8 (SEQ ID NO: 37) and hCCR1 (SEQ ID NO: 39). FIG. 3A shows conceptual drawings of the two receptor chimera constructs employed in the binding studies are shown to the left and are comprised of different polypeptide regions taken from hCCR8 and hCCR1. FIG. 3B shows binding of selected VHH-Fc antibodies to HEK293 cells expressing Chimera A (SEQ ID NO: 42), indicating interaction with the transmembrane domain of hCCR8. FIG. 3C shows binding of selected VHH-Fc antibodies to HEK293 cells expressing Chimera B (SEQ ID NO: 42), to evaluate interaction only with the hCCR8 N-terminus.

FIGS. 4A-4C shows the evaluation of VHH antibodies formatted as VHH-Fc fusions, by flow cytometry for binding to hCCR8 in the presence or absence of known non-antibody hCCR8 binders. FIG. 4A shows VHH-1-Fc binding in the presence of increasing concentrations of the CCR8 antagonist ML604086 showing competition with antibody binding. FIG. 4B shows that both ML604086 and CCL1 compete for antibody binding to the receptor. FIG. 4C shows cell binding of VHH-1-Fc (SEQ ID NO: 31) VHH-9-Fc (SEQ ID NO: 91), VHH-10-Fc, (SEQ ID NO: 92) and VHH-12-Fc, (SEQ ID NO: 94) to HEK293 cells expressing hCCR8 and complete competition of binding by 10 μM ML604086 (denoted by +ML), while binding of BMS-4A19 is not affected.

FIG. 5 shows that VHH-1-Fc (SEQ ID NO: 31) and VHH-2-Fc (SEQ ID NO: 33) potently inhibit CCL1-induced hCCR8 signaling through the β-arrestin pathway on cells expressing hCCR8, similar to the potent anti-CCR8 binding antibody, BMS-4A19. Inhibition curves represent the inhibition of CCL1 activation of CCR8 by increasing concentrations of the antibodies.

FIG. 6 shows the KABAT and IMGT identification of CDRs and FR regions for the VHH antibodies VHH-1, (SEQ ID NO: 251), VHH-2, (SEQ ID NO: 252), VHH-3 (SEQ ID NO: 253), and VHH-4, (SEQ ID NO: 254).

FIG. 7 shows the activity of VHH-1-Fc-DE (SEQ ID NO: 45) and VHH-2-Fc-DE (SEQ ID NO: 46) in a ADCC reporter bioassay, in which human FcγRIIIa activation on reporter effector cells produce a luminescence signal (RLU) upon engagement with anti-CCR8 VHH-Fc bound to CCR8 expressed on target HuT-78 cells.

FIG. 8A-8B shows the amino acid sequence alignment of VHH-1, (SEQ ID NO: 27) or VHH-2, (SEQ ID NO: 28) to the human germline heavy chain IGHV3-23*04 (SEQ ID NO: 255) and the human J gene joining region IGHJ6*01 (SEQ ID NO: 105) with IMGT sequence numbering. CDR regions are in bold and underlined font and boundaries are defined by IMGT definition. Asterisks denote differences at amino acids between the respective parental llama derived VHH amino acid sequence and the aligned human V and J gene sequences in the framework regions. FIG. 8A shows the alignment of VHH-1 (SEQ ID NO: 27) with the human IGHV3-23*04 (SEQ ID NO: 255) and bold residues indicate where parental llama VHH residues were substitutes to generate humanized versions of VHH-1 (SEQ ID NO: 27) denoted as VHH-1.1 (SEQ ID NO: 106), VHH-1.2 (SEQ ID NO: 107) and VHH-1.3 (SEQ ID NO: 108). FIG. 8B shows the alignment of VHH-2, (SEQ ID NO: 28) with IGHV3-23*04 (SEQ ID NO: 255) and bold residues denote residues that were substituted to generate humanized VHH-1 domains, denoted as VHH-2.1 (SEQ ID NO: 109), VHH-2.2 (SEQ ID NO: 110), and (VHH-2.3) (SEQ ID NO: 111).

FIG. 9A-9B shows evaluations of humanized VHH domains formatted as bivalent Fc fusions to the short hinge of human IgG1 Fc domain (SEQ ID NO: 34) in binding assays to HEK293 cells expressing hCCR8. FIG. 9A shows cells binding curves for VHH-Fc comprising VHH domains from the following group: VHH-1 (SEQ ID NO: 27), VHH-1.2 (SEQ ID NO: 107), and VHH-1.3 (SEQ ID NO: 108). FIG. 9B shows binding of VHH-Fc comprising VHH domains from the following group: VHH-2 (SEQ ID NO: 28), VHH-2.2 (SEQ ID NO: 110), and VHH-2.3 (SEQ ID NO: 111).

FIG. 10 shows of binding curves measured by flow cytometry on HEK293 cells expressing hCCR8 for alanine scanning mutants of the CDR3 region of VHH-1 (SEQ ID NO: 27) in the Fc-fusion antibody format to identify CDR paratope positions that were tolerant of mutations. Mutated positions (IMGT) within the CDR3 region are included in the antibody name.

FIG. 11 shows cell binding analyses of a representative set of humanized VHH-Fc comprising the human IgG Fc (SEQ ID NO: 34) fused to humanized VHH that were optimized by yeast display. Binding to HuT-78 cells measured by flow cytometry and curves are shown for in the absence of presence of 10 μM ML604086 for VHH-Fc fusions of VHH-1.3-13 (SEQ ID NO: 198), VHH-1.3-19 (SEQ ID NO: 204), VHH-1.3-20 (SEQ ID NO: 205), and VHH-1.3-21 (SEQ ID NO: 206), and VHH-1 (SEQ ID NO: 27).

FIG. 12 shows the evaluation of VHH-Fc fusion antibodies in an assay of functional inhibition of CCL1-induced hCCR8 signaling through the β-arrestin pathway. Inhibition curves for VHH-Fc antibodies comprising fusions of VHH to the Fc domain (SEQ ID NO: 34) are shown for the VHH-1 (SEQ ID NO: 27) and the humanized VHH domains, VHH-1.3 (SEQ ID NO: 108), VHH-1.3-19 (SEQ ID NO: 204), and VHH-2.3-2 (SEQ ID NO: 208).

FIG. 13A-13B shows evaluation of ADCC effector function of representative VHH-Fc antibodies as measured through activation of signaling through either the mouse FcγRIV or the human FcγRIIIa Fc on effector cells in response to antibodies bound on HuT-78 target cells. VHH-Fc antibodies each comprise a fusion of a distinct VHH domain to the hinge of the indicated mouse IgG2a Fc (mFc) (SEQ ID NO: 238) or the human IgG1 Fc (SEQ ID NO: 34). FIG. 13A shows the activity of the mFc fusions of VHH-1 (SEQ ID NO: 27), VHH-1.3-7 (SEQ ID NO: 192), VHH-1.3-19, (SEQ ID NO: 204), VHH-1.3-20 (SEQ ID NO: 205), or VHH-1.3-21 (SEQ ID NO: 206) in the absence or the presence (+ML) of the hCCR8 antagonist ML604086 (10 μM). FIG. 13B shows activity of the human Fc fusions of VHH-1.3-17 (SEQ ID NO: 202), VHH-1.3-19, (SEQ ID NO: 204), VHH-1.3-20 (SEQ ID NO: 205), or VHH-1.3-21 (SEQ ID NO: 206).

FIG. 14 shows the growth curves for MC38 tumors in hCCR8 knock-in mice under therapeutic treatment with the VHH-1-mFc antibody SEQ ID NO: 239 or an IgG2a Fc isotype negative control. Points on plots show the average measured tumor volume (10 mice per point) at the indicated number of days after the start of treatment. Measurements are shown as an average of all live mice in the respective treatment group at each point.

### DETAILED DESCRIPTION OF THE DISCLOSURE

The present disclosure addresses the challenge of raising antibodies against the structured extra-cellular domains of CCR8 by employing a strategy that involved immunization of llamas with optimized human CCR8 antigens and then isolating novel hCCR8 antibody binders derived from their specialized antibody repertoire. Camelidae, such as llamas, alpacas, and camels, possess both conventional four-chain IgG antibodies comprised of paired heavy and light chain constituents and unique heavy chain-only antibodies devoid of light chains. These antibodies' two independent and identical antigen binding domains (e.g., VHH) are each formed from a single heavy chain. The specificity and affinity of VHH are comparable to human IgGs, even though the size of a VHH is only about 15 kDa. On average, single domain antibodies such as VHH or heavy chain only antibodies have a longer complementarity determining region 3 (CDR3), which facilitates binding into deeper cavities found on the extracellular surface of GPCRs like hCCR8. In addition, such antibodies can be readily formatted in numerous arrangements to generate biologic drug molecules with optimal properties for use as therapeutics. Disclosed herein, in certain embodiments, are antibodies, and antibody conjugates that specifically bind human CCR8, and pharmaceutical compositions which comprise the antibodies or antibody conjugates that specifically bind human CCR8. In some embodiments, also disclosed herein are methods of delivering a payload utilizing an antibody described herein, and methods of treatment with use of an antibody described herein.

It is to be understood that this application is not limited to particular formulations or process parameters, as these may, of course, vary. It is also to be understood that the terminology used herein is for the purpose of describing particular embodiments only, and is not intended to be limiting. Further, it is understood that a number of methods and materials similar or equivalent to those described herein can be used in the practice of the present inventions.

In accordance with the present application, there may be employed conventional molecular biology, microbiology, and recombinant DNA techniques as explained fully in the art.

The following definitions supplement those in the art and are directed to the current application and are not to be imputed to any related or unrelated case, e.g., to any commonly owned patent or application. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise. Furthermore, use of the term “including” as well as other forms, such as “include”, “includes,” and “included,” is not limiting.

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “or” can be used conjunctively or disjunctively, unless the context specifically refers to a disjunctive use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the invention, and vice versa. Furthermore, compositions of the invention can be used to achieve methods of the invention.

As used herein the term “consisting essentially of” refers to those elements required for a given embodiment. The term permits the presence of elements that do not materially affect the basic and novel or functional characteristic(s) of that embodiment of the invention.

As used herein the term “consisting of” refers to compositions, methods, and respective components thereof as described herein, which are exclusive of any element not recited in that description of the embodiment.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the inventions.

The terms “disease”, “disorder”, or “condition” are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

The term “in need thereof” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

The term “cancer” refers to a disease characterized by the rapid and uncontrolled growth of aberrant cells. Cancer cells can spread locally or through the bloodstream and lymphatic system to other parts of the body.

The term “anti-tumor effect” refers to a biological effect which can be manifested by various means, including but not limited to, e.g., a decrease in tumor volume, a decrease in the number of tumor cells, a decrease in number of regulatory T cells, a decrease in number of immunosuppressive cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place. In some embodiments, the antibodies induce an anti-tumor effect by inducing cytotoxicity. In some embodiments, the antibodies of the disclosure are cytotoxic e.g., cytolytic or cytostatic. The term “cytolytic” as used herein refers to the depletion, elimination and/or the killing of a target cell(s) e.g., cells expressing an antigen or a fragment thereof to which an antibody disclosed herein is capable of binding (e.g., a CCR8 polypeptide). In some embodiments, the cells are immunosuppressive cells. In some embodiments, the cells are Treg cells.

As used herein, a “subject”, “patient”, “individual” and like terms are used interchangeably and refers to a vertebrate, a mammal, a primate, or a human. Mammals include, without limitation, humans, primates, rodents, wild or domesticated animals, including feral animals, farm animals, sport animals, and pets. Primates include, for example, chimpanzees, cynomolgous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, and canine species, e.g., dog, fox, wolf, avian species, e.g., chicken, emu, ostrich, and fish, e.g., trout, catfish and salmon. The terms, “individual,” “patient” and “subject” are used interchangeably herein. A subject can be male or female.

In some embodiments, the subject is a mammal. The mammal can be a human, non-human primate, mouse, rat, dog, cat, horse, or cow, but is not limited to these examples. Mammals other than humans can be advantageously used as subjects that represent animal models of conditions or disorders associated with uncontrolled cell growth (e.g., a cancer). Non-limiting examples include murine tumor models. In addition, the compositions and methods described herein can be used to treat domesticated animals and/or pets. A subject can be one who has been previously diagnosed with or identified as suffering from a cancer. A subject can be one who is diagnosed and currently being treated for, or seeking treatment, monitoring, adjustment or modification of an existing therapeutic treatment, or is at a risk of developing a given disorder (e.g., cancer).

As used herein, the terms “protein”, “peptide” and “polypeptide” are used interchangeably to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric. A polypeptide can have the amino acid sequence of naturally occurring polypeptide from any mammal. Such native sequence polypeptide can be isolated from nature or can be produced by recombinant or synthetic means. In some embodiments, the polypeptide is a “variant”. “Variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide. A “derivative” of a polypeptide is a polypeptide (e.g., an antibody) that has been chemically modified, e.g., via conjugation to another chemical moiety (such as, for example, polyethylene glycol or albumin, e.g., human serum albumin), phosphorylation, and glycosylation.

The terms “increased”, “increase”, or “enhance” are all used herein to generally mean an increase by a statically significant amount; for the avoidance of doubt, the terms “increased”, “increase”, or “enhance”, mean an increase of at least about 2%, 5%, 10%, 15%, 20%, 25%, 30%, 35%, 40%, 45%, 50%, 55%, 60%, 65%, 70%, 75%, 80% 85%, 90%, 95%, 98%, 99%, or up to and including a 100% decrease or up to and including a 100% or more as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms, “decrease”, “reduce”, “reduction”, “lower” or “lowering,” or “inhibit” are all used herein generally to mean a decrease by a statistically significant amount. For example, “decrease”, “reduce”, “reduction”, or “inhibit” means a decrease by at least 10% as compared to a reference level, for example a decrease by at least about 2%, 5%, 10%, 15%, 20%, 25%, 30%, 35%, 40%, 45%, 50%, 55%, 60%, 65%, 70%, 75%, 80% 85%, 90%, 95%, 98%, 99%, or up to and including a 100% decrease (e.g., tumor size after treatment as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. Reduce or inhibit can refer to, for example, the symptoms of the disorder being treated, the presence or size of metastases or micrometastases, the size of the primary tumor, the presence or the size of the dormant tumor. In some embodiments, the reference level is a level in absence of an antibody disclosed herein. In some embodiments, the reference level is a level prior to administering of, contacting, and/or treatment with an antibody disclosed herein. In some embodiments, the reference level is a level in absence of administering of, contacting, and/or treatment with an antibody disclosed herein.

The term “fusion protein” as used herein refers to a polypeptide that comprises an amino acid sequence of an antibody or fragment thereof and an amino acid sequence of a heterologous polypeptide (i.e., an unrelated polypeptide).

As used herein, the term “regulatory T cell,” “T regulatory cell,” Treg,” or “Treg”, used interchangeably herein, refers to a subpopulation of T cells that modulate the immune system, maintain tolerance to self-antigens, and prevent autoimmune disease. Tregs are immunosuppressive and generally suppress or downregulate induction and proliferation of effector T cells. Tregs are known to direct effector T cell lysis, support tolerogenic dendritic cell formation, support M2 macrophage formation, produce immunosuppressive metabolites and cytokines, serve as an IL-2 sink, and to promote neovasculature formation. Though there are many types of Tregs, many Tregs express CD4 and FOXP3, with FOXP3 serving as a marker for Tregs in many cases.

The terms “synthetic polynucleotide,” “synthetic gene” or “synthetic polypeptide,” as used herein, mean that the corresponding polynucleotide sequence or portion thereof, or amino acid sequence or portion thereof, is derived, from a sequence that has been designed, or synthesized de novo, or modified, compared to an equivalent naturally-occurring sequence. Synthetic polynucleotides (antibodies or antigen-binding fragments) or synthetic genes can be prepared by methods known in the art, including but not limited to, the chemical synthesis of nucleic acid or amino acid sequences. Synthetic genes are typically different from naturally-occurring genes, either at the amino acid, or polynucleotide level, (or both) and are typically located within the context of synthetic expression control sequences. Synthetic gene polynucleotide sequences, may not necessarily encode proteins with different amino acids, compared to the natural gene; for example, they can also encompass synthetic polynucleotide sequences that incorporate different codons but which encode the same amino acid (i.e., the nucleotide changes represent silent mutations at the amino acid level).

As used herein, the term “Fc receptor” refers to a polypeptide found on the surface of immune effector cells, which is bound by the Fc region of an antibody. In some aspects, the Fc receptor is an Fcγ receptor. There are three subclasses of Fcγ receptors, FcγRI (CD64), FcγRII (CD32) and FγcRIII (CD16). All four IgG isotypes (IgG1, IgG2, IgG3 and IgG4) bind and activate Fc receptors FcγRI, FcγRIIA and FcγRIIIA. FcγRIIB is an inhibitory receptor, and therefore antibody binding to this receptor does not activate complement and cellular responses. FcγRI is a high affinity receptor that binds to IgG in monomeric form, whereas FcγRIIA and FcγRIIA are low affinity receptors that bind IgG only in multimeric form and have slightly lower affinity. The binding of an antibody to an Fc receptor and/or C1q is governed by specific residues or domains within the Fc regions. Binding also depends on residues located within the hinge region and within the CH2 portion of the antibody. In some aspects, the agonistic and/or therapeutic activity of the antibodies described herein is dependent on binding of the Fc region to the Fc receptor (e.g., FcγR). In some aspects, the agonistic and/or therapeutic activity of the antibodies described herein is enhanced by binding of the Fc region to the Fc receptor (e.g., FcγR).

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a CCR8 epitope is an antibody that binds this epitope with greater affinity, avidity, more readily, and/or with greater duration than it binds to other CCR8 epitopes or non-CCR8 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, the term “Complementarity Determining Regions” (CDRs, i.e., CDR1, CDR2, and CDR3) refers to the amino acid residues of an antibody variable domain the presence of which are necessary for antigen binding. Each variable domain typically has three CDR regions identified as CDR1, CDR2 and CDR3. The CDRs of variable heavy chain can be CDR-H1, CDR-H2 and CDR-H3. The CDRs of variable light chain can be CDR-L1, CDR-L2 and CDR-L3. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat et al., Sequences of Proteins of Immunological Interest, 5th ed. (1991)). Thus, the HVs may be comprised within the corresponding CDRs and references herein to the “hypervariable loops” of VH and VL domains should be interpreted as also encompassing the corresponding CDRs, and vice versa, unless otherwise indicated. With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

With respect to the Chothia numbering system, CDRs within an antibody heavy chain molecule are typically present at amino acid positions 26 to 31, which optionally can include one or two additional amino acids, following 31 (referred to in the Chothia numbering scheme as 31A and 31 B) (CDR1), amino acid positions 52 to 56 (CDR2), and amino acid positions 95 to 102 (CDR3). Using the Chothia numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Chothia numbering system, the actual linear amino acid sequence of the antibody variable domain can contain fewer or additional amino acids due to a shortening or lengthening of a FR and/or CDR and, as such, an amino acid's Chothia number is not necessarily the same as its linear amino acid number.

The more highly conserved regions of variable domains are called the framework region (FR), as defined below. The variable domains of native heavy and light chains each comprise four FRs (FR1, FR2, FR3 and FR4, respectively), largely adopting a [beta]-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817 (1992)); Tramontano et al., J. Mol. Biol, 215: 175-182 (1990)). Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations.

A “variable domain” or an of an antibody refers to the variable domain of the antibody light chain or the variable domain of the antibody heavy chain, either alone or in combination. The variable domains of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Allazikani et al (1997) J. Molec. Biol. 273:927-948)). A CDR may refer to CDRs defined by either approach or by a combination of both approaches. In some embodiments, an antibody of the disclosure comprises a single variable domain (e.g., a heavy chain variable domain, VH).

As used herein, an “antigen binding domain” refers to a binding domain from an antibody or from a non-antibody that can bind to an antigen. An antigen binding domain can be a CCR8 antigen binding domain, a cancer antigen binding domain, or a binding domain that can bind to an antigen (such as a molecule) on an antigen presenting cell. Antigen binding domains can be numbered when there is more than one antigen binding domain in a given conjugate or antibody construct (e.g., first antigen binding domain, second antigen binding domain, third antigen binding domain, etc.). Different antigen binding domains in the same conjugate or construct can target the same antigen or different antigens (e.g., first antigen binding domain that can bind to a tumor antigen, second antigen binding domain that can bind to a molecule on an antigen presenting cell (APC antigen), and third antigen binding domain that can bind to an APC antigen). The term “antigen binding domain” refers to a fragment of an antibody that comprises the area which specifically binds to an epitope, and is complementary to part or all of an antigen. An antigen binding domain may be provided by, for example, one or more antibody variable domains (also called antibody variable regions). In some embodiments, an antigen binding domain comprises a single variable domain, e.g., a heavy chain variable domain (VH). In some embodiments, an antigen binding domain comprises an antibody light chain variable region (VL) and an antibody heavy chain variable region (VH).

A “constant region” of an antibody refers to the constant region of the antibody light chain or the constant region of the antibody heavy chain, either alone or in combination. The constant region does not vary with respect to antigen specificity.

As used herein, the term “heavy chain region” includes amino acid sequences derived from the constant domains of an immunoglobulin heavy chain. A polypeptide comprising a heavy chain region comprises at least one of: a CH1 domain, a hinge (e.g., upper, middle, and/or lower hinge region) domain, a CH2 domain, a CH3 domain, or a variant or fragment thereof. In an embodiment, an antibody or an antigen-binding fragment thereof may comprise the Fc region of an immunoglobulin heavy chain (e.g., a hinge portion, a CH2 domain, and a CH3 domain). In another embodiment, an antibody or an antigen-binding fragment thereof lacks at least a region of a constant domain (e.g., all or part of a CH1 domain). In certain embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, CH2 and CH3 domain sequences from a human immunoglobulin). In certain embodiments, the constituent constant domains of the heavy chain region are from different immunoglobulin molecules. For example, a heavy chain region of a polypeptide may comprise a domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule. That is, the polypeptides of the invention disclosed herein may comprise alterations or modifications to one or more of the heavy chain constant domains (CH1, hinge, CH2 or CH3) and/or to the light chain constant domain (CL). Exemplary modifications include additions, deletions or substitutions of one or more amino acids in one or more domains.

The antibodies or antigen-binding fragment thereof of the present disclosure can comprise a CDR3 region that is a length of at least about 10, 11, 12, 13, 14, 15, 16, 17, 18, 19, 20, 21, 22, 23, 24, 25, 26, 27, 28, 29, or 30 amino acids in length. The antibodies or antigen-binding fragment thereof of the present disclosure can comprise a CDR3 region that is at least about 18 amino acids in length.

As used herein, the term “hinge region” includes the region of a heavy chain molecule that joins the CH1 domain to the CH2 domain. The hinge region can comprise approximately 25 residues and is flexible, thus allowing the two N-terminal antigen binding regions to move independently. Hinge regions can be subdivided into three distinct domains: upper, middle, and lower hinge domains (Roux et al. J. Immunol. 1998 161:4083).

The terms “antibody fragment,” “antigen-binding fragment,” or “antibody binding domain” refer to at least one portion of an antibody, or recombinant variants thereof, that contains the antigen-binding domain, i.e., an antigenic determining variable region of an intact antibody, that is sufficient to confer recognition and specific binding of the antibody fragment to a target, such as an antigen and its defined epitope. Examples of antigen-binding fragments include, but are not limited to, Fab, Fab′, F(ab′)2, and Fv fragments, single-chain (sc)Fv (“scFv”) antibody fragments, linear antibodies, single domain antibodies such as sdAb (either VL or VH), camelid VHH domains, and multi-specific antibodies formed from antibody fragments. In another embodiment, the antibody is a full-length antibody, e.g., an intact IgG1 antibody or other antibody class or isotype as described herein. (See, e.g., Hudson et al. Nat. Med. 9:129-134 (2003); Pluckthiin, The Pharmacology of Monoclonal Antibodies, vol. 113, pp. 269-315 (1994); Hollinger et al., Proc. Natl. Acad. Sci. USA 90: 6444-6448 (1993)). A full-length antibody, intact antibody, or whole antibody is an antibody having a structure substantially similar to a native antibody structure or having heavy chains that contain an Fc region as defined herein. Antibody fragments can be made by various techniques, including but not limited to proteolytic digestion of an intact antibody as well as production by recombinant host cells (e.g., E. coli or phage), as described herein.

As used herein, the term “Fv” is the minimum antibody fragment that contains a complete antigen-recognition and -binding site. This fragment consists of a dimer of one heavy- and one light-chain variable region domain in tight, non-covalent association.

From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Heavy chain variable region” or “VH” with regard to an antibody refers to the fragment of the heavy chain that contains three CDRs, and which further in some embodiments are interposed between flanking stretches known as framework regions, these framework regions are generally more highly conserved than the CDRs and form a scaffold to support the CDRs.

Six hypervariable loops (three loops each from the H and L chain) contribute the amino acid residues for antigen-binding and confer antigen-binding specificity to the antibody. However, even a single variable domain (or half of an Fv comprising only three CDRs specific for an antigen) has the ability to recognize and bind antigen, although at a lower affinity than the entire binding site.

“Framework” or FR residues are those variable domain residues other than the hypervariable region residues.

“Fc region” (region of crystallizable fragments) or “Fc domain” or “Fc” refers to the C-terminal region of an antibody heavy chain that mediates the binding of immunoglobulins to host tissues or factors, including those located in the immune system. In IgG, IgA and IgD antibody isotypes, the Fc region consists of two identical protein fragments derived from the CH2 and CH3 domains of the two heavy chains of the antibody; the Fc regions of IgM and IgE are in each polypeptide chain Contains three heavy chain constant domains (CH domains 2-4). Although the boundaries of the Fc region of an immunoglobulin heavy chain may vary, the Fc region of a human IgG heavy chain is generally defined as the stretch from the amino acid residue at position C226 or P230 of the heavy chain to the carboxy terminus, where this numbering is according to the EU index, as in Same as in Kabat. As used herein, an Fc region can be a native sequence Fc or a variant Fc.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

The term “humanized antibody” used herein refers to antibodies from non-human species whose protein sequences have been modified to increase their similarity to antibody variants produced naturally in humans.

The term “chimeric” antibody refers to an antibody in which a portion of the heavy and/or light chain is derived from a particular source or species, while the remainder of the heavy and/or light chain is derived from a different source or species.

### Antibodies of the Disclosure

The term “antibody” or “immunoglobulin: herein is used in the broadest sense and refers to any polypeptide comprising an antigen-binding domain (e.g., a CCR8 binding domain) with complementarity determining regions (CDR). The term encompasses various antibody structures, including but not limited to monospecific antibodies, multispecific antibodies (for example, bispecific (such as Bi-specific T-cell engagers) and trispecific antibodies), humanized antibodies, chimeric antibodies, human antibodies, single chain antibodies, heavy chain only antibodies (e.g., VHH), llama antibodies, single domain antibodies and nanobodies (e.g., VHH).

The term antibody also includes, but is not limited to, fragments (antibody fragments, or antigen-binding fragments) that are capable of binding to an antigen, such as Fv, single-chain Fv (scFv), Fab, Fab′, di-scFv, sdAb (single domain antibody) and (Fab′)2 (including a chemically linked F(ab′)2). Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. The term antibody also includes, but is not limited to, chimeric antibodies, humanized antibodies, and antibodies of various species such as mouse, human, cynomolgus monkey, etc. Furthermore, for all antibody constructs provided herein, variants having the sequences from other organisms are also contemplated. Thus, if a human version of an antibody is disclosed, one of skill in the art will appreciate how to transform the human sequence-based antibody into a mouse, rat, cat, dog, horse, etc. sequence. Antibody fragments also include either orientation of single chain scFvs, tandem di-scFv, diabodies, tandem tri-sdcFv, minibodies, etc. Antibody fragments also include single domain antibodies (sdAbs), an antibody having a single, monomeric domain, such as a pair of variable domains of heavy chains, without a light chain. An antibody fragment can be referred to as being a specific species in some embodiments (for example, human scFv or a mouse scFv). This denotes the sequences of at least part of the non-CDR regions, rather than the source of the construct.

In some embodiments, an antibody disclosed herein is a full-length antibody. A full-length antibody is a heterotetrameric glycoprotein composed of two identical light (L) chains and two identical heavy (H) chains. IgM antibodies are composed of 5 basic heterotetrameric units and an additional polypeptide called the J chain, containing 10 antigen-binding sites; while IgA antibodies contain 2-5 basic 4-chain units, which can be combined with the J chain Polymerization forms multivalent assemblies. In the case of IgG, the 4-chain unit is typically about 150,000 Daltons. Each light chain is linked to the heavy chain by one covalent disulfide bond, while the two heavy chains are linked to each other by one or more disulfide bonds, the number of which depends on the isotype of the heavy chain. Each heavy and light chain also has regularly spaced intrachain disulfide bridges. Each heavy chain has a variable domain (VH) at the N-terminus, followed by three (for each alpha and gamma chain, CH1, CH2 and CH3) and four (for the mu and epsilon isoforms, CH1, CH2, CH3 and CH4) constant domains (CH) and a hinge region (Hinge) between the CH1 and CH2 domains. Each light chain has a variable domain (VL) at the N-terminus followed by a constant domain (CL) at its other end. VL is aligned with VH and CL is aligned with the first constant domain (CH1) of the heavy chain. Particular amino acid residues are thought to form the interface between the light and heavy chain variable domains. The paired VH and VL together form an antigen binding site. For the structure and properties of different classes of antibodies, see, eg, Basic and Clinical Immunology, Eighth Edition, eds. by Daniel P. Sties, Abba I. Terr and Tristram G. Parsolw, Appleton & Lange, Norwalk, CT, 1994, pp. 71 and 6 chapter. Light chains from any vertebrate species can be assigned to one of two distinct types called kappa and lambda based on their constant domain amino acid sequences. Immunoglobulins can be assigned to different classes or isotypes based on their heavy chain constant domain (CH) amino acid sequences. There are five classes of immunoglobulins: IgA, IgD, IgE, IgG, and IgM, with heavy chains called alpha, delta, epsilon, gamma, and mu, respectively. The gamma and alpha classes can be further divided into subclasses based on relatively minor differences in CH sequence and function, e.g., humans express the following subclasses: IgG1, IgG2A, IgG2B, IgG3, IgG4, IgA1 and IgA2. The antibodies of the present disclosure can also be referred to as an anti-hCCR8 antibody.

The present disclosure provides antibodies that specifically bind human CCR8. In some embodiments, the antibodies or antigen binding fragment thereof induce lysis of cells expressing CCR8 (e.g., Treg cells) and/or cancer cells. Lysis can be induced by any mechanism, such as by mediating an effector function, such as C1q binding and complement dependent cytotoxicity (CDC); Fc receptor binding; antibody-dependent cell-mediated cytotoxicity (ADCC); phagocytosis, or direct induction of cell apoptosis.

In some embodiments, an antibody disclosed herein, is modified to have an increase in at least one effector function as compared to the non-modified parent antibody. Effector functions are biological activities attributable to the Fc region of an antibody, which vary with the antibody isotype. These effector functions can also be interchangeably called “Fc receptor mediated effector functions” In some embodiments, an antibody disclosed herein comprises an Fc region. In some embodiments, an antibody disclosed herein comprises at least one mutation in the Fc region. In some embodiment, the at least one mutation in the Fc region modulates (e.g., increases) an effector function of the antibody relative to a corresponding antibody lacking the at least one mutation. Examples of effector functions include, but not limited to: C1q binding and complement dependent cytotoxicity (CDC); Fc receptor binding; antibody-dependent cell-mediated cytotoxicity (ADCC); phagocytosis. For example, an antibody disclosed herein can be glycoengineered to have at least one increase in effector function as compared to the non-glycoengineered parent. Antibody-dependent cellular cytotoxicity (ADCC) is the result of the formation of a complex between the Fab portion of the antibody with the antigen on the cell surface and binding of the Fc portion to the Fc receptors (FcγRs), on effector cells. The increase in an effector function can be an increased binding affinity to an Fc receptor, increased ADCC; increased cell mediated immunity; increased binding to cytotoxic CD8 T cells; increased binding to NK cells; increased binding to macrophages; increased binding to polymorphonuclear cells; increased binding to monocytes; increased binding to macrophages; increased binding to large granular lymphocytes; increased binding to granulocytes; direct signaling inducing apoptosis; increased dendritic cell maturation; or increased T cell priming.

In some embodiments, an antibody disclosed herein comprises an antigen binding domain (CCR8 binding domain) that comprises one, two or three complementarity-determining regions (CDR) selected from a group consisting of a CDR1 region, a CDR2 region, and a CDR3 region, wherein the CDR1 region comprises an amino acid sequence with at least 70%, 75% 80%, 85%, 90%, 91%, 92%, 93%, 94%, 95%, 96%, 97%, 98%, 99%, or sequence identity to any one of SEQ ID NOs: 1, 2, 3, 10, 11, 12, 47, 54, 57, 58, 112-119, 139-145, or 210-214, wherein the CDR2 region comprises an amino acid sequence with at least 70%, 75% 80%, 85%, 90%, 91%, 92%, 93%, 94%, 95%, 96%, 97%, 98%, 99%, or sequence identity to any one of SEQ ID NOs: 4, 5, 6, 13, 14, 15, 48, 50, 51, 55, 59, 60, 120-125, 146-151, or 215-224, and wherein the CDR3 region comprises an amino acid sequence with at least 70%, 75% 80%, 85%, 90%, 91%, 92%, 93%, 94%, 95%, 96%, 97%, 98%, 99%, or identity with any one of SEQ ID NOs: 7, 8, 9, 16, 17, 18, 49, 52, 53, 56, 61, 62, 63, 64, 65, 66, 67, 68, 69, 126-137, 152-162, 225-229, or 243-246.

In some embodiments, an antibody disclosed herein comprises an amino acid sequence with at least about 50%, 60%, 70%, 75%, 80% 85%, 90%, 91%, 92%, 93%, 94%, 95%, 96%, 97%, 98%, 99%, or 100% sequence identity to the amino acid sequence of any one of SEQ ID NO: 27-30, 70-86, 106-111, 186-209, 31-33, 45-46 or 87-103. In some embodiments, the antibody comprises an antigen binding domain (e.g., a CCR8 binding domain that comprises an amino acid sequence with at least about 50%, 60%, 70%, 75%, 80% 85%, 90%, 91%, 92%, 93%, 94%, 95%, 96%, 97%, 98%, 99%, or 100% sequence identity to the amino acid sequence of any one of SEQ ID NO: 27-30, 70-86, 106-111, or 186-209. In some embodiments, an antibody disclosed herein further comprises an Fc region. In some embodiments, an antibody disclosed herein is a single domain antibody (e.g. VHH), wherein said antibody comprises an amino acid sequence with at least about 50%, 60%, 70%, 75%, 80% 85%, 90%, 91%, 92%, 93%, 94%, 95%, 96%, 97%, 98%, 99%, or 100% sequence identity to the amino acid sequence of any one of SEQ ID NO: 27-30, 70-86, 106-111, 186-209, 31-33, 45-46 or 87-103. In some embodiments, an antibody of the present disclosure comprises a fusion of a single domain antibody and a Fc region, wherein the antibody comprises an amino acid sequence with at least about 50%, 60%, 70%, 75%, 80% 85%, 90%, 91%, 92%, 93%, 94%, 95%, 96%, 97%, 98%, 99%, or 100% sequence identity to the amino acid sequence of any one of SEQ ID NO: 31-33, 45-46 or 87-103. In some embodiments, the Fc region comprises an amino acid sequence with at least about 50%, 60%, 70%, 75%, 80% 85%, 90%, 91%, 92%, 93%, 94%, 95%, 96%, 97%, 98%, 99%, or 100% sequence identity to SEQ ID NO: 34, 44, 238 or 239.

In one aspect, an antibody disclosed herein having at least 50%, 60%, 70%, 75%, 80% 85%, 90%, 91%, 92%, 93%, 94%, 95%, 96%, 97%, 98%, 99%, or 100% sequence identity to any one of SEQ ID NO: 27-30, 70-86, 106-111, 186-209, 31-33, 45-46 or 87-103, comprises substitutions (e.g., conservative substitutions), insertions, or deletions relative to the reference sequence, but an antibody comprising that sequence retains the ability to bind to antigen (e.g., CCR8). In some embodiments, a total of 1 to 11 amino acids have been substituted, inserted and/or deleted in the amino acid sequence of any one of SEQ ID NO: 27-30, 70-86, 106-111, 186-209, 31-33, 45-46 or 87-103. In some embodiments, substitutions, insertions, or deletions occur in regions outside the CDRs (e.g., in the FRs). Optionally, the antibody comprises an amino acid sequence with at least about 50%, 60%, 70%, 75%, 80% 85%, 90%, 91%, 92%, 93%, 94%, 95%, 96%, 97%, 98%, 99%, or 100% sequence identity to the amino acid sequence of any one of SEQ ID NO: 27-30, 70-86, 106-111, 186-209, 31-33, 45-46 or 87-103, including post-translational modifications of that sequence.

In some embodiments, a total of 1 to 11 amino acids have been substituted, inserted and/or deleted in any one of the amino acid sequence of any one of SEQ ID NO: 27-30, 70-86, 106-111, 186-209, 31-33, 45-46 or 87-103.

In one aspect, an antibody disclosed herein comprises a CDR1, CDR2, and a CDR3, wherein the selected CDR1, CDR2, and CDR3 are paired according to Table 2. In one aspect, an antibody disclosed herein comprises a CDR1, CDR2, and a CDR3, wherein the selected CDR1, CDR2, and CDR3 are paired according to Table 2, and further comprises a FR1 region, FR2 region, FR3 region, and FR4 region, wherein a FR1 region, FR2 region, FR3 region, and FR4 region are paired according to table 14. In one aspect, an antibody disclosed herein comprises a CDR1, CDR2, and a CDR3, wherein the selected CDR1, CDR2, and CDR3 are paired according to Table 4. In one aspect, an antibody disclosed herein comprises a CDR1, CDR2, and a CDR3, wherein the selected CDR1, CDR2, and CDR3 are paired according to Table 4, and further comprises a FR1 region, FR2 region, FR3 region, and FR4 region, wherein a FR1 region, FR2 region, FR3 region, and FR4 region are paired according to table 14. In one aspect, an antibody disclosed herein comprises a CDR1, CDR2, and a CDR3, wherein the selected CDR1, CDR2, and CDR3 are paired according to Table 14. In one aspect, an antibody disclosed herein comprises a CDR1, CDR2, and a CDR3, wherein the selected CDR1, CDR2, and CDR3 are paired according to Table 14, and further comprises a FR1 region, FR2 region, FR3 region, and FR4 region, wherein a FR1 region, FR2 region, FR3 region, and FR4 region are paired according to table 14.

In some embodiments, an antibody disclosed herein comprises one, two, three, or four framework regions (FR) selected from group consisting of FR1 region, FR2 region, FR3 region and FR4 region. In some embodiments, an antibody disclosed herein comprises a FR1 region, FR2 region, FR3 region, and FR4 region, wherein a FR1 region, FR2 region, FR3 region, and FR4 region are paired according to table 14. In some embodiments, the FR1 region comprises an amino acid sequence having at least 50%, 60%, 70%, 75%, 80% 85%, 90%, 91%, 92%, 93%, 94%, 95%, 96%, 97%, 98%, 99%, or 100% sequence identity to any one of SEQ ID NO: 19, 23, 163, 164, 175, 176, 177, or 230. In some embodiments, the FR1 region having at least 50%, 60%, 70%, 75%, 80% 85%, 90%, 91%, 92%, 93%, 94%, 95%, 96%, 97%, 98%, 99%, or 100% sequence identity to any one of SEQ ID NO: 19, 23, 163, 164, 175, 176, 177, or 230, comprises substitutions (e.g., conservative substitutions), insertions, or deletions relative to the reference sequence, wherein the antibody retains the ability to bind to antigen (e.g., CCR8). In some embodiments, a total of 1 to 11 amino acids have been substituted, inserted and/or deleted in the amino acid sequence of any one of SEQ ID NOs 19, 23, 163, 164, 175, 176, 177, or 230.

In some embodiments, the FR2 region comprises an amino acid sequence having at least 50%, 60%, 70%, 75%, 80% 85%, 90%, 91%, 92%, 93%, 94%, 95%, 96%, 97%, 98%, 99%, or 100% sequence identity to any one of SEQ ID NO: 20, 24, 165, 166, 167, 168, 178, 179, 180, 181, 232, or 233. In some embodiments, the FR2 region having at least 50%, 60%, 70%, 75%, 80% 85%, 90%, 91%, 92%, 93%, 94%, 95%, 96%, 97%, 98%, 99%, or 100% sequence identity to any one of SEQ ID NO: 20, 24, 165, 166, 167, 168, 178, 179, 180, 181, 232, or 233, comprises substitutions (e.g., conservative substitutions), insertions, or deletions relative to the reference sequence, wherein the antibody retains the ability to bind to antigen (e.g., CCR8). In some embodiments, a total of 1 to 11 amino acids have been substituted, inserted and/or deleted in the amino acid sequence of any one of SEQ ID NOs 20, 24, 165, 166, 167, 168, 178, 179, 180, 181, 232, or 233.

In some embodiments, the FR3 region comprises an amino acid sequence having at least 50%, 60%, 70%, 75%, 80% 85%, 90%, 91%, 92%, 93%, 94%, 95%, 96%, 97%, 98%, 99%, or 100% sequence identity to any one of SEQ ID NO: 21, 25, 169, 170, 171, 182, 183, 184, 234, 235, or 236. In some embodiments, the FR3 region having at least 50%, 60%, 70%, 75%, 80% 85%, 90%, 91%, 92%, 93%, 94%, 95%, 96%, 97%, 98%, 99%, or 100% sequence identity to any one of SEQ ID NO: 21, 25, 169, 170, 171, 182, 183, 184, 234, 235, or 236, comprises substitutions (e.g., conservative substitutions), insertions, or deletions relative to the reference sequence, wherein the antibody retains the ability to bind to antigen (e.g., CCR8). In some embodiments, a total of 1 to 11 amino acids have been substituted, inserted and/or deleted in the amino acid sequence of any one of SEQ ID NOs 21, 25, 169, 170, 171, 182, 183, 184, 234, 235, or 236.

In some embodiments, the FR4 region comprises an amino acid sequence having at least 50%, 60%, 70%, 75%, 80% 85%, 90%, 91%, 92%, 93%, 94%, 95%, 96%, 97%, 98%, 99%, or 100% sequence identity to any one of SEQ ID NO: 22, 26, 172, 173, 185. In some embodiments, the FR4 region having at least 50%, 60%, 70%, 75%, 80% 85%, 90%, 91%, 92%, 93%, 94%, 95%, 96%, 97%, 98%, 99%, or 100% sequence identity to any one of SEQ ID NO: 22, 26, 172, 173, 185 or 237, comprises substitutions (e.g., conservative substitutions), insertions, or deletions relative to the reference sequence, wherein the antibody retains the ability to bind to antigen (e.g., CCR8). In some embodiments, a total of 1 to 11 amino acids have been substituted, inserted and/or deleted in the amino acid sequence of any one of SEQ ID No. 22, 26, 172, 173, 185 or 237.

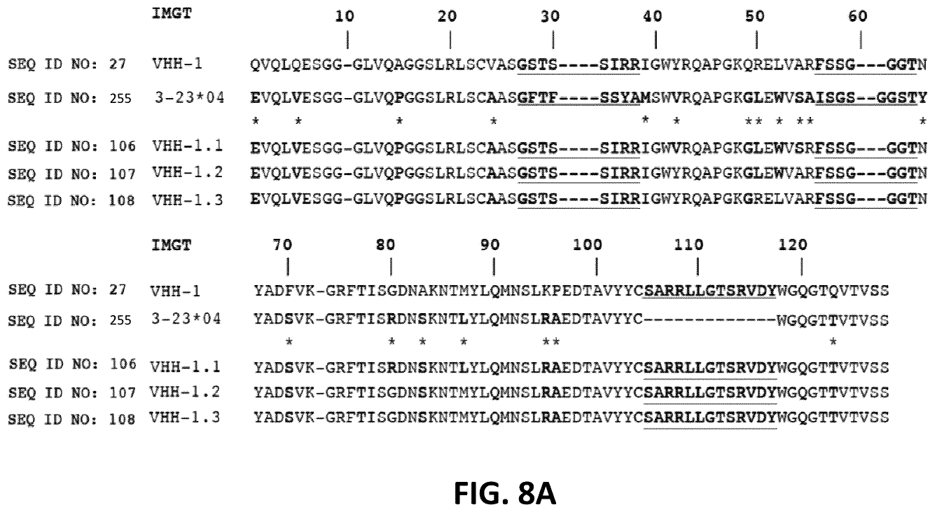

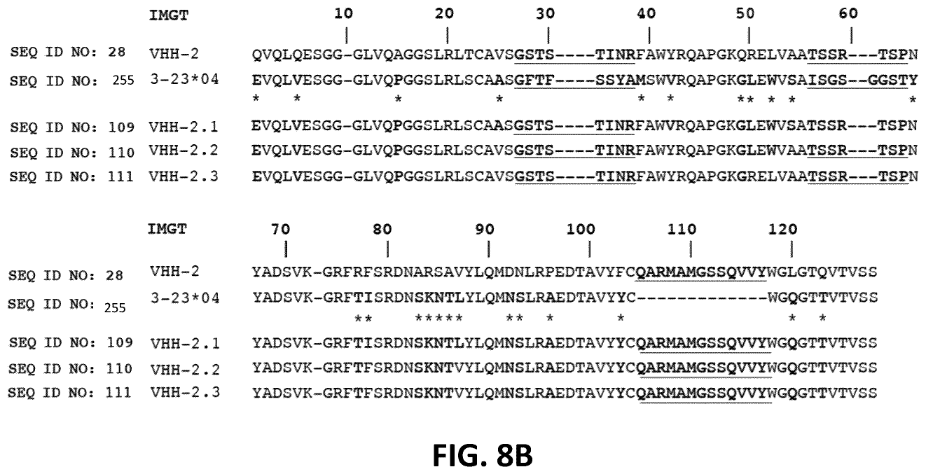

In some embodiments, an antibody of the disclosure, comprises a VHH antibody corresponding to SEQ ID NOs: 27-30. FIG. 8A-8B presents a schematic representation of the amino acid sequences of example VHH including SEQ ID NOs: 27, 28, wherein CDRs are identified using the IMGT method (underlined).

Accordingly, using the IMGT method, the CDRs as identified within each VHH antibody sequence (FIG. 8A-8B), as defined above, correspond to those in Table 1 below:

TABLE 1
Exemplary amino acid sequences
of complementarity-determining
regions (CDR) as identified
within a VHH domain antibody.

| SEQ | Description | |
|---|---|---|
| ID | of the | Amino Acid |
| NO: | Sequence | Sequence |
| 47 | CDR-1 | GSTSSIRR |
| 2 | CDR-1 | GSTSSIRL |
| 3 | CDR-1 | ESTSSIRR |
| 4 | CDR-2 | SSDGSTN |
| 5 | CDR-2 | SRGGGTN |
| 6 | CDR-2 | SSGGGTN |
| 49 | CDR-3 | SARRLLGTSRVDY |
| 8 | CDR-3 | GARRLLGTSRVDY |
| 9 | CDR-3 | SARRLRGTSRVDN |
| 54 | CDR-1 | GSTSTINR |
| 11 | CDR-1 | GSTTTINR |
| 12 | CDR-1 | GSTSTSNR |
| 13 | CDR-2 | SSRTSPN |
| 14 | CDR-2 | SSRTGPN |
| 15 | CDR-2 | SARTGPN |
| 56 | CDR-3 | QARMAMGSSQVVY |
| 17 | CDR-3 | QARIAMGSSQVVY |
| 157 | CDR-3 | QARMAMGASQVVY |

In some embodiments, an antibody of the disclosure comprises at least one, preferably at least two and most preferably three CDR(s) selected from the group consisting of SEQ ID NOs: 1, 2, 3, 10, 11, 12, 47, 54, 57, 58, 112-119, 139-145, 210-214, 4, 5, 6, 13, 14, 15, 48, 50, 51, 55, 59, 60, 120-125, 146-151, 215-224, 7, 8, 9, 16, 17, 18, 49, 52, 53, 56, 61, 62, 63, 64, 65, 66, 67, 68, 69, 126-137, 152-162, 243-246 and 225-229, or at least one, preferably at least two and most preferably three amino acid sequence(s) having at least 80% amino acid identity to said CDR sequences, or at least one, preferably at least two and most preferably three amino acid sequence(s) having 3, 2, or 1 amino acid sequence difference with said CDR sequences.

In some embodiments, an antibody of the disclosure comprises a CDR3 that comprises an amino acid sequence selected from any one of SEQ ID Nos: 7, 8, 9, 16, 17, 18, 49, 52, 53, 56, 61, 62, 63, 64, 65, 66, 67, 68, 69, 126-137, 152-162, 225-229 or 243-246, (c) amino acid sequences having at least 80% amino acid sequence identity with any one of SEQ ID NO: 7, 8, 9, 16, 17, 18, 49, 52, 53, 56, 61, 62, 63, 64, 65, 66, 67, 68, 69, 126-137, 152-162, 225-229 or 243-246 and (d) amino acid sequences having 3, 2 or 1 amino acid different from any one of SEQ ID NO: 7, 8, 9, 16, 17, 18, 49, 52, 53, 56, 61, 62, 63, 64, 65, 66, 67, 68, 69, 126-137, 152-162, 225-229, or 243-246. More preferably, CDR3 corresponds to SEQ ID NO: 7, 8, 9, 16, 17, 18, 49, 52, 53, 56, 61, 62, 63, 64, 65, 66, 67, 68, 69, 126-137, 152-162, 225-229, or 243-246.

In some embodiments, an antibody of the disclosure comprises a CDR1 that comprises an amino acid sequence selected from any one of SEQ ID Nos: 1, 2, 3, 10, 11, 12, 47, 54, 57, 58, 112-119, 139-145, or 210-214, (c) amino acid sequences having at least 80% amino acid sequence identity with any one of SEQ ID NO: 1, 2, 3, 10, 11, 12, 47, 54, 57, 58, 112-119, 139-145, or 210-214 and (d) amino acid sequences having 3, 2 or 1 amino acid different from any one of SEQ ID NO: 1, 2, 3, 10, 11, 12, 47, 54, 57, 58, 112-119, 139-145, or 210-214. More preferably, CDR1 corresponds to SEQ ID NO: 1, 2, 3, 10, 11, 12, 47, 54, 57, 58, 112-119, 139-145, or 210-214.

In some embodiments, an antibody of the disclosure comprises a CDR2 that comprises an amino acid sequence selected from any one of SEQ ID Nos: 4, 5, 6, 13, 14, 15, 48, 50, 51, 55, 59, 60, 120-125, 146-151, or 215-224, (c) amino acid sequences having at least 80% amino acid sequence identity with any one of SEQ ID NO: 4, 5, 6, 13, 14, 15, 48, 50, 51, 55, 59, 60, 120-125, 146-151, or 215-224 and (d) amino acid sequences having 3, 2 or 1 amino acid different from any one of SEQ ID NO: 4, 5, 6, 13, 14, 15, 48, 50, 51, 55, 59, 60, 120-125, 146-151, or 215-224. More preferably, CDR2 corresponds to SEQ ID NO: 4, 5, 6, 13, 14, 15, 48, 50, 51, 55, 59, 60, 120-125, 146-151, or 215-224.

In a particular embodiment, the present disclosure provides hCCR8 binding antibody comprising a combination of the CDR1, CDR2, and CDR3 sequences as described herein, including the allowable variation described for these CDR regions.

In some embodiments, an antibody of the disclosure comprises a sequence containing three CDRs having the sequence of SEQ ID NO: 1, 4 and 7 or SEQ ID NO: 3, 6 and 9, or comprises three CDRs having the sequence of SEQ ID NO: 12, 15 and 18 or SEQ ID NO: 10, 13 and 16.

In some embodiments, an antibody of the disclosure, further comprises a sequence with at least 85, 90, 95, 98 or 99% sequence identified to at least one framework region (FR) of a single-domain antibody moiety described herein. In another embodiment of the invention, the single-domain antibody moiety, as detailed above, further comprises an amino acid sequence having at least 85, 90, 95, 98, or 99% sequence identity to the four framework regions (FR) of a single-domain antibody moiety described herein. It is understood that the method used for determining the FRs of said single-domain antibody moiety is the same as that used for identifying the CDRs.

Accordingly, using the IMGT method, the FRs as identified within the single-domain antibody moiety, as defined above, corresponds to:

SEQ ID NO: 163:
QVQLQESGGGLVQAGGSLRLSCVAS
SEQ ID NO: 20:
IGWYRQAPGKQRELVARF
SEQ ID NO: 21:
YADFVKGRFTISGDNAKNTMYLQMNSLKPEDTAVYYC
SEQ ID NO: 172:
WGQGTQVTVSS
SEQ ID NO: 175:
QVQLQESGGGLVQAGGSLRLTCAVS
SEQ ID NO: 24:
FAWYRQAPGKQRELVAAT
SEQ ID NO: 25:
YADSVKGRFRFSRDNARSAVYLQMDNLRPEDTAVYFC
SEQ ID NO: 185:
WGLGTQVTVSS

In some embodiments, an antibody of the disclosure comprises at least one, preferably at least two, more preferably at least three, and most preferably four amino acid sequences having at least 85%, preferably 90%, more preferably 95% sequence identity to any one of sequences selected from the group consisting of SEQ ID NOs: 19, 23, 163, 164, 175, 176, 177, 230, 20, 24, 165, 166, 167, 168, 178, 179, 180, 181, 232, 233, 21, 25, 169, 170, 171, 182, 183, 184, 234, 235, 236, 22, 26, 172, 173, 185 or 237.

In some embodiments, an antibody of the disclosure comprises four framework regions (FRs) according to the format FR1-CDR1-FR2-CDR2-FR3-CDR3-FR4, wherein FR1 has at least 85%, preferably 90%, more preferably 95% sequence identity to the sequence of SEQ ID NO: 19, 23, 163, 164, 175, 176, 177, or 230, FR2 has at least 85%, preferably 90%, more preferably 95% sequence identity to the sequence of SEQ ID NO: 20, 24, 165, 166, 167, 168, 178, 179, 180, 181, 232, or 233, FR3 has at least 85%, preferably 90%, more preferably 95% sequence identity to the sequence of SEQ ID NO: 21, 25, 169, 170, 171, 182, 183, 184, 234, 235, or 236 and FR4 has at least 85%, preferably 90%, more preferably 95% sequence identity to the sequence of SEQ ID NO: 22 or 26.

In some embodiments, an antibody of the disclosure comprises an amino acid sequence selected from the group consisting of SEQ ID NO: 22, 26, 172, 173, 185 or 237.

SEQ ID NO: 27
QVQLQESGGGLVQAGGSLRLSCVASGSTSSIRRIGWYRQAPGKQRELVAR
FSSGGGTNYADFVKGRFTISGDNAKNTMYLQMNSLKPEDTAVYYCSARRL
LGTSRVDYWGQGTQVTVSS
SEQ ID NO: 28
QVQLQESGGGLVQAGGSLRLTCAVSGSTSTINRFAWYRQAPGKQRELVAA
TSSRTSPNYADSVKGRFRFSRDNARSAVYLQMDNLRPEDTAVYFCQARMA
MGSSQVVYWGLGTQVTVSS
SEQ ID NO: 29
QVQLQESGGGLVQAGGSLRLSCVASGSTSSIRRIGWYRQAPGKQRELVAR
FSSGGGTNYADFVKGRFTISGDNAKNTMYLQMNSLKPEDTAVYYCGARRL
LGTSRVDYWGQGTQVTVSS
SEQ ID NO: 30:
QVQLQESGGGLVQAGGSLRLTCAVSGSTSTINRFAWYRQAPGKQRELVAA
TSSRTSPNYADSVKGRFRFSRDNARSAVYLQMDNLRPEDTAVYFCQARMA
MGASQVVYWGLGTQVTVSS

TABLE 2
Exemplary VHH antibodies and corresponding groups
of complementarity determining regions.

| | SEQ ID NOs | | | |
|---|---|---|---|---|
| | VHH SEQ | CDR 3 SEQ | CDR 2 SEQ | CDR 1 SEQ |
| VHH Name | ID NO: | ID NO: | ID NO: | ID NO: |
| VHH-1 | 27 | 49 | 48 | 47 |
| VHH-2 | 28 | 56 | 55 | 54 |
| VHH-3 | 29 | 8 | 6 | 47 |
| VHH-4 | 30 | 157 | 13 | 54 |

In some embodiments, an antibody described supra is a full-length antibody. In other embodiments, the antibody is a binding fragment thereof. In some cases, the antibody is a humanized antibody or binding fragment thereof, a chimeric antibody or binding fragment thereof, a monoclonal antibody or binding fragment thereof, a multi-specific antibody or binding fragment thereof, or a bispecific antibody or binding fragment thereof. In some cases, the antibody is monovalent Fab′, divalent Fab₂, F(ab)′₃ fragments, single-chain variable fragment (scFv), bis-scFv, (scFv)₂, diabody, minibody, nanobody, triabody, tetrabody, disulfide stabilized Fv protein (“dsFv”), single-domain antibody (sdAb), Ig NAR, camelid antibody or binding fragment thereof, or a chemically modified derivative thereof.

In some embodiments, the antibody is a multi-specific antibody. In some cases, the multi-specific antibody comprises two or more target antigen binding domains in which each of the two or more antigen binding domains binds specifically to an antigen, and the two or more antigens are different. In some cases, the multi-specific antibody comprises antigen binding domains that specifically bind to three or more different antigens, four or more different antigens, or five or more different antigens.

In some embodiments, an antibody disclosed herein is a bispecific antibody. In some cases, the bispecific antibody or binding fragment includes a Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), FcAAdp, XmAb, Azymetric, Bispecific Engagement by Antibodies based on the T-cell receptor (BEAT), Bispecific T-cell Engager (BiTE), Biclonics, Fab-scFv-Fc, Two-in-one/Dual Action Fab (DAF), FinomAb, scFv-Fc-(Fab)-fusion, Dock-aNd-Lock (DNL), Adaptir (previously SCORPION), Tandem diAbody (TandAb), Dual-affinity-ReTargeting (DART), or nanobody.

In some instances, the bispecific antibody is a trifunctional antibody or a bispecific mini-antibody. In some cases, the bispecific antibody is a trifunctional antibody. In some instances, the trifunctional antibody is a full-length monoclonal antibody comprising binding sites for two different antigens.

In some cases, the bispecific antibody is a bispecific mini-antibody. In some instances, the bispecific mini-antibody comprises divalent Fab₂, F(ab)′₃ fragments, bis-scFv, (scFv)₂, diabody, minibody, triabody, tetrabody or a bi-specific T-cell engager (BiTE). In some embodiments, the bi-specific T-cell engager is a fusion protein that contains two single-chain variable fragments (scFvs) in which the two scFvs target epitopes of two different antigens.

In some instances, an antibody disclosed herein is a trispecific antibody. In some instances, the trispecific antibody comprises F(ab)′₃ fragments or a triabody. In some embodiments, the anti-hCCR8 antibody is a trispecific antibody as described in Dimas, et al., “Development of a trispecific antibody designed to simultaneously and efficiently target three different antigens on tumor cells,” Mol. Pharmaceutics, 12(9): 3490-3501 (2015).

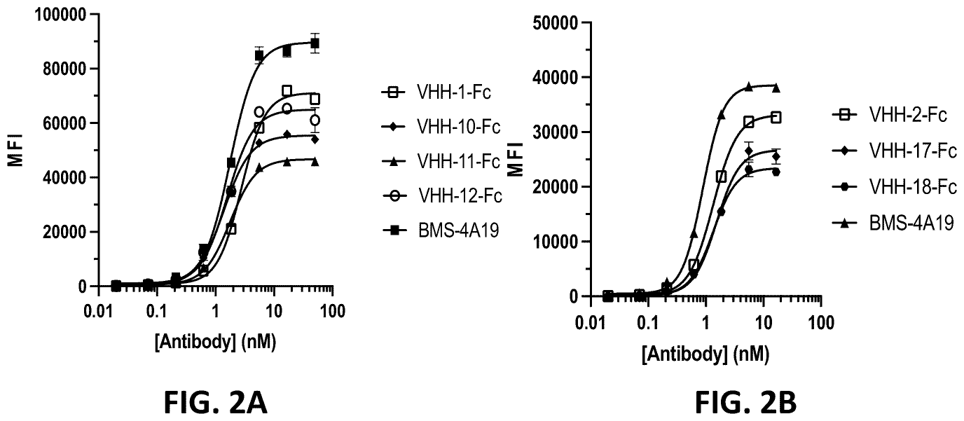

In some instances, an antobody disclosed herein comprises an antibody format illustrated in FIG. 2 of Brinkmann and Kontermann, “The making of bispecific antibodies,” MABS 9(2): 182-212 (2017).

In some embodiments, an antibody described herein comprises an IgG framework, an IgA framework, an IgE framework, or an IgM framework. In some instances, the antibody comprises an IgG framework (e.g., IgG1, IgG2, IgG3, or IgG4). In some cases, the antibody comprises an IgG1 framework. In some cases, the antibody comprises an IgG2 (e.g., an IgG2a or IgG2b) framework. In some cases, the antibody comprises an IgG2a framework. In some cases, the antibody comprises an IgG2b framework. In some cases, the antibody comprises an IgG3 framework. In some cases, the 8 antibody comprises an IgG4 framework.

In one particular embodiment, an antibody described herein is comprised of the fusion of a single domain antibody (e.g., VHH) to the hinge region of an lgG Fc domain derived from lgG1, lgG2, lgG3 and lgG4 antibody. In some embodiments, the Fc region is an lgG Fc domain (SEQ ID NO: 34) derived from a human lgG1 antibody. In some embodiments, the Fc region is an lgG Fc domain derived from a short hinge variant of a human lgG1 antibody. In some embodiments, an antibody described herein is a genetically engineered polypeptide that comprises at least one single domain antibody molecule (e.g., VHH) joined to an IgG1 Fc domain by a direct bond. In some embodiments, the Fc region comprises the amino acid sequence of SEQ ID NO: 34 or an amino acid sequence having at least 80% sequence identity, 85% sequence identity or 90% sequence identity to SEQ ID NO: 34.

TABLE 3
Exemplary amino acid sequence of the disclosure

| SEQ | | | |
|---|---|---|---|
| ID | | Description of | |
| NO: | Name | Sequence | Amino Acid Sequence |
| 31 | VHH-1-Fc | Amino acid | QVQLQESGGGLVQAGGSLRLSCVASGSTSSIRRI |
| | | sequence of | GWYRQAPGKQRELVARFSSGGGTNYADFVKGRFT |
| | | VHH-Fc fusion | ISGDNAKNTMYLQMNSLKPEDTAVYYCSARRLLG |
| | | antibody | TSRVDYWGQGTQVTVSSDKTHTCPPCPAPELLGG |
| | | | PSVFLFPPKPKDTLMISRTPEVTCVVVDVSHEDP |
| | | | EVKFNWYVDGVEVHNAKTKPREEQYNSTYRVVSV |
| | | | LTVLHQDWLNGKEYKCKVSNKALPAPIEKTISKA |
| | | | KGQPREPQVYTLPPSRDELTKNQVSLTCLVKGFY |
| | | | PSDIAVEWESNGQPENNYKTTPPVLDSDGSFFLY |
| | | | SKLTVDKSRWQQGNVFSCSVMHEALHNHYTQKSL |
| | | | SLSPGK |
| 32 | VHH-3-Fc | Amino acid | QVQLQESGGGLVQAGGSLRLSCVASGSTSSIRRI |
| | | sequence of | GWYRQAPGKQRELVARFSSGGGTNYADFVKGRFT |
| | | VHH-Fc fusion | ISGDNAKNTMYLQMNSLKPEDTAVYYCGARRLLG |
| | | antibody | TSRVDYWGQGTQVTVSSDKTHTCPPCPAPELLGG |
| | | | PSVFLFPPKPKDTLMISRTPEVTCVVVDVSHEDP |
| | | | EVKFNWYVDGVEVHNAKTKPREEQYNSTYRVVSV |
| | | | LTVLHQDWLNGKEYKCKVSNKALPAPIEKTISKA |
| | | | KGQPREPQVYTLPPSRDELTKNQVSLTCLVKGFY |
| | | | PSDIAVEWESNGQPENNYKTTPPVLDSDGSFFLY |
| | | | SKLTVDKSRWQQGNVFSCSVMHEALHNHYTQKSL |
| | | | SLSPGK |
| 33 | VHH-2-Fc | Amino acid | QVQLQESGGGLVQAGGSLRLTCAVSGSTSTINRF |
| | | sequence of | AWYRQAPGKQRELVAATSSRTSPNYADSVKGRFR |
| | | VHH-Fc fusion | FSRDNARSAVYLQMDNLRPEDTAVYFCQARMAMG |
| | | antibody | SSQVVYWGLGTQVTVSSDKTHTCPPCPAPELLGG |
| | | | PSVFLFPPKPKDTLMISRTPEVTCVVVDVSHEDP |
| | | | EVKFNWYVDGVEVHNAKTKPREEQYNSTYRVVSV |
| | | | LTVLHQDWLNGKEYKCKVSNKALPAPIEKTISKA |
| | | | KGQPREPQVYTLPPSRDELTKNQVSLTCLVKGFY |
| | | | PSDIAVEWESNGQPENNYKTTPPVLDSDGSFFLY |
| | | | SKLTVDKSRWQQGNVFSCSVMHEALHNHYTQKSL |
| | | | SLSPGK |
| 34 | IgG1 Fc | Fc region amino | DKTHTCPPCPAPELLGGPSVFLFPPKPKDTLMIS |
| | domain | acid sequence of | RTPEVTCVVVDVSHEDPEVKFNWYVDGVEVHNAK |
| | | human IgG1 | TKPREEQYNSTYRVVSVLTVLHQDWLNGKEYKCK |
| | | antibody | VSNKALPAPIEKTISKAKGQPREPQVYTLPPSRD |
| | | including the | ELTKNQVSLTCLVKGFYPSDIAVEWESNGQPENN |
| | | hinge domain | YKTTPPVLDSDGSFFLYSKLTVDKSRWQQGNVFS |
| | | | CSVMHEALHNHYTQKSLSLSPGK |
| 35 | BMS-4A19 | Amino acid | QVQLVQSGAEVKKPGASVKVSCKASGYTFTDSEM |
| | | sequence BMS- | HWVRQATGQGLEWMGAIQPETGGTAYNQKFKARV |
| | | 4A19 antibody | TMTRDTSISTAYMELSSLRSEDTAVYYCARRRRN |
| | | complete heavy | FDYWGQGTLVTVSSASTKGPSVFPLAPSSKSTSG |
| | | chain | GTAALGCLVKDYFPEPVTVSWNSGALTSGVHTFP |
| | | | AVLQSSGLYSLSSVVTVPSSSLGTQTYICNVNHK |
| | | | PSNTKDKRVEPKSCDKTHTCPPCPAPELLGGPSV |
| | | | FLFPPKPKDTLMISRTPEVTCVVVDVSHEDPEVK |
| | | | FNWYVDGVEVHNAKTKPREEQYNSTYRVVSVLTV |
| | | | LHQDWLNGKEYKCKVSNKALPAPIEKTISKAKGQ |
| | | | PREPQVYTLPPSREEMTKNQVSLTCLVKGFYPSD |
| | | | IAVEWESNGQPENNYKTTPPVLDSDGSFFLYSKL |
| | | | TVDKSRWQQGNVFSCSVMHEALHNHYTQKSLSLS |
| | | | PG |
| 36 | BMS-4A19 | Amino acid | DIVMTQTPLSLSVTPGQPASISCRSSQSLFHSSG |
| | | sequence BMS- | NTYLHWYLQKPGQPPQLLIYKVSNRFSGVPDRFS |
| | | 4A19 antibody | GSGSGTDFTLKISRVEAEDVGVYYCSQSTHVPFT |
| | | complete light | FGQGTKLEIKRTVAAPSVFIFPPSDEQLKSGTAS |
| | | chain | VVCLLNNFYPREAKVQWKVDNALQSGNSQESVTE |
| | | | QDSKDSTYSLSSTLTLSKADYEKHKVYACEVTHQ |
| | | | GLSSPVTKSFNRGEC |
| 37 | hCCR8 | Amino acid | MDYTLDLSVTTVTDYYYPDIFSSPCDAELIQTNG |
| | | sequence of | KLLLAVFYCLLFVFSLLGNSLVILVLVVCKKLRS |
| | | hCCR8 | ITDVYLLNLALSDLLFVFSFPFQTYYLLDQWVFG |
| | | | TVMCKVVSGFYYIGFYSSMFFITLMSVDRYLAVV |
| | | | HAVYALKVRTIRMGTTLCLAVWLTAIMATIPLLV |
| | | | FYQVASEDGVLQCYSFYNQQTLKWKIFTNFKMNI |
| | | | LGLLIPFTIFMFCYIKILHQLKRCQNHNKTKAIR |
| | | | LVLIVVIASLLFWVPFNVVLFLTSLHSMHILDGC |
| | | | SISQQLTYATHVTEIISFTHCCVNPVIYAFVGEK |
| | | | FKKHLSEIFQKSCSQIFNYLGRQMPRESCEKSSS |
| | | | CQQHSSRSSSVDYIL |
| 38 | hCCR8-NT | Amino acid | MDYTLDLSVTTVTDYYYPDIFSS |
| | | sequence of | |
| | | hCCR8 N- | |
| | | terminus region, | |
| | | AA 1-23 | |
| 39 | hCCR1 | Amino acid | METPNTTEDYDTTTEFDYGDATPCQKVNERAFGA |
| | | sequence of | QLLPPLYSLVFVIGLVGNILVVLVLVQYKRLKNM |
| | | human CCR1 | TSIYLLNLAISDLLFLFTLPFWIDYKLKDDWVFG |
| | | | DAMCKILSGFYYTGLYSEIFFIILLTIDRYLAIV |
| | | | HAVFALRARTVTFGVITSIIIWALAILASMPGLY |
| | | | FSKTQWEFTHHTCSLHFPHESLREWKLFQALKLN |
| | | | LFGLVLPLLVMIICYTGIIKILLRRPNEKKSKAV |
| | | | RLIFVIMIIFFLFWTPYNLTILISVFQDFLFTHE |
| | | | CEQSRHLDLAVQVTEVIAYTHCCVNPVIYAFVGE |
| | | | RFRKYLRQLFHRRVAVHLVKWLPFLSVDRLERVS |
| | | | STSPSTGEHELSAGF |
| 40 | hCCR1-NT | Amino acid | METPNTTEDYDTTTEFDYGDAT |
| | | sequence of | |
| | | hCCR8 N- | |
| | | terminus region, | |
| | | AA 1-22 | |
| 41 | hCCR7 | Amino acid | MDLGKPMKSVLVVALLVIFQVCLCQDEVTDDYIG |
| | | sequence of | DNTTVDYTLFESLCSKKDVRNFKAWFLPIMYSII |
| | | human CCR7 | CFVGLLGNGLVVLTYIYFKRLKTMTDTYLLNLAV |
| | | | ADILFLLTLPFWAYSAAKSWVFGVHFCKLIFAIY |
| | | | KMSFFSGMLLLLCISIDRYVAIVQAVSAHRHRAR |
| | | | VLLISKLSCVGIWILATVLSIPELLYSDLQRSSS |
| | | | EQAMRCSLITEHVEAFITIQVAQMVIGFLVPLLA |
| | | | MSFCYLVIIRTLLQARNFERNKAIKVIIAVVVVF |
| | | | IVFQLPYNGVVLAQTVANFNITSSTCELSKQLNI |
| | | | AYDVTYSLACVRCCVNPFLYAFIGVKFRNDLFKL |
| | | | FKDLGCLSQEQLRQWSSCRHIRRSSMSVEAETTT |
| | | | TFSP |
| 42 | Chimera A | Amino acid | METPNTTEDYDTTTEFDYGDATPDIFSSPCDAEL |
| | | sequence of | IQTNGKLLLAVFYCLLFVFSLLGNSLVILVLVVC |
| | | hCCR8 chimeric | KKLRSITDVYLLNLALSDLLFVFSFPFQTYYLLD |
| | | construct where | QWVFGTVMCKVVSGFYYIGFYSSMFFITLMSVDR |
| | | the N-terminus | YLAVVHAVYALKVRTIRMGTTLCLAVWLTAIMAT |
| | | (amino acids | IPLLVFYQVASEDGVLQCYSFYNQQTLKWKIFTN |
| | | 1-23) is | FKMNILGLLIPFTIFMFCYIKILHQLKRCQNHNK |
| | | replaced by | TKAIRLVLIVVIASLLFWVPFNVVLFLTSLHSMH |
| | | amino acids | ILDGCSISQQLTYATHVTEIISFTHCCVNPVIYA |
| | | 1-22 from hCCR1 | FVGEKFKKHLSEIFQKSCSQIFNYLGRQMPRESC |
| | | | EKSSSCQQHSSRSSSVDYIL |
| 43 | Chimera B | Amino acid | METPNTTEDYDTTTEFDYGDATPCQKVNERAFGA |
| | | sequence of | QLLPPLYSLVFVIGLVGNILVVLVLVQYKRLKNM |
| | | hCCR1 chimeric | TSIYLLNLAISDLLFLFTLPFWIDYKLKDDWVFG |
| | | construct where | DAMCKILSGFYYTGLYSEIFFIILLTIDRYLAIV |
| | | the N-terminus | HAVFALRARTVTFGVITSIIIWALAILASMPGLY |
| | | (amino acids | FSKTQWEFTHHTCSLHFPHESLREWKLFQALKLN |
| | | 1-22) is | LFGLVLPLLVMIICYTGIIKILLRRPNEKKSKAV |
| | | replaced by | RLIFVIMIIFFLFWTPYNLTILISVFQDFLFTHE |
| | | amino acids | CEQSRHLDLAVQVTEVIAYTHCCVNPVIYAFVGE |
| | | 1-23 from hCCR8 | RFRKYLRQLFHRRVAVHLVKWLPFLSVDRLERVS |
| | | | STSPSTGEHELSAGF |
| 44 | IgG1 Fc- | Amino acid | DKTHTCPPCPAPELLGGPDVFLFPPKPKDTLMIS |
| | DE | sequence of the | RTPEVTCVVVDVSHEDPEVKFNWYVDGVEVHNAK |
| | | human IgG1 Fc | TKPREEQYNSTYRVVSVLTVLHQDWLNGKEYKCK |
| | | domain with | VSNKALPAPEEKTISKAKGQPREPQVYTLPPSRD |
| | | S252D and | ELTKNQVSLTCLVKGFYPSDIAVEWESNGQPENN |
| | | I351E (DE) | YKTTPPVLDSDGSFFLYSKLTVDKSRWQQGNVFS |
| | | ADCC | CSVMHEALHNHYTQKSLSLSPGK |
| | | enhancing | |
| | | mutations | |
| | | (Kabat | |
| | | definition) | |
| 45 | VHH-1-Fc- | Amino acid | QVQLQESGGGLVQAGGSLRLSCVASGSTSSIRRI |
| | DE | sequence of | GWYRQAPGKQRELVARFSSGGGTNYADFVKGRFT |
| | | VHH-Fc | ISGDNAKNTMYLQMNSLKPEDTAVYYCSARRLLG |
| | | antibody with | TSRVDYWGQGTQVTVSSDKTHTCPPCPAPELLGG |
| | | S252D and | PDVFLFPPKPKDTLMISRTPEVTCVVVDVSHEDP |
| | | I351E (DE) | EVKFNWYVDGVEVHNAKTKPREEQYNSTYRVVSV |
| | | ADCC | LTVLHQDWLNGKEYKCKVSNKALPAPEEKTISKA |
| | | enhancing | KGQPREPQVYTLPPSRDELTKNQVSLTCLVKGFY |
| | | mutations | PSDIAVEWESNGQPENNYKTTPPVLDSDGSFFLY |
| | | in the human Fc | SKLTVDKSRWQQGNVFSCSVMHEALHNHYTQKSL |
| | | domain | SLSPGK |
| | | (Kabat | |
| | | definition) | |
| 46 | VHH-2-Fc- | Amino acid | QVQLQESGGGLVQAGGSLRLTCAVSGSTSTINRF |
| | DE | sequence of | AWYRQAPGKQRELVAATSSRTSPNYADSVKGRFR |
| | | VHH-Fc | FSRDNARSAVYLQMDNLRPEDTAVYFCQARMAMG |
| | | antibody with | SSQVVYWGLGTQVTVSSDKTHTCPPCPAPELLGG |
| | | S252D and | PDVFLFPPKPKDTLMISRTPEVTCVVVDVSHEDP |
| | | I351E (DE) | EVKFNWYVDGVEVHNAKTKPREEQYNSTYRVVSV |
| | | ADCC | LTVLHQDWLNGKEYKCKVSNKALPAPEEKTISKA |
| | | enhancing | KGQPREPQVYTLPPSRDELTKNQVSLTCLVKGFY |
| | | mutations | PSDIAVEWESNGQPENNYKTTPPVLDSDGSFFLY |
| | | in the human Fc | SKLTVDKSRWQQGNVFSCSVMHEALHNHYTQKSL |
| | | domain (Kabat | SLSPGK |
| | | definition) | |

In some embodiments, the antibody is an isolated antibody. In some embodiments, the antibody is a recombinant antibody. In some embodiments, the antibody is an engineered antibody.

In some cases, an antibody described herein comprises one or more mutations in a framework region, e.g., in the CH1 domain, CH2 domain, CH3 domain, hinge region, or a combination thereof.

In some embodiments the antibody described herein comprises a humanized antibody. As for full-size antibodies, single variable domains such as VHHs can be subjected to sequence optimization, such as humanization, i.e., increase the degree of sequence identity with the closest human germline sequence, and other optimization techniques, such as to improve physicochemical or other properties of the binders. In particular, humanized immunoglobulin single variable domains, such as VHHs and may be single-domain antibodies in which at least one single amino acid residue is present (and in particular, at least one framework residue) that is and/or that corresponds to a humanizing substitution (as defined further herein).

Humanized antibodies e.g., single-domain antibodies, e.g., VHHs, may have several advantages, such as a reduced immunogenicity, compared to the corresponding naturally occurring antibody e.g., singly domain antibodies, e.g., VHHs. By humanized, it is meant that certain amino acids are mutated so that immunogenicity upon administration in human patients is minor or non-existent. The humanizing substitutions should be chosen such that the resulting humanized amino acid sequence and/or VHH still retains the favorable properties of the VHH antibody, such as the antigen-binding capacity.

In some embodiments, the antibody e.g., a VHH antibody as described herein, is an optimized humanized antibody e.g., single domain antibody e.g., a VHH antibody. In some embodiments, the antibody disclosed herein e.g., a single domain antibody e.g., a VHH antibody has been optimized by introducing mutations, e.g., amino acid substitutions, for example at any one of positions of 1, 5, 15, 24, 42, 49, 50, 52, 54, 55, 70, 80, 83, 87, 95, 96, and or 123 of VHH-1 (SEQ ID NO: 27). In some embodiments, the antibody disclosed herein e.g., a single domain antibody e.g., a VHH antibody comprises a mutation e.g., an amino acid substitution at one or more amino acid residues 1, 5, 15, 24, 42, 49, 50, 52, 54, 55, 70, 80, 83, 87, 95, 96, and/or 123 relative to the wild type antibody e.g., a wild type single domain antibody e.g., a wild type VHH antibody of SEQ ID NO: 27, numbering according to IMGT scheme.

In some embodiments, the antibody disclosed herein e.g., a single domain antibody e.g., a VHH antibody has been optimized by introducing mutations, e.g., amino acid substitutions, for example at any one of positions of 1, 5, 15, 25, 42, 49, 50, 52, 54, 77, 78, 83, 87, 91, 92, 96, 103, 120 and/or 123 of VHH-2 (SEQ ID NO: 28). In some embodiments, the antibody disclosed herein e.g., a single domain antibody e.g., a VHH antibody comprises a mutation e.g., an amino acid substitution at one or more amino acid residues 1, 5, 15, 25, 42, 49, 50, 52, 54, 77, 78, 83, 87, 91, 92, 96, 103, 120 and/or 123 relative to the wild type antibody e.g., a wild type single domain antibody e.g., a wild type VHH antibody of SEQ ID NO: 28, numbering according to IMGT scheme.

These residues have been denoted by asterisks on FIG. 8A and FIG. 8B. Amino acid positions are numbered according to the international ImMunoGeneTics information system (IMGT), (Lefranc, M.-P. et al., 2009, Nucleic Acids Research, D1006-1012, http://www.imgt.org).

In some embodiments, the antibody e.g., a VHH antibody as described above, is an optimized humanized antibody e.g., single domain antibody e.g., a VHH antibody. In some embodiments, the antibody disclosed herein e.g., a single domain antibody e.g., a VHH antibody has been optimized by introducing mutations, e.g., amino acid substitutions, for example at any one of positions of 1, 5, 15, 24, 49, 50, 52, 70, 83, 95, 96 and/or 123 of VHH-1 (SEQ ID NO: 27). In some embodiments, the antibody disclosed herein e.g., a single domain antibody e.g., a VHH antibody comprises a mutation e.g., an amino acid substitution at one or more amino acid residues 1, 5, 15, 24, 49, 50, 52, 70, 83, 95, 96 and/or 123 relative to the wild type antibody e.g., a wild type single domain antibody e.g., a wild type VHH antibody of SEQ ID NO: 27, numbering according to IMGT scheme.

In some embodiments, the antibody disclosed herein e.g., a single domain antibody e.g., a VHH antibody has been optimized by introducing mutations, e.g., amino acid substitutions, for example at any one of positions of 1, 5, 15, 49, 50, 52, 77, 83, 84, 85, 86, 91, 92, 96, 103, 120, and/or 123 of VHH-2 (SEQ ID NO: 28). In some embodiments, the antibody disclosed herein e.g., a single domain antibody e.g., a VHH antibody comprises a mutation e.g., an amino acid substitution at one or more amino acid residues 1, 5, 15, 49, 50, 52, 77, 83, 84, 85, 86, 91, 92, 96, 103, 120, and/or 123 relative to the wild type antibody e.g., a wild type single domain antibody e.g., a wild type VHH antibody of SEQ ID NO: 28, numbering according to IMGT scheme.

In some embodiments, the antibody e.g., a VHH antibody as described above, is an optimized humanized antibody e.g., single domain antibody e.g., a VHH antibody. In some embodiments, the antibody disclosed herein e.g., a single domain antibody e.g., a VHH antibody has been optimized by introducing mutations, e.g., amino acid substitutions, for example at any one of positions of 1, 5, 15, 24, 49, 70, 83, 95, 96 and/or 123 of VHH-1 (SEQ ID NO: 27). In some embodiments, the antibody disclosed herein e.g., a single domain antibody e.g., a VHH antibody comprises a mutation e.g., an amino acid substitution at one or more amino acid residues 1, 5, 15, 24, 49, 70, 83, 95, 96 and/or 123 relative to the wild type antibody e.g., a wild type single domain antibody e.g., a wild type VHH antibody of SEQ ID NO: 27, numbering according to IMGT scheme.

In some embodiments, the antibody disclosed herein e.g., a single domain antibody e.g., a VHH antibody has been optimized by introducing mutations, e.g., amino acid substitutions, for example at any one of positions of 1, 5, 15, 49, 77, 83, 84, 85, 86, 91, 92, 96, 103, 120, and/or 123 of VHH-2 (SEQ ID NO: 28). In some embodiments, the antibody disclosed herein e.g., a single domain antibody e.g., a VHH antibody comprises a mutation e.g., an amino acid substitution at one or more amino acid residues 1, 5, 15, 49, 77, 83, 84, 85, 86, 91, 92, 96, 103, 120, and/or 123 relative to the wild type antibody e.g., a wild type single domain antibody e.g., a wild type VHH antibody of SEQ ID NO: 28, numbering according to IMGT scheme.

In some instances, the one or more mutations are to stabilize the antibody and/or to increase half-life. In some instances, the one or more mutations are to modulate Fc receptor interactions, to enhance Fc effector functions including, antibody-dependent cell-mediated cytotoxicity (ADCC), antibody-dependent cellular phagocytosis (ADCP) or complement-dependent cytotoxicity (CDC). In additional instances, the one or more mutations are to modulate glycosylation.

In some embodiments, the antibody comprises at least one modification in the Fc region that enhances cell killing. In some embodiments, the enhanced cell killing is enhanced antibody-dependent cellular cytotoxicity (ADCC) and/or complement-dependent cytotoxicity (CDC). In some embodiments, the at least one modification is a fucosylation. In some embodiments, the at least one modification is one or more heavy chain constant regions mutations at one or more positions selected from L234, L235, G236, S239, F243, H268, D270, R292, S298, Y300, V305, K326, A330, 1332, E333, K334, and P396. In some embodiments, the one or more heavy chain constant region mutations are one or more mutation selected from S239D, S239M, F243L, H268D, D270E, R292P, S298A, Y300L, V305I, K326D, A330L, A330M, 1332E, E333A, K334A, K334E, and P396L. In some embodiments, the one or more heavy chain constant region mutations are selected from: F243L/R292P/Y300L/V305I/P396L, S239D/1332E, S239D/1332E/A330L, S298A/E333A/K334A, L234Y/L235Q/G236W/S239M/H268D/D270E/S298A, and D270E/K326D/A330M/K334E.

In some instances, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC). In additional instances, the one or more mutations are to modulate glycosylation.

In some embodiments, the Fc region comprises a mutation at residue position L235, L236, D265, N297, K322, L328, or P329, or a combination of the mutations. In some instances, the Fc region comprises mutations at L235 and L236.

In some embodiments, an antibody described herein has an improved serum half-life compared to a reference anti-hCCR8 antibody. In some instances, the improved serum half-life is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than reference anti-hCCR8 antibody.

In some embodiments, the antibodies or antigen binding fragment thereof of the present disclosure are isolated. With regards to the isolated antibody; “isolated” is referred to when the antibody polypeptide is separated from at least some of the components of the cell (e.g., host cell) in which it was produced. Where a polypeptide is secreted by a host cell after expression, physically separating the supernatant containing the polypeptide from the host cell that produced it is considered to be “isolating” the polypeptide. The term “isolated” refers to a protein (e.g., an antibody) that is substantially free of other cellular material and/or chemicals. In one embodiment, an isolated antibody is substantially free of other proteins from the same species. In one embodiment, an isolated antibody is expressed by a cell from a different species and is substantially free of other proteins from the difference species. A protein may be rendered substantially free of naturally associated components (or components associated with the cellular expression system used to produce the antibody) by isolation, using protein purification techniques well known in the art. As used herein, the term “isolated antibody” also encompasses an antibody that is substantially free of other antibodies having different antigenic specificities (e.g., an isolated antibody that specifically binds to CCR8 (e.g., human CCR8) is substantially free of antibodies that specifically bind antigens other than CCR8). An isolated antibody that specifically binds to an epitope may, however, have cross-reactivity to other CCR8 proteins from different species. However, the antibody continues to display specific binding to human CCR8 in a specific binding assay as described herein. In addition, an isolated antibody is typically substantially free of other cellular material and/or chemicals. In some embodiments, a combination of “isolated” antibodies having different CCR8 specificities is combined in a well-defined composition. In some embodiments, the isolated antibody is a recombinant antibody.

It will also be understood by one of ordinary skill in the art that the antibodies suitable for use in the methods disclosed herein may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The antibodies suitable for use in the methods disclosed herein may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In certain aspects, a string of amino acids can be replaced with a structurally similar string that differs in order and/or composition of side-chain family members. Alternatively, in certain aspects, mutations may be introduced randomly along all or part of a coding sequence, such as by saturation mutagenesis, and the resultant mutants can be incorporated into binding polypeptides of the invention and screened for their ability to bind to the desired target.

As used herein, the term “epitope” or “antigenic determinant” refers to a site on an antigen (e.g., CCR8) to which an immunoglobulin or antibody specifically binds. The term “epitope mapping” refers to a process or method of identifying the binding site, or epitope, of an antibody, or antigen-binding fragment thereof, on its target protein antigen. Epitope mapping methods and techniques are provided herein. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from CCR8 are tested for reactivity with the given anti-CCR8 antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

Also encompassed by the present disclosure are antibodies that bind to an epitope on CCR8 which comprises all or a portion of an epitope recognized by the particular antibodies described herein (e.g., the same or an overlapping region or a region between or spanning the region).

Also encompassed by the present disclosure are antibodies that bind the same epitope and/or antibodies that compete for binding to human CCR8 with the antibodies described herein. Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as CCR8. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually, the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Other techniques include, for example, epitope mapping methods, such as, x-ray analyses of crystals of antigen:antibody complexes which provides atomic resolution of the epitope and mass spectrometry combined with hydrogen/deuterium (H/D) exchange which studies the conformation and dynamics of antigen:antibody interactions. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

### Single Domain Antibodies

In some embodiments, an antibody of the present disclosure is a single domain antibody. As used herein the terms “single domain antibody”, “heavy chain variable region domain of a heavy chain antibody”, “single variable domain”, “VHH”, “nanobody”, “sdAb” and “VHH domain” are used interchangeably, generally refers to a single variable region (e.g., VH) and specifically binds to a target antigen (e.g., CCR8). In other words, single variable domain of a single domain antibody does not need to recognize the target antigen (e.g., CCR8) by interacting with another variable region (e.g., VL). Examples of single domain antibodies include those derived from camelids (camels and llamas) and cartilaginous fish (e.g., nurse sharks) antibodies. Single domain antibodies are antibodies whose complementary determining regions are part of a single variable domain (e.g., CCR8 binding domain) polypeptide. Single domain antibodies thus contain single variable domain (e.g., a CCR8 binding domain) that contain a single CDR1, a single CDR2 and/or a single CDR3. In some embodiments, a single domain antibody comprises a single variable domain (e.g., a CCR8 binding domain) that comprises a single CDR1, a single CDR2, a single CDR3, or a combination thereof. Single domain antibodies may contain only the variable domain of an immunoglobulin chain having CDR1, CDR2, CDR3 or a combination thereof and in some embodiments further comprises one or more framework regions (e.g., one or more human framework regions). Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. According to one aspect of the invention, a single domain antibody as used herein is a naturally occurring single domain antibody known as heavy chain antibody devoid of light chains. Such single domain antibodies are disclosed in WO 9404678 for example. For clarity reasons, this variable domain devoid of a light chain or variable domain derived from a heavy chain antibody devoid of light chain is known herein as a VHH or nanobody to distinguish it from the conventional VH of four chain immunoglobulins. In some embodiments, the variable domain is derived from a derived from a heavy chain antibody naturally devoid of light chain. Such a VHH molecule can be derived from antibodies raised in Camelidae species, for example in camel, llama, dromedary, alpaca and guanaco. Accordingly, in some embodiments, an antibody of the present disclosure is a Camelidae VHH, or a llama VHH (or a llama single domain antibody). Other species besides Camelidae may produce heavy chain antibodies naturally devoid of light chain; such VHHs are within the scope of the invention. VHH molecules are about 10× smaller than IgG molecules. They are single polypeptides and very stable, resisting extreme pH and temperature conditions. Moreover, they are resistant to the action of proteases which is not the case for conventional antibodies. Furthermore, in vitro expression of VHHs produces high yield, properly folded functional VHHs. In addition, antibodies generated in Camelids will recognize epitopes other than those recognized by antibodies generated in vitro through the use of antibody libraries or via immunization of mammals other than Camelids (WO 9749805). In some embodiments, the single domain antibody is a VHH directed against a target, wherein the VHH belongs to a class having human-like sequences. The class is characterized in that the VHHs carry an amino acid from the group consisting of glycine, alanine, valine, leucine, isoleucine, proline, phenylalanine, tyrosine, tryptophan, methionine, serine, threonine, asparagine, or glutamine at position 45, such as, for example, L45 according to the Kabat numbering. As such, peptides belonging to this class show a high amino acid sequence homology to human VH framework regions and said peptides might be administered to a human directly without expectation of an unwanted immune response therefrom, and without the burden of further humanization.

In some embodiments, the single domain antibody is a Variable new antigen receptor (VNAR), variable domain of the immunoglobulin new antigen receptor (IgNAR) antibody derived from cartilaginous fish such as sharks. In some embodiments, a VNAR antibody comprise CRD1, and CDR2. In some embodiments, VNAR antibody further comprises two other hypervariable (HV) regions, referred to as the HV2 and HV4 regions. In some embodiments, a VNAR antibody further comprises one or more framework regions. The CDRs and HV regions are surrounded by framework (FW) regions in the following N-terminal to C-terminal order: FW1-CDR1-FW2-HV2-FW3 a-HV4-FW3b-CDR3-FW4. In some embodiments, the single domain antibody is a chimeric, synthetic, or humanized antibody. In some embodiments, the single domain antibody is multi-specific. In some embodiments, the single domain antibody is multivalent.

In some embodiments, an antibody of the present disclosure comprises two or more single domain antibodies. For example, an antibody disclosed herein can comprise at least two single domain antibodies. In some embodiments, the two single domain antibodies are directed to a same antigen (e.g., CCR8). Such multivalent polypeptide constructs have the advantage of unusually high functional affinity for the target, displaying much higher than expected inhibitory properties compared to their monovalent counterparts In some embodiments, an antibody of the present disclosure is bispecific antibody and comprises a first single domain antibody that comprises a CCR8 binding domain, and a second single domain antibody that comprises an antigen binding domain that specifically binds a target other than CCR8. For example, the second single domain antibody comprises an antigen binding domain that binds a target antigen (e.g., a cancer antigen).

In some embodiments, the two or more single domain antibodies are covalently linked. In some embodiments, two or more single domain antibodies may be identical in sequence or may be different in sequence, but are directed against the same target or antigen (e.g., CCR8). Depending on the number of single domain antibodies linked, a multivalent single domain antibody may be bivalent (2 single domain antibodies), trivalent (3 single domain antibodies), tetravalent (4 single domain antibodies) or have a higher valency molecules.

In some embodiments, two or more single domain antibodies may be different in sequence, and are directed against two different target or antigen. For example, an antibody of the disclosure can comprise a first single domain antibody comprising a CCR8 binding domain (i.e., directed against CCR8), and a second single domain antibody comprising an antigen binding domain that binds a target antigen (e.g., a cancer antigen). Depending on the number of single domain antibodies linked, a multispecific single domain antibody may be bispecific (2 single domain antibodies), trispecific (3 single domain antibodies), tetraspecific (4 single domain antibodies) or have a higher specificity molecules.

In some embodiments, the two or more single domain antibodies are linked using methods known in the art or any future method. They may be joined non-covalently (e.g., using streptavidin/biotin combination, antibody/tag combination) or covalently. They may be fused by chemical cross-linking by reacting amino acid residues with an organic derivatizing agent such as described by Blattler et al, Biochemistry 24, 1517-1524; EP294703. Alternatively, the single domain antibody may be fused genetically at the DNA level i.e., a polynucleotide construct formed which encodes the complete polypeptide construct comprising one or more single domain antibodies. A method for producing bivalent or multivalent single domain antibody is disclosed in PCT patent application WO 96/34103. One way of joining two or more single domain antibodies is via the genetic route by linking a single domain antibody coding sequences either directly or via a peptide linker. For example, the C-terminal end of the single domain antibody may be linked to the N-terminal end of the next single domain antibody.

This linking mode can be extended in order to link additional single domain antibodies for the construction and production of tri-, tetra-, etc. functional constructs. According to one aspect of the present invention, the single domain antibodies are linked to each other via a peptide linker sequence. Such linker sequence may be a naturally occurring sequence or a non-naturally occurring sequence. The linker sequence is expected to be non-immunogenic in the subject to which the multivalent anti-target polypeptide is administered. The linker sequence may provide sufficient flexibility to the multivalent anti-target polypeptide, at the same time being resistant to proteolytic degradation. A non-limiting example of a linker sequences is one that can be derived from the hinge region of VHHs described in WO 96/34103.

The polypeptide constructs disclosed herein may be made by the skilled artisan according to methods known in the art or any future method. For example, VHHs may be obtained using methods known in the art such as by immunizing a camel and obtaining hybridomas therefrom, or by cloning a library of single domain antibodies using molecular biology techniques known in the art and subsequent selection by using phage display or yeast display.

In some embodiments, an antibody disclosed herein further comprises a Fc region. Also provided herein are fusion proteins that include a single-domain antibody disclosed herein and a heterologous protein. In some embodiments, the heterologous protein is an Fc domain, such as a human Fc domain. Accordingly, in some embodiments, the single domain antibody is fused to a Fc domain (e.g., a human Fc domain).

In some embodiments, an antibody of the disclosure is a heavy chain only antibody. The term “heavy chain only antibody” refers to a single domain antibody that lacks a light chain and further comprises a CH2, a CH3 constant domains and/or at least a portion of a hinge region in absence of a CH1 domain.

### Species Selectivity and Species Cross-Reactivity

According to certain embodiments of the present disclosure, the antibodies can bind to human CCR8 but not to CCR8 from other species. Alternatively, the antibodies, in certain embodiments, bind to human CCR8 and to CCR8 from one or more non-human species. In some embodiments, the antibodies of the disclosure cross-reacts. For example, the antibodies or antigen binding fragment thereof can bind to human CCR8 and can bind or not bind, as the case may be, to one or more of mouse, rat, guinea pig, hamster, gerbil, pig, cat, dog, rabbit, goat, sheep, cow, horse, camel, cynomolgous, marmoset, rhesus or chimpanzee CCR8. As used herein, the term “cross-reacts” refers to the ability of an antibody of the disclosure to bind to CCR8 from a different species. For example, an antibody of the present disclosure that binds human CCR8 can also bind another species of CCR8. As used herein, cross-reactivity is measured by detecting a specific reactivity with purified antigen in binding assays (e.g., SPR, ELISA) or binding to, or otherwise functionally interacting with, cells physiologically expressing CCR8. Methods for determining cross-reactivity include standard binding assays as described herein, for example, by BIACORE™ surface plasmon resonance (SPR) analysis using a BIACORE™ 2000 SPR instrument (BIACORE AB, Uppsala, Sweden), or flow cytometric techniques.

### Nucleic Acids Encoding the Antibodies of the Disclosure

In one aspect provided herein is an isolated nucleic acid molecule encoding an antibody disclosed herein. Nucleotide sequences of the invention can be useful for a number of applications, including cloning, gene therapy, protein expression and purification, mutation introduction, DNA vaccination of a host in need thereof, antibody generation for, e.g., passive immunization, PCR, primer and probe generation, and the like.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides.

As used herein, an “isolated” nucleic acid molecule or “isolated” nucleic acid sequence is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined, is considered isolated. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells. “Isolated nucleic acid”, as used herein, is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man. In some embodiments, the isolated nucleic acid molecule of the disclosure encompasses nucleic acids encoding antibodies disclosed herein or antibody portions (e.g., VH, CDR1, CDR2, CDR3) that bind to CCR8, in which the nucleic acids encoding the antibody or antibody portion are free of other nucleotide sequences encoding antibodies or antibody portions that bind antigens other than CCR8, which other sequences may naturally flank the nucleic acid in human genomic DNA.

An isolated nucleic acid molecule encoding the antibody, portion or polypeptide of the present disclosure can be recombined with vector DNA (e.g., expression vector) in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., Molecular Cloning, Lab. Manual (Cold Spring Harbor Lab. Press, NY, 1982 and 1989), and Ausubel, 1987, 1993, and can be used to construct nucleic acid sequences which encode an antibody molecule. Accordingly, the disclosure provides for a vector or expression vector comprising the isolated nucleic acids set forth herein. Once the isolated nucleic acid molecule is placed into an expression vector, they can then be transfected into host cells such as E. coli cells, simian COS cells, human embryonic kidney 293 cells (e.g., 293E cells), Chinese hamster ovary (CHO) cells, or myeloma cells that do not otherwise produce immunoglobulin protein, to obtain an antibody disclosed herein in the recombinant host cells. Recombinant production of antibodies is well known in the art. Many vectors are available. The vector components generally include, but are not limited to, one or more of the following: a signal sequence, an origin of replication, one or more selective marker genes, an enhancer element, a promoter, and a transcription termination sequence.

Amino acid sequence variants of the desired antibody may be prepared by introducing appropriate nucleotide changes into the encoding DNA, or by peptide synthesis. Such variants include, for example, deletions from, and/or insertions into and/or substitutions of, residues within the amino acid sequences of the antibodies. Any combination of deletion, insertion, and substitution is made to arrive at the final construct, provided that the final construct possesses the desired characteristics (e.g., binding to CCR8). The amino acid changes also may alter post-translational processes of the monoclonal, human, humanized, or variant antibody, such as changing the number or position of glycosylation sites.

Nucleic acid molecules encoding amino acid sequence variants of the antibody are prepared by various methods. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

In some embodiments, the antibody or antigen binding fragment thereof disclosed herein is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003). For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system. Antibody polypeptides can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis. In some embodiments, the antibodies or antigen binding fragment of the present disclosure is synthetic. The polypeptides of the present disclosure can be purified by isolation/purification methods for proteins generally known in the field of protein chemistry.

### Antigen—CCR8

Provided herein are antibodies that specifically bind a CCR8 or a portion thereof. As used herein, the term “CCR8” or “C-C chemokine receptor type 8” or “CCR8 polypeptide” refers to a G-protein coupled receptor polypeptide. The term “CCR8” of the disclosure refers to a human CCR8 polypeptide. The term “CCR8” encompasses any native or variant (whether native or synthetic) CCR8. The term “native” CCR8 encompasses naturally occurring truncated or secreted forms (e.g., an extracellular domain sequence; e.g., as set forth in SEQ ID NO: 38), naturally occurring variant forms (e.g., alternatively spliced forms) and naturally-occurring allelic variants. The term “CCR8” as used herein refers to the 355 amino acid polypeptide set forth in SEQ ID NO: 37 together with the naturally occurring allelic and processed forms thereof. The term “CCR8” includes a CCR8 variant. As used herein, the term “CCR8” refers to a full length CCR8 polypeptide or to a portion or derivative thereof. The CCR8 polypeptide can be a full length human CCR8 and/or functional fragments thereof, a species homologue and/or functional fragments thereof, an ortholog of human CCR8 and/or functional fragments thereof. The CCR8 polypeptide can be a mammalian CCR8 polypeptide. The CCR8 can also be a functional isoform of the full length CCR8 or a portion thereof. In some embodiments, the CCR8 includes or is derived from a human CCR8 amino acid sequence provided in SEQ ID NO: 37.

The term “CCR8” also includes truncated forms human CCR8. In some embodiments, CCR8 is a truncated form of the 355 amino acid human CCR8 (e.g., SEQ ID NO: 37). The term “CCR8 variant” as used herein refers to a CCR8 polypeptide which includes one or more amino acid mutations in the native CCR8 sequence (e.g., SEQ ID NO: 37). Optionally, the one or more amino acid mutations include amino acid substitution(s). CCR8 is known to have at least four ligands: CCL1, CCL8, CCL16, and CCL18. CCL1 is thought to potentiate human Treg cells by inducing CCR8, FOXp3, CD39, Granzyme B, and IL-10 Expression, in a STAT3-dependent manner. See, e.g., Barsheshet et al., PNAS 114(23):6086-91 (Jun. 6, 2017). CCR8 is expressed primarily on Treg cells and to a lesser extent on small fractions of TH2 cells, monocytic cells, NK cells, and CD8+ cells. CCR8 is a transmembrane receptor having seven transmembrane domains, an extracellular N-terminal domain (SEQ ID NO: 38), and an intracellular C-terminal domain, which interacts with G-protein. In some embodiments, the CCR8 is expressed in a cell (e.g., on the surface of a cell). In some embodiments, the cell is a regulatory T cell, a tumor infiltrating T cell, a TH2 cell, an NK cell, a monocytic cell, a CD8+ cell, or a cancer cell. In some embodiments, the CCR8 is expressed on the surface of a cell. In some embodiments, the CCR8 is soluble. In some embodiments the CCR8 is recombinant. In some embodiments, an antibody disclosed herein specifically binds a transmembrane domain of a CCR8 (e.g., human CCR8).

### Antibody Conjugate

In some embodiments, an antibody described above is further conjugated to a payload. In some instances, the payload comprises a small molecule. In other instances, the payload comprises a protein or a peptide. In additional instances, the payload comprises a polynucleic acid molecule.

In some instances, a ratio of the payload to the anti-hCCR8 antibody (drug-to-antibody ratio or DAR ratio) is about 1:1, 2:1, 3:1, 4:1, 5:1, 6:1, 7:1, 8:1, 9:1, 10:1, 11:1, 12:1, 13:1, 14:1, 15:1, or 16:1.

In some cases, an anti-hCCR8 antibody conjugate comprises

$$A - {( {X}^{1} - B )}_{n} \qquad Formula ( I )$$

wherein,
A comprises the anti-hCCR8 antibody;
B comprises the payload;
X¹ consists of a bond or linker; and
n is an averaged value selected from 1-12.

In some instances, the DAR ratio of B to A (the anti-hCCR8 antibody) is about 1, 2, 3, 4, 5, 6, 7, 8, 9, 10, 11, 12, 13, 14, 15, or 16. In some cases, the DAR ratio of B to A is about 1. In some cases, the DAR ratio of B to A is about 2. In some cases, the DAR ratio of B to A is about 3. In some cases, the DAR ratio of B to A is about 4. In some cases, the DAR ratio of B to A is about 6. In some cases, the DAR ratio of B to A is about 8. In some cases, the DAR ratio of B to A is about 10. In some cases, the DAR ratio of B to A is about 12. In some cases, the DAR ratio of B to A is about 16.

In some instances, the DAR ratio of the polynucleic acid molecule (B) to anti-hCCR8 antibody A is about 1, 2, 3, 4, 5, 6, 7, 8, 9, 10, 11, 12, 13, 14, 15, or 16. In some instances, the DAR ratio of the polynucleic acid molecule (B) to anti-hCCR8 antibody A is about 1. In some instances, the DAR ratio of the polynucleic acid molecule (B) to anti-hCCR8 antibody A is about 2. In some instances, the DAR ratio of the polynucleic acid molecule (B) to anti-hCCR8 antibody A is about 3. In some instances, the DAR ratio of the polynucleic acid molecule (B) to anti-hCCR8 antibody A is about 4. In some instances, the DAR ratio of the polynucleic acid molecule (B) to anti-hCCR8 antibody A is about 5. In some instances, the DAR ratio of the polynucleic acid molecule (B) to anti-hCCR8 antibody A is about 6. In some instances, the DAR ratio of the polynucleic acid molecule (B) to anti-hCCR8 antibody A is about 7. In some instances, the DAR ratio of the polynucleic acid molecule (B) to anti-hCCR8 antibody A is about 8. In some instances, the DAR ratio of the polynucleic acid molecule (B) to anti-hCCR8 antibody A is about 9. In some instances, the DAR ratio of the polynucleic acid molecule (B) to anti-hCCR8 antibody A is about 10. In some instances, the DAR ratio of the polynucleic acid molecule (B) to anti-hCCR8 antibody A is about 11. In some instances, the DAR ratio of the polynucleic acid molecule (B) to anti-hCCR8 antibody A is about 12. In some instances, the DAR ratio of the polynucleic acid molecule (B) to anti-hCCR8 antibody A is about 13. In some instances, the DAR ratio of the polynucleic acid molecule (B) to anti-hCCR8 antibody A is about 14. In some instances, the DAR ratio of the polynucleic acid molecule (B) to anti-hCCR8 antibody A is about 15. In some instances, the DAR ratio of the polynucleic acid molecule (B) to anti-hCCR8 antibody A is about 16.

In some embodiments, B comprises a small molecule, a peptide, or a protein.

In some embodiments, B comprises a polynucleic acid molecule. In some cases, the polynucleic acid molecule comprises a passenger strand and a guide strand. In some cases, the passenger strand is conjugated to A-X¹. In some cases, A-X¹ is conjugated to the 5′ end of the passenger strand. In some cases, A-X¹ is conjugated to the 3′ end of the passenger strand.

In some cases, an anti-hCCR8 antibody conjugate comprises

$$A - {X}^{1} - {( B - {X}^{2} - C )}_{n} \qquad Formula ( II )$$

wherein,
A comprises the anti-hCCR8 antibody;
B comprises the payload;
C consists of a polymer;
X¹ consists a bond or first linker;
X² consists of a bond or second linker; and
n is an averaged value selected from 1-12.

In some embodiments, C is a polyethylene glycol.

In some embodiments, B is a polynucleic acid molecule. In some cases, the polynucleic acid molecule comprises a passenger strand and a guide strand. In some cases, the passenger strand is conjugated to A-X¹ and X²—C. In some cases, A-X¹ is conjugated to the 5′ end of the passenger strand and X²—C is conjugated to the 3′ end of the passenger strand. In some cases, X²—C is conjugated to the 5′ end of the passenger strand and A-X¹ is conjugated to the 3′ end of the passenger strand.

In some embodiments, X¹ and X² are each independently a non-polymeric linker.

In some embodiments, the conjugate of Formula (II) A-X¹—(B—X²—C)ₙ further comprises D, an endosomolytic moiety.

### Conjugation Chemistry

In some embodiments, B is conjugated to A by a chemical ligation process. In some instances, B is conjugated to A by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some embodiments, the polynucleic acid molecule is conjugated to the binding moiety either site-specifically or non-specifically via native ligation chemistry.

In some instances, B is conjugated to A by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012)).

In some instances, B is conjugated to A by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivative conjugating moiety to form an oxime bond. (see Axup et al., “Synthesis of site-specific antibody-drug conjugates using unnatural amino acids,” PNAS 109(40): 16101-16106 (2012)).

In some instances, B is conjugated to A by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some embodiments, the enzyme-catalyzed process comprises microbial transglutaminase (mTG). In some cases, B is conjugated to A utilizing a microbial transglutaminase catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some instances, B is conjugated to A by a method as described in PCT Publication No. WO2014/140317, which utilizes a sequence-specific transpeptidase.

In some instances, B is conjugated to A by a method as described in U.S. Patent Publication Nos. 2015/0105539 and 2015/0105540.

Conjugates of the antibodies described herein can be also made using any of a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., 238 Science 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026.

Techniques for conjugating such payload to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987).

In some embodiments, the payload can be attached at the hinge region of a reduced antibody component via disulfide bond formation. For example, the tetanus toxoid peptides can be conjugated with a single cysteine residue that is used to attach the peptide to an antibody component.

In some embodiments, the payload can be conjugated to a Fc region of the antibody (e.g., carbohydrate moieties in the Fc region of an antibody can be used to conjugate a therapeutic agent. In some embodiments, the payload is conjugated to a variable region of the antibody (e.g., it is possible to introduce a carbohydrate moiety into the variable region of an antibody or antibody fragment. See, for example, Leung et al., J. Immunol. 154:5919 (1995); Hansen et al., U.S. Pat. No. 5,443,953. The engineered carbohydrate moiety is then used to attach a payload. In addition, those of skill in the art will recognize numerous possible variations of the conjugation methods. For example, the carbohydrate moiety can be used to attach polyethylene glycol in order to extend the half-life of an intact antibody, or antigen-binding fragment thereof, in blood, lymph, or other extracellular fluids. Moreover, it is possible to construct a “divalent conjugate” by attaching therapeutic agents to a carbohydrate moiety and to a free sulfhydryl group. Such a free sulfhydryl group may be located in the hinge region of the antibody component.

### Payloads

In some embodiments, the payload is a polynucleic acid molecule. In some instances, the polynucleic acid molecule hybridizes to a target region of an oncogene. Exemplary oncogenes include, but are not limited to, Abl, AKT-2, ALK, AMLI (or R UNXI), AR, AXL, BCL-2, 3, 6, BRAF, c-MYC, EGFR, ErbB-2 (Her2, Neu), Fins, FOS, GLI1, HPRT1, IL-3, INTS2, JUN, KIT, KS3, K-sam, LBC (AKAP13), LCK, LMO1, LMO2, LYL1, MAS1, MDM2, MET, MLL (KMT2A), MOS, MYB, MYHl1/CBFB, NOTCH1 (TAN1), NTRK1 (TRK), OST (SLC51B), PAXS, PIM1, PRAD-1, RAF, RAR/PML, HRAS, KRAS, NRAS, REL/NRG, RET, ROS, SKI, SRC, TIAM1, or TSC2. In some cases, the polynucleic acid molecule hybridizes to a target region of KRAS, EGFR, AR, HPRT1, CNNTB1 (β-catenin), or β-catenin associated genes.

### Small Molecules, Proteins, and Peptides

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some instances, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapnstone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises a protein or peptide toxin or fragment thereof. Exemplary enzymatically active toxins and fragments thereof include, but are not limited to, diphtheria toxin A fragment, nonbinding active fragments of diphtheria toxin, exotoxin A (from Pseudomonas aeruginosa), ricin A chain, abrin A chain, modeccin A chain, a-sacrin, certain A leurites fordii proteins, certain Dianthin proteins, Phytolacca americana proteins (PAP, PAPII and PAP-S), Momordica charantia inhibitor, curcin, crotin, Saponaria officinalis inhibitor, gelonin, mitogillin, restrictocin, phenomycin, enomycin, and tricothecenes.

In some embodiments, the payload is an immune modulator. Exemplary immune modulators include, but are not limited to, gancyclovier, etanercept, tacrolimus, sirolimus, voclosporin, cyclosporine, rapamycin, cyclophosphamide, azathioprine, mycophenolgate mofetil, methotrextrate, glucocorticoid and its analogs, xanthines, stem cell growth factors, lymphotoxins, hematopoietic factors, tumor necrosis factor (TNF) (e.g., TNFα), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S1 factor,” erythropoietin and thrombopoietin, or a combination thereof.

In some instances, the payload comprises a cytokine. In some embodiments, the cytokine comprises IL-2, IL-2, IL-3, IL-6, IL-10, IL-12, IL-18, IL-21, interferon (e.g., IFNα, IFNβ), or TNFα.

The present disclosure also contemplated conjugation of more than one payloads to an antibody of the disclosure. For example, in some embodiments, an antibody of the disclosure can be conjugated to at least 1, 2, 3, 4, 5, 6, 7, 8, 9, or 10 payloads. In one embodiment, the antibody is used as a radiosensitizer. In such embodiments, the antibody is conjugated to a radiosensitizing agent. Accordingly in some embodiments, the payload comprises a radiosensitizing agent. The term “radiosensitizer,” as used herein, is defined as a molecule, preferably a low molecular weight molecule, administered to animals in therapeutically effective amounts to increase the sensitivity of the cells to be radiosensitized to electromagnetic radiation and/or to promote the treatment of diseases that are treatable with electromagnetic radiation. Diseases that are treatable with electromagnetic radiation include neoplastic diseases, benign and malignant tumors, and cancerous cells. In another embodiment, the antibody may be conjugated to a receptor (such streptavidin) for utilization in tumor pre-targeting e.g., the antibody may be used for tumor site or a tumor microenvironment (e.g., a tumor site or a tumor microenvironment that comprises a cell expressing a CCR8 polypeptide), wherein the antibody-receptor conjugate is administered to the patient, followed by removal of unbound conjugate from the circulation using a clearing agent and then administration of a ligand (e.g., avidin) which is conjugated to a cytotoxic agent (e.g., a radionuclide). In some embodiments, payload comprises biotin, and the biotin conjugated antibody or antibody fragment thereof can be further conjugated or linked to a streptavidin-bound or -coated agent, such as a streptavidin-coated microbubble, for use in, for example, molecular imaging of angiogenesis.

The present disclosure further provides the above-described antibodies in detectably labeled form. Antibodies can be conjugated with a detectable label. In some embodiments, the payload is a label, e.g., a radioisotope, an affinity label (such as biotin, avidin, etc.), an enzymatic label (such as horseradish peroxidase, alkaline phosphatase, etc.), fluorescent or luminescent or bioluminescent labels (such as FITC or rhodamine, etc.), paramagnetic atoms, and the like. In some embodiments, the payload comprises an enzyme, fluorescent marker, chemiluminescent marker, bioluminescent material, or radioactive material. Procedures for accomplishing such labeling are well known in the art; for example, see (Sternberger, L. A. et al., J. Histochem. Cytochem. 18:315 (1970); Bayer, E. A. et al., Meth. Enzym. 62:308 (1979); Engval, E. et al., Immunol. 109:129 (1972); Goding, J. W. J. Immunol. Meth. 13:215 (1976)).

The detectable label may itself be detectable by itself (e.g., radioisotope labels or fluorescent labels) or, in the case of an enzymatic label, may catalyze chemical alteration of a substrate compound or composition which is detectable. Alternatively, the label may not be detectable on its own but may be an element that is bound by another agent that is detectable (e.g., an epitope tag or one of a binding partner pair such as biotin-avidin, etc.). Thus, the antibody may comprise a label or tag that facilitates its isolation, and methods of the invention to identify antibodies include a step of isolating the antigen/antibody through interaction with the label or tag.

In some embodiments, the payload comprises a chemotherapeutic agent, toxin (e.g., an enzymatically active toxin of bacterial, fungal, plant or animal origin, or fragments thereof), or a radioactive isotope (i.e., a radioconjugate).

In some embodiments, the payload comprises a therapeutic agent such as a chemotherapeutic cytotoxin, such as a cytostatic or cytocidal agent (e.g., paclitaxol, cytochalasin B or diphtheria toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracindione, mitoxantrone, mithramycin, actinomycin D, 1-dehydrotestosterone, glucocorticoids, procaine, tetracaine, lidocaine, propranolol, and puromycin and analogs or homologs thereof.), antimetabolites (e.g., methotrexate, 6-mercaptopurine, 6-thioguanine, cytarabine, fludarabin, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents, a thrombotic or anti-angiogenic agent or a radioactive label. Examples of suitable cytotoxic agents and chemotherapeutic agents for forming immunoconjugates are known in the art, see for example, WO 05/103081).

In some embodiments, the payload comprises a toxin (e.g., an enzymatically active toxin of bacterial, fungal, plant or animal origin, or fragments thereof), a small molecule, an siRNA, a nanoparticle, a targeting agent (e.g., a microbubble), or a radioactive isotope (i.e., a radioconjugate). The antibody conjugates can be used, for example, in diagnostic, theranostic, or targeting methods. A variety of radioisotopes are available for use as payloads with the antibodies of the disclosure. Examples include, but are not limited to, 212 Bi, 131 I, 131 In, 90Y and 186Re.

### Linkers

In some embodiments, a linker described herein is a cleavable linker or a non-cleavable linker. In some instances, the linker is a cleavable linker. In other instances, the linker is a non-cleavable linker.

In some embodiments, the linker is a non-polymeric linker. A non-polymeric linker refers to a linker that does not contain a repeating unit of monomers generated by a polymerization process. Exemplary non-polymeric linkers include, but are not limited to, C₁-C₆ alkyl group (e.g., a C₅, C₄, C₃, C₂, or C₁ alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C₅, C₄, C₃, C₂, or C₁ alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups.

In some embodiments, the non-polymeric linker does not encompass a polymer that is described above. In some instances, the non-polymeric linker does not encompass a polymer encompassed by the polymer moiety C. In some embodiments, the non-polymeric linker does not encompass a polyalkylene oxide (e.g., PEG). In some embodiments, the non-polymeric linker does not encompass a PEG.

In some embodiments, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (pNPDP), ρ-nitrophenyl-2-diazo-3,3,3-trifluoropropionate (PNP-DTP), sulfhydryl-reactive and photoreactive cross-linkers such as 1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some embodiments, the linker comprises a reactive functional group. In some cases, the reactive functional group comprises a nucleophilic group that is reactive to an electrophilic group present on a binding moiety. Exemplary electrophilic groups include carbonyl groups-such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some embodiments, the linker comprises a peptide moiety. In some instances, the peptide moiety comprises at least 2, 3, 4, 5, or 6 more amino acid residues. In some instances, the peptide moiety comprises at most 2, 3, 4, 5, 6, 7, or 8 amino acid residues. In some instances, the peptide moiety comprises about 2, about 3, about 4, about 5, or about 6 amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly (SEQ ID NO: 247), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 248), or Gly-Phe-Leu-Gly (SEQ ID NO: 249). In some instances, the linker comprises a peptide moiety such as: Val-Cit (valine-citrulline), Gly-Gly-Phe-Gly (SEQ ID NO: 247), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 248), or Gly-Phe-Leu-Gly (SEQ ID NO: 249). In some cases, the linker comprises Val-Cit. In some cases, the linker is Val-Cit.

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker is used to increase the molar ratio of polynucleotide B to the binding moiety A. In some instances, the dendritic type linker comprises PAMAM dendrimers.

In some embodiments, the linker is a traceless linker or a linker in which after cleavage does not leave behind a linker moiety (e.g., an atom or a linker group) to a binding moiety A, a polynucleotide B, a polymer C, or an endosomolytic moiety D. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042.

In some embodiments, X¹ and X² are each independently a bond or a non-polymeric linker. In some instances, X¹ and X² are each independently a bond. In some cases, X¹ and X² are each independently a non-polymeric linker.

In some instances, X¹ is a bond or a non-polymeric linker. In some instances, X¹ is a bond. In some instances, X¹ is a non-polymeric linker. In some instances, the linker is a C₁-C₆ alkyl group. In some cases, X¹ is a C₁-C₆ alkyl group, such as for example, a C₅, C₄, C₃, C₂, or C₁ alkyl group. In some cases, the C₁-C₆ alkyl group is an unsubstituted C₁-C₆ alkyl group. As used in the context of a linker, and in particular in the context of X¹, alkyl means a saturated straight or branched hydrocarbon radical containing up to six carbon atoms. In some instances, X¹ includes a homobifunctional linker or a heterobifunctional linker described supra. In some cases, X¹ includes a heterobifunctional linker. In some cases, X¹ includes sMCC. In other instances, X¹ includes a heterobifunctional linker optionally conjugated to a C₁-C₆ alkyl group. In other instances, X¹ includes sMCC optionally conjugated to a C₁-C₆ alkyl group. In additional instances, X¹ does not include a homobifunctional linker or a heterobifunctional linker described supra.

In some instances, X² is a bond or a linker. In some instances, X² is a bond. In other cases, X² is a linker. In additional cases, X² is a non-polymeric linker. In some embodiments, X² is a C₁-C₆ alkyl group. In some instances, X² is a homobifunctional linker or a heterobifunctional linker described supra. In some instances, X² is a homobifunctional linker described supra. In some instances, X² is a heterobifunctional linker described supra. In some instances, X² comprises a maleimide group, such as maleimidocaproyl (mc) or a self-stabilizing maleimide group described above. In some instances, X² comprises a peptide moiety, such as Val-Cit. In some instances, X² comprises a benzoic acid group, such as PABA. In additional instances, X² comprises a combination of a maleimide group, a peptide moiety, and/or a benzoic acid group. In additional instances, X² comprises a me group. In additional instances, X² comprises a mc-val-cit group. In additional instances, X² comprises a val-cit-PABA group. In additional instances, X² comprises a mc-val-cit-PABA group.

### Pharmaceutical Compositions and Medicaments

In one aspect, provided herein is a pharmaceutical composition, comprising an antibody of the disclosure. The antibodies, or the compositions, described herein can be used for treatment of a disease, prevention of a disease, screening for a disease, detecting a presence or a severity of a disease, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. In some embodiments, the disease is cancer.

For the clinical use of the methods described herein, administration of an antibody of the present disclosure, can include formulation into pharmaceutical compositions or pharmaceutical formulations, or medicaments for administration, e.g., subcutaneous, intravenous, intradermal, intraperitoneal, oral, intramuscular, intracranial or other routes of administration. In some embodiments, an antibody described herein can be administered along with any pharmaceutically acceptable carrier, excipient, or diluent, which results in an effective treatment in the subject. Thus, in one aspect, the present disclosure provides pharmaceutical compositions comprising one or more antibodies described herein, in combination with one or more pharmaceutically acceptable carrier, excipient, or diluent.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an antibody or antigen binding fragment thereof of the present disclosure. Examples include, but are not limited to, any of a number of standard pharmaceutical carriers such as sterile phosphate buffered saline solutions, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like. Each carrier must be “acceptable” in the sense of being compatible with the other ingredients of the formulation and not injurious to the patient. The terms “excipient”, “carrier”, “pharmaceutically acceptable carrier”, or the like are used interchangeably herein. The compositions of the present disclosure may further comprise one or more pharmaceutically acceptable carriers, excipients, and other agents that are incorporated into formulations to provide improved transfer, delivery, tolerance, and the like (herein collectively referred to as “pharmaceutically acceptable carriers or diluents”). A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA, 1998, J Pharm Sci Technol 52:238-311.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Optionally, the formulations comprising the compositions described herein contain a pharmaceutically acceptable salt, typically, e.g., sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. Optionally, the formulations of the invention can include a pharmaceutically acceptable surfactant at a concentration of 0.005 to 0.02%.

The compositions described herein can be specially formulated for administration of the antibody or antigen binding fragment thereof to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (7) nasally. Additionally, an antibody or antigen binding fragment thereof, or compositions of the present disclosure can be implanted into a patient or injected using a drug delivery system. See, e.g., Urquhart et al., 24 Ann. Rev. Pharmacol. Toxicol. 199 (1984); Controlled Release of Pesticides & Pharmaceuticals (Lewis, ed., Plenum Press, New York, 1981); U.S. Pat. Nos. 3,773,919, 3,270,960.

The compositions disclosed herein, comprising an antibody described herein, can also contain more than one active compound as necessary for the particular indication being treated, preferably those with complementary activities that do not adversely affect each other. For example, the composition can further comprise a therapeutic agent, an anti-cancer agent, cytotoxic agent, cytokine, growth inhibitory agent and/or an angiogenesis inhibitor such as a VEGFR antagonist. Such molecules are suitably present in combination in amounts that are effective for the purpose intended. The active ingredients of the compositions comprising an antibody described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microparticle, microemulsions, nano-particles and nanocapsules) or in macroemulsions. Such techniques are disclosed in Remington's Pharmaceutical Sciences (16th ed., Osol, ed., 1980). The pharmaceutical composition can be also delivered in a vesicle, in particular a liposome (see Langer 1990 Science 249:1527-1533; Treat et al. (1989) in Liposomes in the Therapy of Infectious Disease and Cancer, Lopez-Berestein and Fidler (eds.), Liss, New York, pp. 353-365; Lopez-Berestein, ibid., pp. 317-327; see generally ibid.). Liposomes include emulsions, foams, micelles, insoluble monolayers, phospholipid dispersions, lamellar layers and the like, and can serve as vehicles to target the M-CSF antibodies to a particular tissue as well as to increase the half-life of the composition. A variety of methods are available for preparing liposomes, as described in, e.g., U.S. Pat. Nos. 4,837,028 and 5,019,369, which patents are incorporated herein by reference.

In some embodiments, an antibody of the present invention can be conjugated to the liposomes as described in Martin et al., J. Biol. Chem. 257: 286-288 (1982) via a disulfide interchange reaction.

In some embodiments, sustained-release preparations can be used. Suitable examples of sustained-release preparations include semipermeable matrices of solid hydrophobic polymers containing an antibody or antigen binding fragment of the present disclosure, in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thiodisulfide interchange, stabilization can be achieved by modifying sulfhydryl residues, lyophilizing from acidic solutions, controlling moisture content, using appropriate additives, and developing specific polymer matrix compositions. In certain situations, the pharmaceutical composition can be delivered in a controlled release system. In one embodiment, a pump may be used (see Langer, supra; Sefton 1987 CRC Crit. Ref. Biomed. Eng. 14:201). In another embodiment, polymeric materials can be used. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138).

A pharmaceutical composition of the present disclosure can be delivered, e.g., subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN70130™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition include, but certainly are not limited to the SOLOSTAR™ pen (Sanofi-Aventis), the FLEXPEN™ (Novo Nordisk), and the KWIKPEN™ (Eli Lilly).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Compositions of the present disclosure can be in the form of, for example, granules, powders, tablets, capsules, syrup, suppositories, injections, emulsions, elixirs, suspensions or solutions. The amount of the aforesaid antibody contained can be about 5 to about 500 mg per dosage form in a unit dose; especially in the form of injection, it is preferred that the aforesaid antibody is contained in about 5 to about 100 mg and in about 10 to about 250 mg for the other dosage forms.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. In some embodiments, pharmaceutical formulations and medicaments may be prepared as liquid suspensions or aqueous solutions, for example, using a sterile liquid, such as, but not limited to, an oil, water, an alcohol, and combinations of these. In some embodiments, pharmaceutical compositions can be prepared in a lyophilized form. The lyophilized preparations can comprise a cryoprotectant known in the art. The term “cryoprotectants” as used herein generally includes agents, which provide stability to the protein from freezing-induced stresses. Examples of cryoprotectants include polyols such as, for example, mannitol, and include saccharides such as, for example, sucrose, as well as including surfactants such as, for example, polysorbate, poloxamer or polyethylene glycol, and the like. Cryoprotectants also contribute to the tonicity of the formulations. Pharmaceutically suitable surfactants, suspending agents, emulsifying agents, may be added for oral or par-enteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, iso-propyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For nasal administration, the pharmaceutical formulations and medicaments may be a spray or aerosol containing an appropriate solvent(s) and optionally other compounds such as, but not limited to, stabilizers, antimicrobial agents, antioxidants, pH modifiers, surfactants, bio-availability modifiers and combinations of these. A propellant for an aerosol formulation may include compressed air, nitrogen, carbon dioxide, or a hydrocarbon based low boiling solvent.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

The concentration of an antibody or an antigen binding fragment thereof in these compositions can vary widely, i.e., from less than about 10%, usually at least about 25% to as much as 75% or 90% by weight and will be selected primarily by fluid volumes, viscosities, etc., in accordance with the particular mode of administration selected. Actual methods for preparing orally, topically and parenterally administrable compositions will be known or apparent to those skilled in the art and are described in detail in, for example, Remington's Pharmaceutical Science, 19th ed., Mack Publishing Co., Easton, Pa. (1995), which is incorporated herein by reference.

In another embodiment of the invention, an article of manufacture containing materials useful for the treatment of the diseases, disorders or conditions described above is provided, including for treatment of cancer. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the antibody of the invention. The label on or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user stand-point, including other buffers, diluents, filters, needles, syringes, and package inserts with instructions for use. Pharmaceutical compositions and medicaments described herein are useful in treating a cancerous disease.

### Production of Antibodies or Binding Fragments Thereof

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification or analysis of an antibody or antibody conjugates is used, for example, by chromatography (e.g., ion exchange, affinity, particularly by affinity for the specific antigen after Protein A, and sizing column chromatography), centrifugation, differential solubility, or by any other standard technique for the purification of proteins. Exemplary chromatography methods included, but are not limited to, strong anion exchange chromatography, hydrophobic interaction chromatography, size exclusion chromatography, and fast protein liquid chromatography.

### Methods of the Disclosure

Certain aspects of the present disclosure are directed to methods of making and/or using the antibodies disclosed herein.

Certain aspects of the present disclosure are directed to methods of reducing, decreasing, depleting, or killing cells expressing CCR8 (e.g., Treg cells, e.g., tumor infiltrating Tregs), comprising administering to the subject an antibody disclosed herein or a composition disclosed herein, or contacting a population of cells expressing CCR8 (e.g., Treg cells, e.g., tumor infiltrating Tregs) with an antibody disclosed herein or a composition disclosed herein. In one aspect provided herein is a method for enhancing an immune response in a subject comprising administering to the subject an antibody disclosed herein or a composition disclosed herein. The terms “inducing an immune response” and “enhancing an immune response” are used interchangeably and refer to the stimulation of an immune response (i.e., either passive or adaptive) to a particular antigen (e.g., a cancer antigen). The terms “induce” as used with respect to inducing CDC or ADCC refer to the stimulation of particular direct cell killing mechanisms. In some embodiments, the enhancing is relative to a reference level. In some embodiments, the enhancing is at least about 2, 5, 10, 15, 20, 25, 30, 35, 40, 45, 50, 55, 60, 65, 70, 75, 80, 85, 90, 91, 92, 93, 94, 95, 96, 97, 98, 99, 100% or more relative to a reference level. In some embodiments, the enhancing is at least 2, 5, 10, 15, 20, 25, 30, 35, 40, 45, 50, 55, 60, 65, 70, 75, 80, 85, 90, 91, 92, 93, 94, 95, 96, 97, 98, 99, 100% or more relative to a reference level. In some embodiments, the immune response is an anti-cancer immune response. As used herein, the term “anti-cancer immune response” refers to the immune response induced by the presence of tumors, cancer cells, or cancer antigens. In some embodiments, the response includes the proliferation of cancer antigen specific lymphocytes. In some embodiments, the response includes expression and upregulation of antibodies and T-cell receptors and the formation and release of lymphokines, chemokines, and cytokines. Both innate and acquired immune systems interact to initiate antigenic responses against the tumors, cancer cells, or cancer antigens. In some embodiments, the immune response (e.g., an anti-cancer immune response) is a T cell response. As used herein, the term “T cell response” refers to any response mediated by T cells, including, but not limited to, effector T cells (e.g., CD8+ cells) and helper T cells (e.g., CD4+ cells). T cell responses include, for example, T cell cytotoxicity and proliferation. In some embodiments, the immune response (e.g., an anti-cancer immune response) is a cytotoxic T lymphocyte (CTL) response. As used herein, the term “cytotoxic T lymphocyte (CTL) response” refers to an immune response induced by cytotoxic T cells. CTL responses are mediated primarily by CD8+ T cells.

In one aspect, provided herein is a method for diagnosing or treating a condition associated with an antigen disclosed herein (e.g., CCR8). In some embodiments, the condition associated with CCR8 results due to an increase in the expression or biological activity of the CCR8. In some embodiments, the condition associated with CCR8 results due to a decrease in the expression or biological activity of the CCR8. In some embodiments, the methods of the disclosure comprise administering an effective amount of an antibody or a composition of the disclosure to a subject and/or contacting a population of cells (e.g., cells expressing CCR8, e.g., Treg cells) with an effective amount of an antibody or a composition of the disclosure. In some embodiments, the administering and/or contacting results in (a) binding of the antibody specifically to CCR8, (b) binding of the antibody to specifically to a cell expressing CCR8 (e.g., Treg or a cancer cell), (c) a decrease in expression of CCR8, (d) a decrease in a biological activity of CCR8, (e) a decrease in a CCR8 mediated signaling, (d) a decrease or an inhibition of binding of CCR8 to a CCL1 polypeptide, (e) inducing cytolysis (e.g., ADCC) of a cell expressing CCR8 (e.g., cytolysis of a Treg cell and/or a cancer cell), (f) a decrease in a level of cells expressing CCR8 (e.g., Treg cell or a cancer cell), (g) a decrease in a biological activity of a Treg cell, (h) inducing internalization of a CCR8 in a cell expressing CCR8 (e.g., internalization of CCR8 in a Treg cells, and/or a cancer cell), (i) enhancing an anti-cancer immune response or (j) or a combination thereof.

In some embodiments, the antibody disclosed herein is capable of (a) binding of the antibody specifically to CCR8, (b) binding of the antibody specifically to a cell expressing CCR8 (e.g., Treg or a cancer cell), (c) a decrease in expression of CCR8, (d) a decrease in a biological activity of CCR8, (e) a decrease in a CCR8 mediated signaling, (d) a decrease or an inhibition of binding of CCR8 to a CCL1 polypeptide, (e) inducing cytolysis (e.g., ADCC) of a cell expressing CCR8 (e.g., cytolysis of a Treg cell and/or a cancer cell), (f) a decrease in a level of cells expressing CCR8 (e.g., Treg cell or a cancer cell), (g) a decrease in a biological activity of a Treg cell, (h) inducing internalization of a CCR8 in a cell expressing CCR8 (e.g., internalization of CCR8 in a Treg cells, and/or a cancer cell), (i) enhancing an anti-cancer immune response or (j) or a combination thereof.

In some embodiments, the decrease is relative to a reference level. In some embodiments, the decrease is at least about 2, 5, 10, 15, 20, 25, 30, 35, 40, 45, 50, 55, 60, 65, 70, 75, 80, 85, 90, 91, 92, 93, 94, 95, 96, 97, 98, 99, 100% or more relative to a reference level. In some embodiments, the decrease is at least 2, 5, 10, 15, 20, 25, 30, 35, 40, 45, 50, 55, 60, 65, 70, 75, 80, 85, 90, 91, 92, 93, 94, 95, 96, 97, 98, 99, 100% or more relative to a reference level. In some embodiments, the reference level is a level in absence of the antibody disclosed herein. In some embodiments, the reference level is a level prior to administering of, contacting, and/or treatment with the antibody disclosed herein. In some embodiments, the reference level is a level in absence of administering of, contacting, and/or treatment with the antibody disclosed herein.

As used herein, “expression” may refer to transcription into a polynucleotide encoding a CCR8, translation into a CCR8 polypeptide, or even polynucleotide and/or polypeptide modifications (e.g., posttranslational modification of a polypeptide). Fragments of the transcribed polynucleotide encoding a CCR8, the translated polypeptide CCR8, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis.

“Reduced expression,” “reduced expression levels,” “reduced levels” or a “decrease in expression” can be a decrease in level of transcribed polynucleotide encoding a CCR8 (e.g., a mRNA encoding a CCR8), and/or CCR8 polypeptide, or even polynucleotide and/or polypeptide modifications (e.g., posttranslational modification of a polypeptide). In some embodiments, a decrease in expression is little or no expression. Expression level may be measured my methods known in the art such as RT-PCR, western blot, FACS.

In some embodiments, an antibody disclosed herein decreases a biological activity of CCR8 and/or a downstream pathway(s) i.e., a CCR8 mediated signaling or other CCR8-mediated function. In some embodiments, an antibody or an antigen binging fragment thereof is able to block, antagonize, suppress, inhibit or reduce a biological activity of CCR8. In some embodiments, the biological activity is binding of CCR8 to a ligand (e.g., Chemokine (C-C motif) ligand, activation of G protein signaling, CCR mediated signaling, induction of a cellular response (e.g., immune suppression).

In some embodiments, an antibody disclosed herein decreases binding of CCR8 to a ligand (e.g., CCL1), activation of G-protein signaling, including downstream pathways mediated by CCR8 signaling or function, such as receptor binding and/or induction of a cellular response to CCR8 or its metabolites (e.g., immune suppression). In some embodiments, an antibody disclosed herein binds to human CCR8 and/or prevents, blocks, inhibits or decreases binding of CCR8 to a ligand (e.g., CCL1). In some embodiments, an antibody disclosed herein binds to human CCR8 and/or prevents, blocks, inhibits or decreases interaction between CCR8 and G-protein. In some embodiments, an antibody disclosed herein prevents, blocks, inhibits or decreases the binding of CCR8 to CCL1. In some embodiments, an antibody disclosed herein prevents, blocks, inhibits or decreases the binding of CCR8 to CCL8. In some embodiments, an antibody disclosed herein prevents, blocks, inhibits or decreases the binding of CCR8 to CCL16. In some embodiments, an antibody disclosed herein prevents, blocks, inhibits or decreases the binding of CCR8 to CCL8. In some embodiments, an antibody disclosed herein prevents, blocks, inhibits or decreases the binding of CCR8 to CCL18.

In one aspect of the present disclosure, an antibody disclosed herein, can initiate a potent immune response against the cells expressing CCR8 and/or tumor cells by direct cytotoxicity. In this regard, an antibody disclosed herein may elicit cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. In addition, antibodies that exert a direct biological effect on tumor growth are useful in the practice of the disclosure. Potential mechanisms by which such directly cytotoxic antibodies may act include inhibition of cell growth, modulation of cellular differentiation, modulation of tumor angiogenesis factor profiles, and the induction of apoptosis. The mechanism by which a particular antibody disclosed herein, exerts an cytotoxic effect may be evaluated using any number of in vitro assays designed to determine ADCC, ADMMC, complement-mediated cell lysis, and so forth, as is generally known in the art.

The disclosure provides methods for treatment or prevention of a cancer, including, but not limited to, neoplasms, tumors, metastases, or any disease or disorder characterized by uncontrolled cell growth, by the administration of an antibody disclosed herein, to a patient in an amount effective to treat the patient.

In some embodiments, the cancer can be a carcinoma, a sarcoma, a lymphoma, a leukemia, germ cell tumor, a blastoma, or a melanoma. In some embodiments, the cancer can be a cancer from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In some embodiments, the cancer may be a neoplasm, malignant carcinoma, carcinoma, undifferentiated, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, adenocarcinoma; gastrinoma, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, Familial adenomatous polyposis, solid carcinoma, carcinoid tumor, branchiolo-alveolar adenocarcinoma, papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary and follicular adenocarcinoma, nonencapsulating sclerosing carcinoma, adrenal cortical carcinoma, endometroid carcinoma, skin appendage carcinoma, apocrine adenocarcinoma, sebaceous adenocarcinoma, ceruminous adenocarcinoma, mucoepidermoid carcinoma, cystadenocarcinoma, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, paget's disease, mammary acinar cell carcinoma, adenosquamous carcinoma, adenocarcinoma w/squamous metaplasia, thymoma, ovarian stromal tumor, thecoma, granulosa cell tumor, androblastoma, sertoli cell carcinoma, leydig cell tumor, lipid cell tumor, paraganglioma, extra-mammary paraganglioma, pheochromocytoma, glomangiosarcoma, melanoma, Lentigo maligna, Lentigo maligna melanoma, Acral lentiginous melanoma, mucosal melanoma, nodular melanoma, polypoid melanoma, desmoplastic melanoma, skin cutaneous melanoma, amelanotic melanoma, superficial spreading melanoma, melanoma in giant pigmented nevus, epithelioid cell melanoma, blue nevus, sarcoma, fibrosarcoma, fibrous histiocytoma, myxosarcoma, liposarcoma, leiomyosarcoma, rhabdomyosarcoma, embryonal rhabdomyosarcoma, alveolar rhabdomyosarcoma, stromal sarcoma, mixed tumor, mullerian mixed tumor, nephroblastoma, hepatoblastoma, carcinosarcoma, mesenchymoma, brenner tumor, phyllodes tumor, synovial sarcoma, mesothelioma, dysgerminoma, embryonal carcinoma, teratoma, struma ovarii, choriocarcinoma, mesonephroma, hemangiosarcoma, hemangioendothelioma, kaposi's sarcoma, hemangiopericytoma, lymphangiosarcoma, osteosarcoma, juxtacortical osteosarcoma, chondrosarcoma, chondroblastoma, mesenchymal chondrosarcoma, giant cell tumor of bone, ewing's sarcoma, odontogenic tumor, ameloblastic odontosarcoma, ameloblastoma, ameloblastic fibrosarcoma, pinealoma, chordoma glioma, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, neurofibrosarcoma, neurilemmoma, granular cell tumor, malignant lymphoma, hodgkin's disease, hodgkin's, paragranuloma, lymphoma, small lymphocytic, malignant lymphoma, Diffuse large B-cell lymphoma, follicular lymphoma, mycosis fungoides, other specified non-hodgkin's lymphomas, histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, or hairy cell leukemia. In some embodiments, the cancer is skin cancer. In some embodiments, the skin cancer is basal cell carcinoma, squamous cell carcinoma, cutaneous melanoma, merkel cell carcinoma, atypical fibroxanthoma, cutaneous lymphoma, or dermatofibrosarcoma. In some embodiments, the cutaneous melanoma is superficial spreading melanoma, nodular melanoma, acral lentiginous melanoma, subungual melanoma, lentigo maligna melanoma, desmoplastic melanoma, mucosal melanoma, or polypoid melanoma.

An antibody of the present disclosure may be administered to a subject per se or in the form of a pharmaceutical composition disclosed herein for the treatment or prevention of diseases, e.g., cancer. In some embodiments, an antibody or the compositions described herein may be administered alone or in combination with a second therapeutic agent or therapy useful for treating cancer. Examples of second therapy useful for treating cancer can include, but not limited to radiotherapy, cryotherapy, antibody therapy, chemotherapy, photodynamic therapy, surgery, hormonal therapy, immunotherapy, cytokine therapy, or a combination therapy with conventional drugs. In some embodiments, a second therapeutic agent, can be a cytotoxic drug, tumor vaccine, a peptide, a pepti-body, a small molecule, a cytotoxic agent, a cytostatic agent, immunological modifier, interferon, interleukin, immunostimulatory growth hormone, cytokine, vitamin, mineral, aromatase inhibitor, RNAi, Histone Deacetylase Inhibitor, proteasome inhibitor, a cancer chemotherapeutic agent, Tregs targeting agent, another antibody, Immunostimulatory antibody, a NSAID, a corticosteroid, a dietary supplement such as an antioxidant, cisplatin, ifosfamide, paclitaxel, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, and taxol. In some embodiments, the second therapeutic agent is a chemotherapeutic agent selected from a group consisting of platinum-based compounds, antibiotics with anti-cancer activity, Anthracyclines, Anthracenediones, alkylating agents, antimetabolites, Antimitotic agents, Taxanes, Taxoids, microtubule inhibitors, Vinca alkaloids, Folate antagonists, Topoisomerase inhibitors, Antiestrogens, Antiandrogens, Aromatase inhibitors, GnRh analogs, and inhibitors of 5α-reductase, biphosphonates.

In some embodiments, the second therapeutic agent can be a PD-1 inhibitor, histone deacetylase (HDAC) inhibitor, proteasome inhibitor, mTOR pathway inhibitor, JAK2 inhibitor, tyrosine kinase inhibitor (TKIs), PI3K inhibitor, Protein kinase inhibitor, Inhibitor of serine/threonine kinases, inhibitor of intracellular signaling, inhibitors of Ras/Raf signaling, MEK inhibitor, AKT inhibitor, inhibitor of survival signaling proteins, cyclin dependent kinase inhibitor, therapeutic monoclonal antibodies, TRAIL pathway agonist, anti-angiogenic agent, metalloproteinase inhibitor, cathepsin inhibitor, inhibitor of urokinase plasminogen activator receptor function, immunoconjugate, antibody drug conjugate, antibody fragments bispecfic antibodies, bispecific T cell engagers (BiTEs). In some embodiments, the another antibody is selected from cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab, aflibercept. In yet another embodiment, the second therapeutic agent can be antibodies currently used for the treatment of cancer. Examples of such antibodies include, but are not limited to, Herceptin®, Retuxan®, OvaRex, Panorex, BEC2, IMC-C225, Vitaxin, Campath I/H, Smart MI95, LymphoCide, Smart I D10, and Oncolym. In some embodiments, the antibody is an immunostimulatory antibody is selected from antagonistic antibodies targeting one or more of CTLA4, PD-1, PDL-1, LAG-3, TIM-3, BTLA, B7-H4, B7-H3, VISTA, and/or Agonistic antibodies targeting one or more of CD40, CD137, OX40, GITR, CD27, CD28, ICOS or a combination thereof. In some embodiments, the second therapeutic agent targeting immunosuppressive cells Tregs and/or MDSCs is selected from antimitotic drugs, cyclophosphamide, gemcitabine, mitoxantrone, fludarabine, thalidomide, thalidomide derivatives, COX-2 inhibitors, anti-CD25 daclizumab, basiliximab, ligand-directed toxins, denileukin diftitox (Ontak)—a fusion protein of human IL-2 and diphtheria toxin, or LMB-2—a fusion between an scFv against CD25 and the pseudomonas exotoxin, antibodies targeting Treg cell surface receptors, TLR modulators, agents that interfere with the adenosinergic pathway, ectonucleotidase inhibitors, or inhibitors of the A2A adenosine receptor, TGF-β inhibitors, chemokine receptor inhibitors, retinoic acid, all-trans retinoic acid (ATRA), Vitamin D3, phosphodiesterase 5 inhibitors, sildenafil, ROS inhibitors and nitroaspirin. In some embodiments, the second therapeutic agent is cytokine therapy selected from one or more of the following cytokines such as IL-2, IL-7, IL-12, IL-15, IL-17, IL-18 and IL-21, IL23, IL-27, GM-CSF, IFNα (interferon alpha), IFNα-2b, IFNβ, IFNγ, and their different strategies for delivery. In some embodiments, the second therapeutic agent is a therapeutic cancer vaccine selected from a group consisting of exogenous cancer vaccines including proteins or peptides used to mount an immunogenic response to a tumor antigen, recombinant virus and bacteria vectors encoding tumor antigens, DNA-based vaccines encoding tumor antigens, proteins targeted to dendritic cell-based vaccines, whole tumor cell vaccines, gene modified tumor cells expressing GM-CSF, ICOS and/or Flt3-ligand, oncolytic virus vaccines.

In some embodiments, the second therapeutic agent include EPO, G-CSF, ganciclovir; antibiotics, leuprolide; meperidine; zidovudine (AZT); interleukins 1 through 18, including mutants and analogues; interferons or cytokines, such as interferons a, ′ and y hormones, such as luteinizing hormone releasing hormone (LHRH) and analogues and, gonadotropin releasing hormone (GnRH); growth factors, such as transforming growth factor, fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-alpha (TNF-α); invasion inhibiting factor-2 (IIF-2); bone morphogenetic proteins 1-7 (BMP 1-7); somatostatin; thymosin-a-1; y-globulin; superoxide dismutase (SOD); complement factors; anti-angiogenesis factors; antigenic materials; and pro-drugs.

Prodrug refers to a precursor or derivative form of a pharmaceutically active substance that is less cytotoxic or non-cytotoxic to tumor cells compared to the parent drug and is capable of being enzymatically activated or converted into an active or the more active parent form. See, e.g., Wilman, “Prodrugs in Cancer Chemotherapy” Biochemical Society Transactions, 14, pp. 375-382, 615th Meeting Bel-fast (1986) and Stella et al., “Prodrugs: A Chemical Approach to Targeted Drug Delivery,” Directed Drug Delivery, Borchardt et al., (ed.), pp. 247-267, Humana Press (1985). Prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, lactam-containing prodrugs, optionally substituted phenoxyacetamide-containing prodrugs or optionally substituted phenylacetamide-containing prodrugs, 5-fluorocyto-sine and other 5-fluorouridine prodrugs which can be converted into the more active cytotoxic free drug. Examples of cytotoxic drugs that can be derivatized into a prodrug form for use herein include, but are not limited to, those chemotherapeutic agents described above.

In another embodiment, an antibody disclosed herein are administered, for the prevention or treatment of cancer prior to (e.g., 1 minute, 15 minutes, 30 minutes, 45 minutes, 1 hour, 2 hours, 4 hours, 6 hours, 8 hours, 12 hours, 24 hours, 2 days, or 1 week before), subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after), or concomitantly with the second therapeutic agent. In another embodiment, the second therapeutic agent can be antibodies immunospecific for one or more cancer cell antigens. In some embodiments, the cancer is refractory to other anti-cancer treatments or a second therapeutic agent. In some embodiments, the cancer is in remission. In some embodiments, one or more antibodies disclosed herein or are administered to an animal, preferably a mammal and most preferably a human. In some embodiments, the antibodies disclosed herein are administered after surgical resection of cancer. The method and compositions of the present disclosure contemplate single antibody disclosed herein, as well as combinations, or “cocktails”, of more than one antibody disclosed herein. In some embodiments, more than one antibody comprises at least 2, at least 3, at least 4, or all antibodies disclosed herein. Such antibody cocktails may have certain advantages inasmuch as they contain antibodies which exploit different effector mechanisms or combine directly cytotoxic antibodies with antibodies that rely on immune effector functionality.

### Antibody Based Gene Therapy

In another aspect of the disclosure, nucleic acids molecules comprising sequences encoding the antibodies of the disclosure, are administered to treat, inhibit or prevent a disease or disorder by way of gene therapy. In some embodiments, the disease is a cancer. In some embodiments, the disease is a skin cancer. In some embodiments, the disease is a skin cutaneous melanoma. In some embodiments, the disease is associated with aberrant expression and/or activity of an antigen that the antibody binds. Gene therapy refers to therapy performed by the administration to a subject of an expressed or expressible nucleic acid molecule. In this embodiment of the disclosure, the nucleic acid molecules produce their encoded protein (e.g., an antibody disclosed herein) that mediates a therapeutic effect. Any of the methods for gene therapy available can be used according to the present invention. Exemplary methods are described below. For general reviews of the methods of gene therapy, see Goldspiel et al., Clinical Pharmacy 12:488-505 (1993); Wu and Wu, Biotherapy 3:87-95 (1991).

In a one aspect, the nucleic acid molecule comprising nucleic acid sequences encoding an antibody, said nucleic acid molecule being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific.

In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijistra et al., Nature 342:435-438 (1989). In some embodiments, the expressed antibody is a single chain antibody; alternatively, the nucleic acid sequences include sequences encoding both the heavy and light chains, or fragments thereof, of the antibody.

Delivery of the nucleic acid molecules into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid molecules are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijistra et al., Nature 342:435-438 (1989)).

In a specific embodiment, viral vectors that contain nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid molecules encoding the antibody to be used in gene therapy are cloned into one or more vectors, which facilitates the delivery of the gene into a patient. More detail about retroviral vectors can be found in Boesen et al., Biotherapy 6:291-302 (1994).

Adenoviruses may also be used in the present invention. Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid molecule is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a one embodiment, the cell used for gene therapy is autologous to the patient. Nucleic acid sequences encoding an antibody of the present invention are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986).

### Dosages

Provided herein are compositions comprising antibodies disclosed herein for treatment (including prevention) of cancer. In some embodiments, the compositions are pharmaceutical compositions comprising a pharmaceutically acceptable carrier. The compositions are administered in an amount effective for treatment (including prophylaxis) of cancer. In some embodiments, the compositions (e.g., the antibodies or the nucleic acid molecules encoding said antibody) are administered in an amount effective for enhancing an immune response and/or increasing T cell activation in a subject. The compositions are to be used for in vivo administration to a subject by any available means, such as parenteral administration. For administration to a subject, a composition or medicament comprising the antibodies described herein can be sterile, which can readily be accomplished by filtration through sterile filtration membranes, or other methods known to those of skill in the art. In one embodiment, a composition or medicament has been treated to be free of pyrogens or endotoxins. Testing pharmaceutical compositions or medicaments for pyrogens or endotoxins and preparing pharmaceutical compositions or medicaments free of pyrogens or endotoxins or preparing pharmaceutical compositions or medicaments that have endotoxins at a clinically-acceptable level, are well understood to one of ordinary skill in the art. Commercial kits are available to test pharmaceutical compositions or medicaments for pyrogens or endotoxins.

The compositions to be used for in vivo administration, such as parenteral administration, in the methods described herein can be sterile, which is readily accomplished by filtration through sterile filtration membranes, or other methods known to those of skill in the art.

The antibodies disclosed herein, are formulated, dosed, and administered in a fashion consistent with good medical practice. Factors for consideration in this context include the particular disorder being treated, the particular subject being treated, the clinical condition of the individual subject, the cause of the disorder, the site of delivery of the agent, the method of administration, the scheduling of administration, and other factors known to medical practitioners. A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result. The “therapeutically effective amount” to be administered will be governed by such considerations, and refers to the minimum amount necessary to ameliorate, treat, or stabilize, the cancer; to increase the time until progression (duration of progression free survival) or to treat or prevent the occurrence or recurrence of a tumor, a dormant tumor, or a micrometastases. The antibodies disclosed herein, is optionally formulated with one or more additional therapeutic agents currently used to prevent or treat cancer or a risk of developing a cancer. The effective amount of such other agents depends on the amount of the antibody present in the formulation, the type of disorder or treatment, and other factors discussed above. These are generally used in the same dosages and with administration routes as used herein before or about from 1 to 99% of the heretofore employed dosage.

The dose of antibody may vary depending upon the age and the size of a subject to be administered, target disease, conditions, route of administration, and the like. The preferred dose is typically calculated according to body weight or body surface area. When an antibody disclosed herein is used for treating a condition or disease in an adult patient, it may be advantageous to intravenously administer an antibody of the present invention normally at a single dose of about 0.01 to about 20 mg/kg body weight, more preferably about 0.02 to about 7, about 0.03 to about 5, or about 0.05 to about 3 mg/kg, about 5 mg/kg, about 7.5 mg/kg, about 10 mg/kg, or about 15 mg/kg body weight. Depending on the severity of the condition, the frequency and the duration of the treatment can be adjusted. Effective dosages and schedules for administering may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

In some embodiments, the compositions herein can comprise a prophylactically effective amount, e.g., when administering to a subject at a risk of cancer or in earlier stages of a disease. A “prophylactically effective amount” refers to an amount effective, at dosages and for periods of time necessary, to achieve the desired prophylactic result. Typically, since a prophylactic dose is used in subjects prior to or at an earlier stage of disease, the prophylactic dose is lower than the therapeutic dose.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

The administration can be, for example, by one or more separate administrations, or by continuous infusion. For repeated administrations over several days or longer, depending on the condition, the treatment is sustained until, for example, the cancer is treated, as measured by the methods known in the art. However, other dosage regimens can be useful. In one non-limiting example, an antibody, disclosed herein is administered once every week, every two weeks, or every three weeks, at a dose range from about 5 mg/kg to about 15 mg/kg, including but not limited to 5 mg/kg, 7.5 mg/kg, 10 mg/kg or 15 mg/kg. The progress of using the methods described herein can be easily monitored by conventional techniques and assays. The duration of a therapy using the methods described herein will continue for as long as medically indicated or until a desired therapeutic effect (e.g., those described herein) is achieved. In certain embodiments, the administration of one or more antibodies, or compositions, described herein, is continued for 1 month, 2 months, 4 months, 6 months, 8 months, 10 months, 1 year, 2 years, 3 years, 4 years, 5 years, 10 years, 20 years, or for a period of years up to the lifetime of the subject.

### Efficacy of Treatment

The efficacy of the treatment methods for cancer, comprising administering the antibodies, or compositions (e.g., a pharmaceutical composition) of the present disclosure can be measured by various endpoints commonly used in evaluating cancer treatments, including but not limited to, tumor regression, tumor weight or size shrinkage, time to progression, duration of survival, progression free survival, overall response rate, duration of response, and quality of life. The antibodies disclosed herein can require unique measures and definitions of clinical responses to drugs. In the case of cancers, the therapeutically effective amount of the antibodies, disclosed herein or compositions comprising the same can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the antibodies disclosed herein, act to prevent growth and/or kill existing cancer cells; it can be cytostatic and/or cytotoxic. For cancer therapy, efficacy in vivo can, for example, be measured by assessing the duration of survival, duration of progression free survival (PFS), the response rates (RR), duration of response, and/or quality of life.

In those embodiments related to the treatment or prevention of skin cancer (e.g., cutaneous melanoma). Symptoms of melanoma include but are not limited to changes to the shape or color of existing moles or, in the case of nodular melanoma, the appearance of a new lump anywhere on the skin. At later stages, the mole may itch, ulcerate or bleed. Early signs of melanoma are summarized by asymmetry, borders (irregular with edges and corners), color (variegated), diameter (greater than 6 mm (0.24 in), about the size of a pencil eraser), evolving over time, funny looking. Nodular melanoma appears elevated above the skin surface, firm to the touch and growing. Metastatic melanoma may cause nonspecific paraneoplastic symptoms, including loss of appetite, nausea, vomiting and fatigue. Metastasis of early melanoma is possible, but relatively rare: less than a fifth of melanomas diagnosed early become metastatic. Brain metastases are particularly common in patients with metastatic melanoma. It can also spread to the liver, bones, abdomen or distant lymph nodes. In some embodiments, one or more symptoms of skin cancer (e.g., cutaneous melanoma) are inhibited or treated using the compositions and methods described herein.

In other embodiments, described herein are methods for increasing progression free survival of a human subject susceptible to or diagnosed with a cancer, for example, skin cancer, such as cutaneous melanoma. Time to disease progression is defined as the time from administration of the drug until disease progression or death. In a preferred embodiment, the combination treatment of the invention using an antibody disclosed herein, and one or more chemotherapeutic agents may significantly increase progression free survival by at least about 1 month, 1.2 months, 2 months, 2.4 months, 2.9 months, 3.5 months, such as by about 1 to about 5 months, when compared to a treatment with chemotherapy alone. In another embodiment, the methods described herein may significantly increase response rates in a group of human subjects susceptible to or diagnosed with a cancer that are treated with various therapeutics. Response rate is defined as the percentage of treated subjects who responded to the treatment. In one embodiment, the combination treatment described herein using an antibody disclosed herein, such as a recombinant antibody, and one or more chemotherapeutic agents significantly increases response rate in the treated subject group compared to the group treated with chemotherapy alone.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder including but not limited to, a chronic infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

For example, in some embodiments, the methods described herein comprise administering an effective amount of an antibody described herein, to a subject in order to alleviate a symptom of a cancer. As used herein, “alleviating a symptom of a cancer” is ameliorating or reducing any condition or symptom associated with the cancer. As compared with an equivalent untreated control, such reduction or degree of prevention is at least 5%, 10%, 20%, 40%, 50%, 60%, 80%, 90%, 95%, or 100% as measured by any standard technique. Ideally, the cancer is completely cleared as detected by any standard method known in the art, in which case the cancer is considered to have been treated. A patient who is being treated for a cancer is one who a medical practitioner has diagnosed as having such a condition. Diagnosis can be by any suitable means. Diagnosis and monitoring can involve, for example, detecting the level of cancer cells in a biological sample (for example, a tissue or lymph node biopsy, blood test, or urine test), detecting the level of a surrogate marker of the cancer in a biological sample, detecting symptoms associated with the specific cancer, or detecting immune cells involved in the immune response typical of such a cancer.

The term “effective amount” as used herein refers to the amount of an antibody or composition comprising the same needed to achieve a desired outcome including, but not limited to a decrease in a level of Treg cells, decrease in a biological activity of a Treg cell, enhance an immune response, and/or alleviate at least one or more symptom of the disease or disorder, and relates to a sufficient amount of pharmacological composition to provide the desired effect. The term “therapeutically effective amount” therefore refers to an amount of an antibody disclosed herein, that is sufficient to effect a particular effect when administered to a typical subject. An effective amount as used herein would also include an amount sufficient to delay the development of a symptom of the disease, alter the course of a symptom disease (for example but not limited to, slow the progression of a symptom of the disease), or reverse a symptom of the disease. Thus, it is not possible to specify the exact “effective amount”. For any given case, however, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50—Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the antibody), which achieves a half-maximal inhibition of symptoms as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The treatment and/or prevention of cancer includes, but is not limited to, alleviating symptoms associated with cancer, the inhibition of the progression of cancer, the promotion of the regression of cancer, the promotion of the immune response, inhibition of tumor growth, inhibition of tumor size, inhibition of metastasis, inhibition of cancer cell growth, inhibition of cancer cell proliferation, or cause cancer cell death.

### Modes of Administration

The antibodies described herein, can be administered to a subject in need thereof by any appropriate route which results in an effective treatment in the subject. As used herein, the terms “administering,” and “introducing” are used interchangeably and refer to the placement of an antibody or antibody portion thereof into a subject by a method or route which results in at least partial localization of such agents at a desired site, such as a site of infection, inflammation, or cancer, such that a desired effect(s) is produced.

In some embodiments, the antibodies described herein, or compositions comprising the same is administered to a subject having a cancer, to be inhibited by any mode of administration that delivers the agent systemically or to a desired surface or target, and can include, but is not limited to, injection, infusion, instillation, and inhalation administration. Oral administration forms are also contemplated herein. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intracranial, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the bispecific or multispecific polypeptide agent other than directly into a target site, tissue, or organ, such as a tumor site, such that it enters the subject's circulatory system and, thus, is subject to metabolism and other like processes.

In some embodiments, the antibodies, described herein, or compositions comprising the same can be administered via intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Local administration, for example, to a tumor or cancer site where angiogenesis is occurring, is particularly desired if extensive side effects or toxicity is associated with the use of the antibodies described herein, or compositions comprising the same. An ex vivo strategy can also be used for therapeutic applications in some embodiments. Ex vivo strategies involve transfecting or transducing cells obtained from a subject with a nucleic acid sequence, disclosed herein. The transfected or transduced cells are then returned to the subject. The cells can be any of a wide range of types including, without limitation, hematopoietic cells (e.g., bone marrow cells, macrophages, monocytes, dendritic cells, T cells, or B cells), fibroblasts, epithelial cells, endothelial cells, keratinocytes, or muscle cells.

In some embodiments, an antibody disclosed herein, or a composition comprising the same is administered by any suitable means, including parenteral, subcutaneous, intraperitoneal, intrapulmonary, and intranasal, and, if desired for local immunosuppressive treatment, intralesional administration.

Parenteral infusions include intramuscular, intravenous, intraarterial, intraperitoneal, or subcutaneous administration. In some embodiments, an antibody or compositions of the disclosure are suitably administered by pulse infusion, particularly with declining doses of the antibody. Preferably the dosing is given by injections, most preferably intravenous or subcutaneous injections, depending in part on whether the administration is brief or chronic. In some embodiments, an antibody or compositions of the disclosure are administered locally, e.g., by direct injections, when the disorder or location of the tumor permits, and the injections can be repeated periodically. In some embodiments, an antibody or compositions of the disclosure can also be delivered systemically to the subject or directly to the tumor cells, e.g., to a tumor or a tumor bed following surgical excision of the tumor, in order to prevent or reduce local recurrence or metastasis, for example of a dormant tumor or micrometastases.

Antibody-targeted sonoporation methods are contemplated for use in some embodiments of the methods for inhibiting tumors described herein, in order to enhance the efficacy and potency of the therapeutic compositions comprising an antibody provided herein. As used herein, “sonoporation” refers to the use of sound, preferably at ultrasonic frequencies, or the interaction of ultrasound with contrast agents (e.g., stabilized microbubbles) for temporarily modifying the permeability of cell plasma membranes, thus allowing uptake of large molecules, such as therapeutic agents. The membrane permeability caused by the sonoporation is transient, leaving the agents trapped inside the cell after the ultrasound exposure. Sonoporation employs acoustic cavitation of microbubbles to enhance delivery of large molecules.

Accordingly, in some embodiments of the methods, an antibody described herein, mixed with ultrasound contrast agents, such as microbubbles, can be injected locally or systemically into a subject in need of treatment for cancer, and ultrasound can be coupled and even focused into the defined area, e.g., tumor site, to achieve targeted delivery. In some embodiments, the methods use focused ultrasound methods to achieve targeted delivery. As used herein, HIFU or “High Intensity Focused Ultrasound” refers to a non-invasive therapeutic method using high-intensity ultrasound to heat and destroy malignant or pathogenic tissue without causing damage to overlying or surrounding health tissue. As described in Khaibullina et al., 49 J. Nucl. Med. 295 (2008), and WO 2010127369, HIFU can also be used as a means of delivery of therapeutic agents, such as antibodies or antibody fragments thereof.

Methods using contrast-enhanced ultrasound (CEUS) are also contemplated for use with an antibody described herein. Contrast-enhanced ultrasound (CEUS) refers to the application of ultrasound contrast medium and ultrasound contrast agents to traditional medical sonography. Ultrasound contrast agents refer to agents that rely on the different ways in which sound waves are reflected from interfaces between substances. A variety of microbubble contrast agents are available for use with the compositions and methods described herein. Microbubbles can differ in their shell makeup, gas core makeup, and whether or not they are targeted. Targeting ligands that bind to receptors characteristic of angiogenic disorders, can be conjugated to microbubbles, enabling the microbubble complex to accumulate selectively in areas of interest, such as diseased or abnormal tissues. This form of molecular imaging, known as targeted contrast-enhanced ultrasound, will only generate a strong ultrasound signal if targeted microbubbles bind in the area of interest. Targeted contrast-enhanced ultrasound has many applications in both medical diagnostics and medical therapeutics. In some embodiments, an antibody described herein, is administered to a subject in need of treatment for a cancer or a tumor, using a targeted ultrasound delivery.

### Diagnostic and Other Uses

Provided herein are methods of using the antibodies for detection, diagnosis and monitoring of a hCCR8 protein or a fragment thereof, cells expressing hCCR protein or a fragment thereof disease, disorder or condition associated with the antigen expression (either increased or decreased relative to a normal sample, and/or inappropriate expression, such as presence of expression in tissues(s) and/or cell(s) that normally lack the epitope expression), monitoring efficacy of a therapy for a disease, disorder or a condition such as cancer (e.g., immunotherapy). Provided herein are methods of determining whether a patient will respond to antibody therapy.

In some embodiments, the method comprises detecting whether the patient has cells that express target antigen (e.g., hCCR8) using an antibody disclosed herein. In some embodiments, the method of detection comprises contacting the sample with an antibody of the disclosure, and determining whether the level of binding differs from that of a reference or comparison sample (such as a control). In some embodiments, the method may be useful to determine whether the antibodies or polypeptides described herein are an appropriate treatment for the subject.

In some embodiments, the cells or cell/tissue lysate are contacted with an antibody and the binding between the antibody and the cell is determined. When the test cells show binding activity as compared to a reference cell of the same tissue type, it may indicate that the subject would benefit from treatment with an antibody. In some embodiments, the test cells are from human tissues. In some embodiments, the test cells are from human blood.

Various methods known in the art for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures.

Appropriate labels include, without limitation, radionuclides (for example 125I, 131I, 35S, 3H, or 32P), enzymes (for example, alkaline phosphatase, horseradish peroxidase, luciferase, or β-glactosidase), fluorescent moieties or proteins (for example, fluorescein, rhodamine, phycoerythrin, GFP, or BFP), or luminescent moieties (for example, Qdot™ nanoparticles supplied by the Quantum Dot Corporation, Palo Alto, Calif.). General techniques to be used in performing the various immunoassays noted above are known to those of ordinary skill in the art.

In some embodiments, (e.g., for purposes of diagnosis), the antibodies provided herein can be labeled with a detectable moiety including but not limited to radioisotopes, fluorescent labels, and various enzyme-substrate labels know in the art. Methods of conjugating labels to an antibody are known in the art.

In some embodiments, the antibodies need not be labeled, and the presence thereof can be detected using a second labeled antibody which binds to the first antibody. The antibodies of the present disclosure may be used as affinity purification agents for a cancer associated antigen or in diagnostic assays for a cancer associated antigen protein, e.g., detecting its expression in specific cells, tissues, or serum. The antibodies disclosed herein, may also be used for in vivo diagnostic assays. Generally, for these purposes the antibody is labeled with a radionuclide (such as u1In, 99Tc, 14C, 131I, 12sI, 3H, 32p or 3sS) so that the tumor can be localized using immunoscintiography.

The antibodies of the present invention may be employed in any known assay method, such as competitive binding assays, direct and indirect sandwich assays, such as ELISAs, and immunoprecipitation assays. Zola, Monoclonal Antibodies: A Manual of Techniques, pp. 147-158 (CRC Press, Inc. 1987). The antibodies may also be used for immunohistochemistry, to label tumor samples using methods known in the art. As a matter of convenience, an antibody of the present invention can be provided in a kit, i.e., a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### Kits

Provided herein are also kits, medicines, compositions, and unit dosage forms for use in any of the methods described herein. Provided herein is a kit comprising a therapeutically effective amount of at least one of the antibody disclosed herein. In some embodiments, the kit further comprises a second therapeutic agent (e.g., a chemotherapeutic agent). In some embodiments, the antibody is an aqueous form or a lyophilized form. The kit further comprises a diluent or a reconstitution solution.

Kits can include one or more containers comprising an antibody (or unit dosage forms and/or articles of manufacture). In some embodiments, a unit dosage is provided wherein the unit dosage contains a predetermined amount of a composition comprising an antibody (e.g., a therapeutically effective amount), with or without one or more additional agents. In some embodiments, such a unit dosage is supplied in single-use prefilled syringe for injection. In some embodiments, the composition comprising the antibody can comprise saline, sucrose, or the like; a buffer, such as phosphate, or the like; and/or be formulated within a stable and effective pH range. In some embodiments, the antibody can be provided as a lyophilized powder that may be reconstituted upon addition of an appropriate liquid, for example, sterile water. In some embodiments, the antibody further comprises one or more substances that inhibit protein aggregation, including, but not limited to, sucrose and arginine. In some embodiments, the antibody thereof further comprises heparin and/or a proteoglycan.

In some embodiments, kits further comprise instructions for use in the treatment of cancer in accordance with any of the methods described herein. The kit may further comprise a description of selection an individual suitable or treatment. Instructions supplied in the kits are typically written instructions on a label or package insert (for example, a paper sheet included in the kit), but machine-readable instructions (for example, instructions carried on a magnetic or optical storage disk) are also acceptable. In some embodiments, the kit further comprises another therapeutic agent (e.g., an anti-cancer antibody or a chemotherapeutic agent)

The kits are in suitable packaging. Suitable packaging includes, but is not limited to, vials, bottles, jars, flexible packaging (for example, sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### EXAMPLES

The following examples are provided in order to demonstrate and further illustrate certain preferred embodiments and aspects of the present invention and are not construed as limiting the scope thereof.

### Example 1 Anti-hCCR8 Antibody Generation

Due to low expression, conformational heterogeneity, and low stability, wild-type hCCR8 was unsuitable for use in VHH discovery. An EMP platform was used to evolve a stabilized version of hCCR8 (hCCR8-EMP) with improved properties. The hCCR8-EMP protein was expressed in HEK293 cells, purified, and formulated in apolipoprotein nanodiscs for use in llama immunization and subsequent phage library bio-panning coupled with next-generation sequencing to identify hits.

hCCR8-targeting single-domain antibodies were generated through hCCR8-EMP encoding DNA. Protein immunization of llamas with hCCR8-EMP DNA was performed as described in Pardon E., et al. (A general protocol for the generation of Nanobodies for structural biology, Nature Protocols, 2014, 9(3), 674-693) and Henry K. A. and MacKenzie C. R. eds. (Single-Domain Antibodies: Biology, Engineering and Emerging Applications. Lausanne: Frontiers Media), which is incorporated herein by reference in its entirety. Animals were immunized subcutaneously eight times at two-week intervals with 2 mg of DNA encoding the hCCR8-EMP inserted into the expression vector pVAX1 (ThermoFisher Scientific Inc., V26020), and 2 times with 500 micrograms of purified hCCR8-EMP protein. Blood samples were then obtained for peripheral blood mononuclear cells (PBMCs) isolation.

Phage display libraries derived from peripheral blood mononuclear cells (PBMCs) were prepared and used as described in Pardon E., et al. (A general protocol for the generation of Nanobodies for structural biology, Nature Protocols, 2014, 9(3), 674-693) and Henry K. A. and MacKenzie C. R. eds. (Single-Domain Antibodies: Biology, Engineering and Emerging Applications. Lausanne: Frontiers Media), which is incorporated herein by reference in its entirety. The VHH fragments were inserted into a M13 phagemid vector containing MYC and His6 tags (SEQ ID NO: 250). The libraries were rescued by infecting exponentially-growing Escherichia coli TG [(F′ traD36 proAB lacIqZ AM15) supE thi-1 A(lac-proAB) A(mcrB-hsdSM)5(rK− mK−)] cells followed by superinfection with the M13K07 helper phage (New England Biolabs).

Through the panning and hit characterization processes, more than 1000 unique VHH sequences were identified that could be placed in two unique major antibody families related to either VHH-1 (SEQ ID NO: 27) or VHH-2 (SEQ ID NO: 28) as determined through CDR3 sequence homology analysis.

### Example 2. Screening for hCCR8 Selection Outputs

Synthetic DNA fragments encoding hCCR8-binding VHHs were subcloned into an E. coli expression vector under control of an IPTG-inducible lac promoter, in frame with N-terminal PelB signal peptide for periplasmic compartment-targeting and C-terminal FLAG3 and His6 tags (SEQ ID NO: 250). Electrocompetent E. coli TG1 cells were transformed and the resulting clones were sequenced. VHH proteins were purified from these clones by IMAC chromatography followed by desalting, as described in Pardon E., et al. (A general protocol for the generation of Nanobodies for structural biology, Nature Protocols, 2014, 9(3), 674-693).

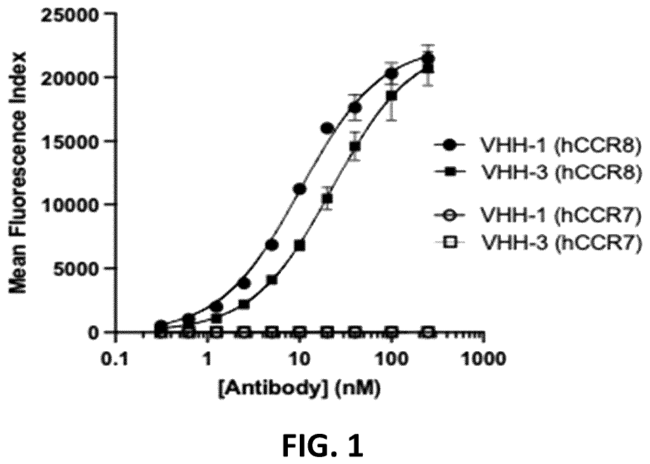

Recombinant cells expressing hCCR8 were resuspended to a final concentration of 1.0×10⁶ cells/ml in FACS buffer. Surface expression of hCCR8 on transiently transfected HEK293 cells was confirmed by flow cytometry of HEK293 cells stained with PE anti-hCCR8 (Clone 433H, BD Biosciences, #566897) antibody. Dilutions of purified VHHs were incubated with cells for 60 min. Individual samples were distributed into 96-well v-bottom plates and incubated for one hour at 4° C. Bound VHH were then detected with an anti-VHH specific antibody (Goat anti-alpaca VHH, Jackson ImmunoResearch, #128-605-232). FIG. 1 shows the binding analysis results of for two selected VHHs, namely VHH-1 (SEQ ID NO: 27) and VHH-3 (SEQ ID NO: 29). Binding was determined in parallel on HEK293 cells stably expressing the closely related receptor hCCR7 (SEQ ID NO: 41), as control for binding specificity.

### Example 3. Synthesis and Purification of VHH-Fc Fusions

Bivalent VHH-Fc fusions antibodies were constructed for VHH (SEQ ID NOs: 27, 28, and 70-86), identified from phage display biopanning efforts, by direct fusion of the VHH amino acid sequences to the short hinge of the human lgG1 Fc domain (SEQ ID NO: 34). These VHH-Fc fusions were constructed by cloning DNA encoding VHH domain into the antibody expression vector (pFUSE-hIgG1-Fc, Invivogen) that contained the coding sequence human IgG1 Fc domain using the manufacturer's protocol and standard molecular cloning methods. Examples of VHH-Fc fusion sequences for VHH-1 and VHH-2 can be found in the Table 3 as VHH-1-Fc (SEQ ID NO: 31) and VHH-2-Fc (SEQ ID NO: 33). VHH-Fc antibodies are named by the convention of (VHH name)-Fc, such that the VHH domain identity is readily identifiable from the antibody name. For example, VHH-1-Fc, (SEQ ID NO: 31), is comprised of the VHH domain, VHH-1 (SEQ ID NO: 27) fused to the short hinge of the human IgG1 Fc domain (SEQ ID NO: 34). From here forward, this naming convention will be used to describe fusion antibodies of various types. VHH-Fc antibodies were produced by DNA transient transfection into adherent HEK293 cells, expressed for 3 days and were purified by protein A purification using standard methods.

VHH-Fc fusion antibodies were evaluated for their ability to bind to hCCR8 on stably transfected HEK293 cells by means of flow cytometry. Cells were incubated with different concentrations of the bivalent VHH-Fc fusions for 60 minutes at 4° C., followed by two washes with FACS buffer, followed by 30 minutes incubation at 4° C. with the Zenon R-Phycoerythrin Anti-human IgG (ThermoFisher Scientific, #Z-25455), and two additional washing steps. Data were plotted and fit using GraphPad Prism software. EC50 and Bmax values individual antibodies were calculated from the resulting dose-response curves. Bivalent VHH-Fc fusions (Seq ID NOs: 31, 33, and 87-103) of 19 different VHH domains (SEQ ID NOs: 27, 28, and 70-86) were tested for cell binding and results are shown in Table 7. All VHH-Fc antibodies were found to bind with high affinity to hCCR8 expressed on HEK293 cells.

The anti-hCCR8 blocking monoclonal antibody, BMS-4A19 was used as control for the experiments described below. The sequence of BMS-4A19 was obtained by cloning the sequences of the complete light chain (SEQ ID NO: 36) and heavy chain variable (SEQ ID NO: 35) regions (corresponding respectively to SEQ ID NO: 102 and SEQ ID NO: 114 of WO2021194942A1) into an antibody expression vector. Production of this antibody was performed in HEK293 cells using standard methods. 4A19 was then expressed, purified, and validated for binding kinetics (0.3-0.6 nM on hCCR8 cells) as an assay comparator.

Representative cell binding curves for VHH-1-Fc (SEQ ID NO: 31), VHH-10-Fc, (SEQ ID NO: 92) VHH-11-Fc, (SEQ ID NO: 93), VHH-12-Fc (SEQ ID NO: 94), VHH-2-Fc (SEQ ID NO: 33) VHH-17-Fc (SEQ ID NO: 99), VHH-18-Fc (SEQ ID NO: 100) and BMS-4A19 are shown in FIG. 2A and FIG. 2B.

TABLE 4
Exemplary CDR sequences of the VHH
antibodies of the disclosure.

| SEQ | | SEQ | | SEQ | | SEQ | |
|---|---|---|---|---|---|---|---|
| ID | VHH | ID | | ID | | ID | |
| NO | name | NO | CDR1 | NO | CDR2 | NO | CDR3 |
| 27 | VHH-1 | 47 | GSTSSIRR | 48 | FSSGGGT | 49 | SARRLLGTSRVDY |
| 70 | VHH-5 | 47 | GSTSSIRR | 48 | FSSGGGT | 52 | NARRLLGTSRVDY |
| 71 | VHH-6 | 47 | GSTSSIRR | 48 | FSSGGGT | 53 | SARRLLGTSRYDY |
| 72 | VHH-7 | 47 | GSTSSIRR | 50 | FTSGGGT | 52 | NARRLLGTSRVDY |
| 73 | VHH-8 | 47 | GSTSSIRR | 51 | FSSGGST | 243 | SARRLLGLSRVDY |
| 74 | VHH-9 | 47 | GSTSSIRR | 50 | FTSGGGT | 53 | SARRLLGTSRYDY |
| 75 | VHH-10 | 47 | GSTSSIRR | 51 | FSSGGST | 244 | SARRLLGTSRVNY |
| 76 | VHH-11 | 47 | GSTSSIRR | 50 | FTSGGGT | 245 | SARRLLGTSRVGY |
| 77 | VHH-12 | 47 | GSTSSIRR | 50 | FTSGGGT | 246 | SARRLLGTSRVDS |
| 28 | VHH-2 | 54 | GSTSTINR | 55 | TSSRTSP | 56 | QARMAMGSSQVVY |
| 78 | VHH-13 | 54 | GSTSTINR | 55 | TSSRTSP | 61 | NARMAMGSSQVVY |
| 79 | VHH-14 | 54 | GSTSTINR | 59 | TSSGTSP | 62 | QARLAMGSSQVVY |
| 80 | VHH-15 | 54 | GSTSTINR | 60 | TSSRGSP | 63 | QARRAMGSSQVVY |
| 81 | VHH-16 | 54 | GSTSTINR | 60 | TSSRGSP | 64 | QARMTMGSSQVVY |
| 82 | VHH-17 | 54 | GSTSTINR | 55 | TSSRTSP | 65 | QARMAMGSSWVVY |
| 83 | VHH-18 | 57 | GSTSTINA | 55 | TSSRTSP | 66 | QARMAMGSSRVVY |
| 84 | VHH-19 | 58 | GSTSSINR | 55 | TSSRTSP | 67 | QARMAMGVSQVVY |
| 85 | VHH-20 | 54 | GSTSTINR | 55 | TSSRTSP | 68 | QARMAMGSSQVDY |
| 86 | VHH-21 | 54 | GSTSTINR | 60 | TSSRGSP | 69 | QARMAMASSQVVY |

TABLE 5
Exemplary amino acid sequences of the
VHH antibodies of the disclosure

| SEQ ID NO | Name | VHH Sequence |
|---|---|---|
| 70 | VHH-5 | QVQLQESGGGLVQAGGSLRLSCVASGSTSSIRRIGWYRQAPGKQR |
| | | ELVARFSSGGGTNYADFVKGRFTISGDNAKNTMYLQMNSLKPEDT |
| | | AVYYCNARRLLGTSRVDYWGQGTQVTVSS |
| 71 | VHH-6 | QVQLQESGGGLVQAGGSLRLSCVASGSTSSIRRIGWYRQAPGKQR |
| | | ELVARFSSGGGTNYADFVKGRFTISGDNAKNTMYLQMNSLKPEDT |
| | | AVYYCSARRLLGTSRYDYWGQGTQVTVSS |
| 72 | VHH-7 | QVQLQESGGGLVQAGGSLRLSCVASGSTSSIRRIGWYRQAPGKQR |
| | | ELVARFTSGGGTNYADFVKGRFTISGDNAKNTMYLQMNSLKPEDT |
| | | AVYYCNARRLLGTSRVDYWGQGTQVTVSS |
| 73 | VHH-8 | QVQLQESGGGLVQAGGSLRLSCVASGSTSSIRRIGWYRQAPGKQR |
| | | ELVARFSSGGSTNYADFVKGRFTISGDNAKNTMYLQMNSLKPEDT |
| | | AVYYCSARRLLGLSRVDYWGQGTQVTVSS |
| 74 | VHH-9 | QVQLQESGGGLVQAGGSLRLSCVASGSTSSIRRIGWYRQAPGKQR |
| | | ELVARFSSGGSTNYADFVKGRFTISGDNAKNTMYLQMNSLKPEDT |
| | | AVYYCSARRLLGTSRYDYWGQGTQVTVSS |
| 75 | VHH- | QVQLQESGGGLVQAGGSLRLSCVASGSTSSIRRIGWYRQAPGKQR |
| | 10 | ELVARFSSGGSTNYADFVKGRFTISGDNAKNTMYLQMNSLKPEDT |
| | | AVYYCSARRLLGTSRVNYWGQGTQVTVSS |
| 76 | VHH- | QVQLQESGGGLVQAGGSLRLSCVASGSTSSIRRIGWYRQAPGKQR |
| | 11 | ELVARFTSGGGTNYADFVKGRFTISGDNAKNTMYLQMNSLKPEDT |
| | | AVYYCSARRLLGTSRVGYWGQGTQVTVSS |
| 77 | VHH- | QVQLQESGGGLVQAGGSLRLSCVASGSTSSIRRIGWYRQAPGKQR |
| | 12 | ELVARFTSGGGTNYADFVKGRFTISGDNAKNTMYLQMNSLKPEDT |
| | | AVYYCSARRLLGTSRVDSWGQGTQVTVSS |
| 78 | VHH- | QVQLQESGGGLVQAGGSLRLTCAVSGSTSTINRFAWYRQAPGKQR |
| | 13 | ELVAATSSRTSPNYADSVKGRFRFSRDNARSAVYLQMDNLRPEDT |
| | | AVYFCNARMAMGSSQVVYWGLGTQVTVSS |
| 79 | VHH- | QVQLQESGGGLVQAGGSLRLTCAVSGSTSTINRFAWYRQAPGKQR |
| | 14 | ELVAATSSGTSPNYADSVKGRFRFSRDNARSAVYLQMDNLRPEDT |
| | | AVYFCQARLAMGSSQVVYWGLGTQVTVSS |
| 80 | VHH- | QVQLQESGGGLVQAGGSLRLTCAVSGSTSTINRFAWYRQAPGKQR |
| | 15 | ELVAATSSRGSPNYADSVKGRFRFSRDNARSAVYLQMDNLRPEDT |
| | | AVYFCQARRAMGSSQVVYWGLGTQVTVSS |
| 81 | VHH- | QVQLQESGGGLVQAGGSLRLTCAVSGSTSTINRFAWYRQAPGKQR |
| | 16 | ELVAATSSRGSPNYADSVKGRFRFSRDNARSAVYLQMDNLRPEDT |
| | | AVYFCQARMTMGSSQVVYWGLGTQVTVSS |
| 82 | VHH- | QVQLQESGGGLVQAGGSLRLTCAVSGSTSTINRFAWYRQAPGKQR |
| | 17 | ELVAATSSRTSPNYADSVKGRFRFSRDNARSAVYLQMDNLRPEDT |
| | | AVYFCQARMAMGSSWVVYWGLGTQVTVSS |
| 83 | VHH- | QVQLQESGGGLVQAGGSLRLTCAVSGSTSTINAFAWYRQAPGKQR |
| | 18 | ELVAATSSRTSPNYADSVKGRFRFSRDNARSAVYLQMDNLRPEDT |
| | | AVYFCQARMAMGSSRVVYWGLGTQVTVSS |
| 84 | VHH- | QVQLQESGGGLVQAGGSLRLTCAVSGSTSSINRFAWYRQAPGKQR |
| | 19 | ELVAATSSRTSPNYADSVKGRFRFSRDNARSAVYLQMDNLRPEDT |
| | | AVYFCQARMAMGVSQVVYWGLGTQVTVSS |
| 85 | VHH- | QVQLQESGGGLVQAGGSLRLTCAVSGSTSTINRFAWYRQAPGKQR |
| | 20 | ELVAATSSRTSPNYADSVKGRFRFSRDNARSAVYLQMDNLRPEDT |
| | | AVYFCQARMAMGSSQVDYWGLGTQVTVSS |
| 86 | VHH- | QVQLQESGGGLVQAGGSLRLTCAVSGSTSTINRFAWYRQAPGKQR |
| | 21 | ELVAATSSRGSPNYADSVKGRFRFSRDNARSAVYLQMDNLRPEDT |
| | | AVYFCQARMAMASSQVVYWGLGTQVTVSS |

TABLE 6
Exemplary amino acid sequences of the
VHH domains of the disclosure fused
to a Fc region (VHH-Fc Antibodies)

| SEQ ID | VHH- Fc | |
|---|---|---|
| NO | Name | VHH-Fc Sequence |
| 87 | VHH- | QVQLQESGGGLVQAGGSLRLSCVASGSTSSIRRIGWYRQAPGKQR |
| | 5-Fc | ELVARFSSGGGTNYADFVKGRFTISGDNAKNTMYLQMNSLKPEDT |
| | | AVYYCNARRLLGTSRVDYWGQGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 88 | VHH- | QVQLQESGGGLVQAGGSLRLSCVASGSTSSIRRIGWYRQAPGKQR |
| | 6-Fc | ELVARFSSGGGTNYADFVKGRFTISGDNAKNTMYLQMNSLKPEDT |
| | | AVYYCSARRLLGTSRYDYWGQGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 89 | VHH- | QVQLQESGGGLVQAGGSLRLSCVASGSTSSIRRIGWYRQAPGKQR |
| | 7-Fc | ELVARFTSGGGTNYADFVKGRFTISGDNAKNTMYLQMNSLKPEDT |
| | | AVYYCNARRLLGTSRVDYWGQGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 90 | VHH- | QVQLQESGGGLVQAGGSLRLSCVASGSTSSIRRIGWYRQAPGKQR |
| | 8-Fc | ELVARFSSGGSTNYADFVKGRFTISGDNAKNTMYLQMNSLKPEDT |
| | | AVYYCSARRLLGLSRVDYWGQGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 91 | VHH- | QVQLQESGGGLVQAGGSLRLSCVASGSTSSIRRIGWYRQAPGKQR |
| | 9-Fc | ELVARFSSGGSTNYADFVKGRFTISGDNAKNTMYLQMNSLKPEDT |
| | | AVYYCSARRLLGTSRYDYWGQGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 92 | VHH- | QVQLQESGGGLVQAGGSLRLSCVASGSTSSIRRIGWYRQAPGKQR |
| | 10-Fc | ELVARFSSGGSTNYADFVKGRFTISGDNAKNTMYLQMNSLKPEDT |
| | | AVYYCSARRLLGTSRVNYWGQGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 93 | VHH- | QVQLQESGGGLVQAGGSLRLSCVASGSTSSIRRIGWYRQAPGKQR |
| | 11-Fc | ELVARFTSGGGTNYADFVKGRFTISGDNAKNTMYLQMNSLKPEDT |
| | | AVYYCSARRLLGTSRVGYWGQGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 94 | VHH- | QVQLQESGGGLVQAGGSLRLSCVASGSTSSIRRIGWYRQAPGKQR |
| | 12-Fc | ELVARFTSGGGTNYADFVKGRFTISGDNAKNTMYLQMNSLKPEDT |
| | | AVYYCSARRLLGTSRVDSWGQGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 95 | VHH- | QVQLQESGGGLVQAGGSLRLTCAVSGSTSTINRFAWYRQAPGKQR |
| | 13-Fc | ELVAATSSRTSPNYADSVKGRFRFSRDNARSAVYLQMDNLRPEDT |
| | | AVYFCNARMAMGSSQVVYWGLGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 96 | VHH- | QVQLQESGGGLVQAGGSLRLTCAVSGSTSTINRFAWYRQAPGKQR |
| | 14-Fc | ELVAATSSGTSPNYADSVKGRFRFSRDNARSAVYLQMDNLRPEDT |
| | | AVYFCQARLAMGSSQVVYWGLGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 97 | VHH- | QVQLQESGGGLVQAGGSLRLTCAVSGSTSTINRFAWYRQAPGKQR |
| | 15-Fc | ELVAATSSRGSPNYADSVKGRFRFSRDNARSAVYLQMDNLRPEDT |
| | | AVYFCQARRAMGSSQVVYWGLGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 98 | VHH- | QVQLQESGGGLVQAGGSLRLTCAVSGSTSTINRFAWYRQAPGKQR |
| | 16-Fc | ELVAATSSRGSPNYADSVKGRFRFSRDNARSAVYLQMDNLRPEDT |
| | | AVYFCQARMTMGSSQVVYWGLGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 99 | VHH- | QVQLQESGGGLVQAGGSLRLTCAVSGSTSTINRFAWYRQAPGKQR |
| | 17-Fc | ELVAATSSRTSPNYADSVKGRFRFSRDNARSAVYLQMDNLRPEDT |
| | | AVYFCQARMAMGSSWVVYWGLGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 100 | VHH- | QVQLQESGGGLVQAGGSLRLTCAVSGSTSTINAFAWYRQAPGKQR |
| | 18-Fc | ELVAATSSRTSPNYADSVKGRFRFSRDNARSAVYLQMDNLRPEDT |
| | | AVYFCQARMAMGSSRVVYWGLGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 101 | VHH- | QVQLQESGGGLVQAGGSLRLTCAVSGSTSSINRFAWYRQAPGKQR |
| | 19-Fc | ELVAATSSRTSPNYADSVKGRFRFSRDNARSAVYLQMDNLRPEDT |
| | | AVYFCQARMAMGVSQVVYWGLGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 102 | VHH- | QVQLQESGGGLVQAGGSLRLTCAVSGSTSTINRFAWYRQAPGKQR |
| | 20-Fc | ELVAATSSRTSPNYADSVKGRFRFSRDNARSAVYLQMDNLRPEDT |
| | | AVYFCQARMAMGSSQVDYWGLGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |
| 103 | VHH- | QVQLQESGGGLVQAGGSLRLTCAVSGSTSTINRFAWYRQAPGKQR |
| | 21-Fc | ELVAATSSRGSPNYADSVKGRFRFSRDNARSAVYLQMDNLRPEDT |
| | | AVYFCQARMAMASSQVVYWGLGTQVTVSSDKTHTCPPCPAPELLG |
| | | GPSVFLFPPKPKDTLMISRTPEVTCVVVDVSHEDPEVKFNWYVDG |
| | | VEVHNAKTKPREEQYNSTYRVVSVLTVLHQDWLNGKEYKCKVSNK |
| | | ALPAPIEKTISKAKGQPREPQVYTLPPSRDELTKNQVSLTCLVKG |
| | | FYPSDIAVEWESNGQPENNYKTTPPVLDSDGSFFLYSKLTVDKSR |
| | | WQQGNVFSCSVMHEALHNHYTQKSLSLSPGK |

Table 7 shows that VHH-Fc antibodies displayed potent hCCR8 binding as indicated by EC50 values<5 nM and a normalized Bmax relative to VHH-1-Fc or VHH-2-Fc of >50%.

TABLE 7
Binding of VHH-Fc Antibodies to cells expressing hCCR8

| | VHH-Fc | EC50 | |
|---|---|---|---|
| Clone Name | SEQ ID NO | (nM) | Bmax % |
| VHH-1-Fc | 31 | 2.97 | 100 |
| VHH-5-Fc | 87 | 3.29 | 104.3 |
| VHH-6-Fc | 88 | 3.10 | 65.8 |
| VHH-7-Fc | 89 | 4.07 | 104.9 |
| VHH-8-Fc | 90 | 4.19 | 99.7 |
| VHH-9-Fc | 91 | 4.35 | 71.1 |
| VHH-10-Fc | 92 | 1.44 | 76.4 |
| VHH-11-Fc | 93 | 1.82 | 63.5 |
| VHH-12-Fc | 94 | 1.72 | 91.8 |
| VHH-2-Fc | 33 | 1.24 | 100 |
| VHH-13-Fc | 95 | 1.38 | 51.8 |
| VHH-14-Fc | 96 | 4.28 | 85.6 |
| VHH-15-Fc | 97 | 1.16 | 95.4 |
| VHH-16-Fc | 98 | 1.10 | 69.5 |
| VHH-17-Fc | 99 | 1.54 | 80.4 |
| VHH-18-Fc | 100 | 1.35 | 70.7 |
| VHH-19-Fc | 101 | 0.66 | 72.0 |
| VHH-20-Fc | 102 | 3.26 | 72.1 |
| VHH-21-Fc | 103 | 1.58 | 69.1 |

### Example 4. Epitope Mapping

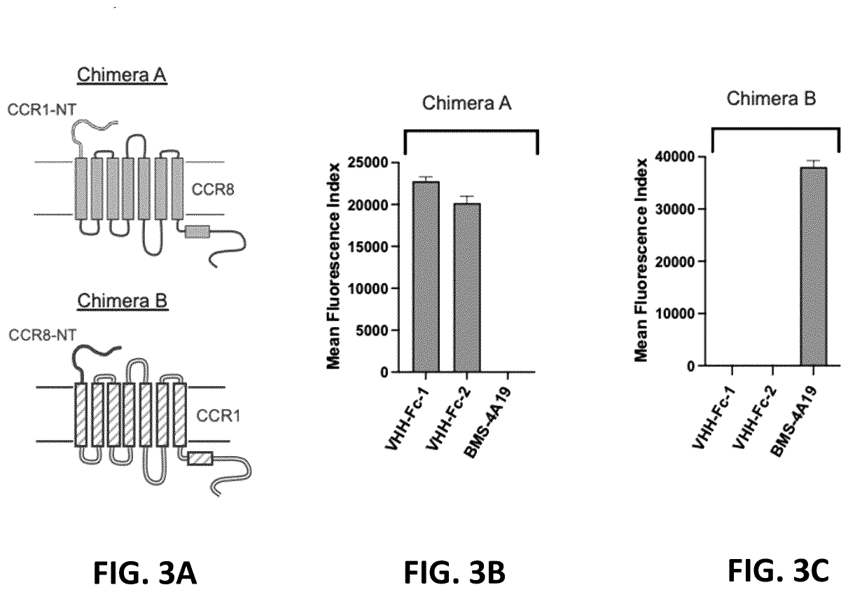

Determination of binding epitope location on hCCR8 was done for representative VHH-Fc antibodies by flow cytometry analysis using HEK293T cells expressing hCCR8 or two receptor chimeras comprised of combined sequence elements taken from hCCR8 (SEQ ID NO: 37) and hCCR1 (SEQ ID NO: 39). In the first, Chimera A based on hCCR8, the N-terminal region of the receptor (SEQ ID NO: 38) was replaced with a sequence encoding the N-terminal region of hCCR1 (SEQ ID NO: 40). In Chimera B based on hCCR1, the N-terminal region of the receptor was replaced by the sequence encoding the hCCR8 N-terminus (SEQ ID NO: 38). Drawings of the receptor chimera constructs are shown in FIG. 3A. Comparison of the binding signal (MFI) of a given VHH-Fc clone upon binding to cells expressing hCCR8, Chimera A (FIG. 3B) and Chimera B (FIG. 3C), allowed the localization of the antibody binding epitope to either the hCCR8 transmembrane domain, or the N-terminal region. Using this approach, we were able to establish that VHH-1-Fc (SEQ ID NO: 31) and VHH-2-Fc (SEQ ID NO: 33) bound exclusively to the hCCR8 TMD, while BMS-4A19 bound only to the N-terminus.

### Example 5. Characterization of Antibody Binding Interactions

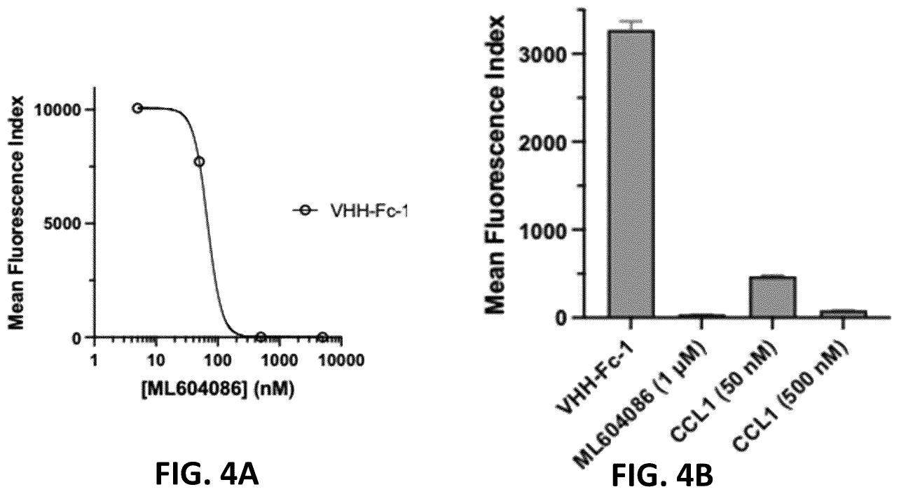

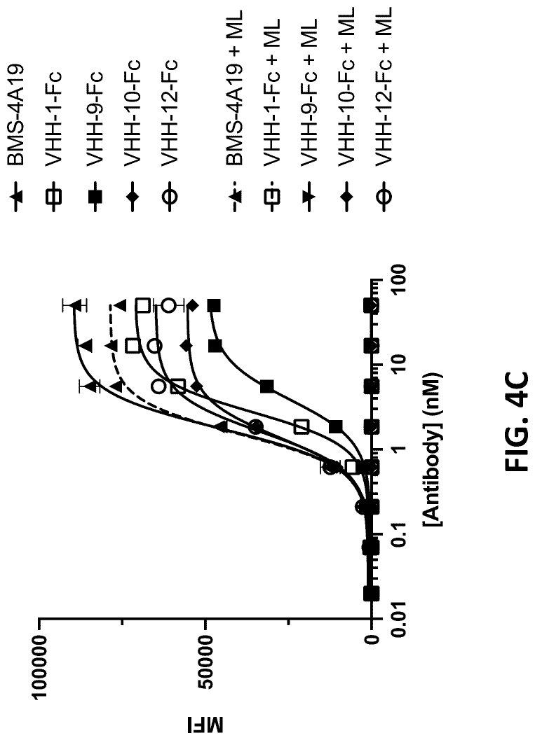

VHH-Fc fusions were evaluated for their ability to bind to hCCR8 expressed on cells in the presence and absence of either the chemokine antagonist CCL1 or the potent small molecule antagonist ML604086. HEK293 cells expressing hCCR8 were incubated with a fixed concentration (10 nM) of VHH-1-Fc (SEQ ID NO: 31) in the presence of different concentrations ML604086 (FIG. 4A) or in a separate experiment, using fixed concentrations of either ML604086 or CCL1 (FIG. 4B). Binding studies were conducted on hCCR8 expressing cells for 60 minutes at 4° C., followed by two washes and then 30 minutes incubation at 4° C. with the Zenon R-Phycoerythrin Anti-human IgG secondary antibody (ThermoFisher Scientific, #Z-25455). Binding to cells was assessed by flow cytometry. ML604086 and CCL1 were able to completely prevent binding to hCCR8 at the highest concentrations, indicating that VHH-1-Fc interacted at a site overlapping the receptor's antagonist binding pocket. The VHH-Fc clones, VHH-1-Fc (SEQ ID NO: 31), VHH-9-Fc (SEQ ID NO: 91), VHH-10-Fc (SEQ ID NO: 92) and VHH-12-Fc (SEQ ID NO: 94) were also tested for binding to HEK293 cells expressing hCCR8 as described above, in the presence or absence of 10 μM added ML604086 (FIG. 4C). Antibodies were shown to bind with high affinity, but cell binding was completely blocked by ML604086, indicating a similar binding mode and high specificity for hCCR8 as was seen initially for VHH-1 (SEQ ID NO: 27) and its derivative Fc fusion antibody, VHH-1-Fc (SEQ ID NO: 31). The N-terminal hCCR8 binding antibody BMS-4A19 was unaffected by added ML604086, showing that the effect was unique to the transmembrane domain binding VHH antibodies of the invention.

### Example 6. Characterization of Antibody Pharmacology

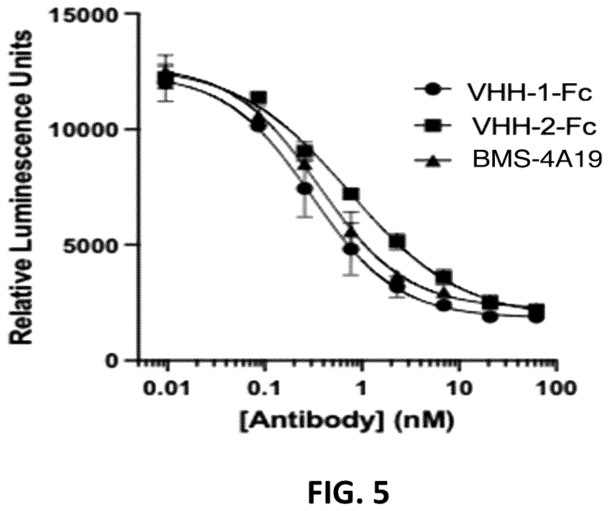

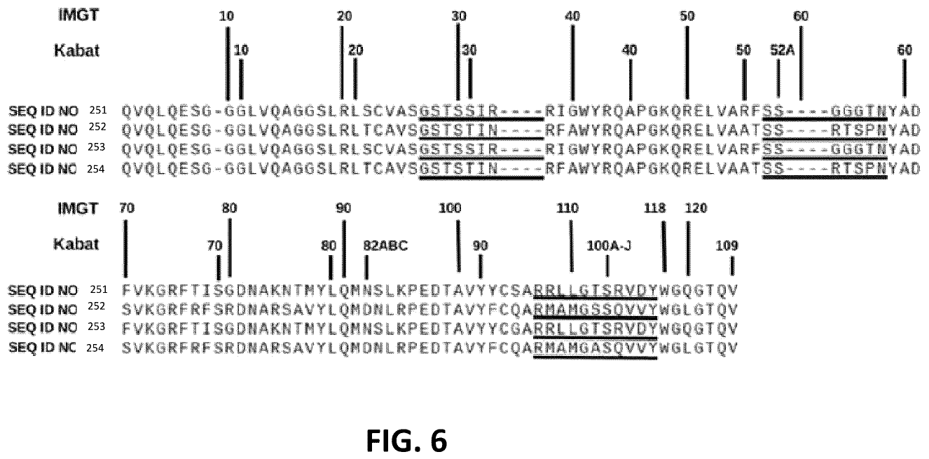

Agonist-induced internalization of hCCR8 and other GPCRs is a regulatory mechanism often driven by engagement of the activated receptor by β-arrestin. Antagonism of this pathway is desirable to prevent the loss of bound anti-CCR8 antibodies from the cell surface with a concomitant loss efficacy. We characterized VHH-Fc molecules for the inhibition of CCL1-induced β-arrestin recruitment (DiscoverX PathHunter® eXpress, Eurofins) (FIG. 5). A fixed concentration (EC80) of the hCCR8 agonist CCL1 (R&D Systems, #272-I) was used to stimulate receptor β-arrestin recruitment. Multipoint dilution series of the VHH-Fc molecules were mixed with the assay components to inhibit CCL1 activation of CCR8. Inhibition dose-response curves were plotted using from which the concentration necessary to achieve 50% inhibition (IC50 could be determined. BMS-4A19 was included as a positive control for inhibition. In this assay the VHH-1-Fc (SEQ ID NO: 31) and VHH-2-Fc (SEQ ID NO: 33) were found to be potent antagonists with sub-nanomolar IC50 values.

### Example 7. Evaluation of VHH-Fc ADCC Effector Activity

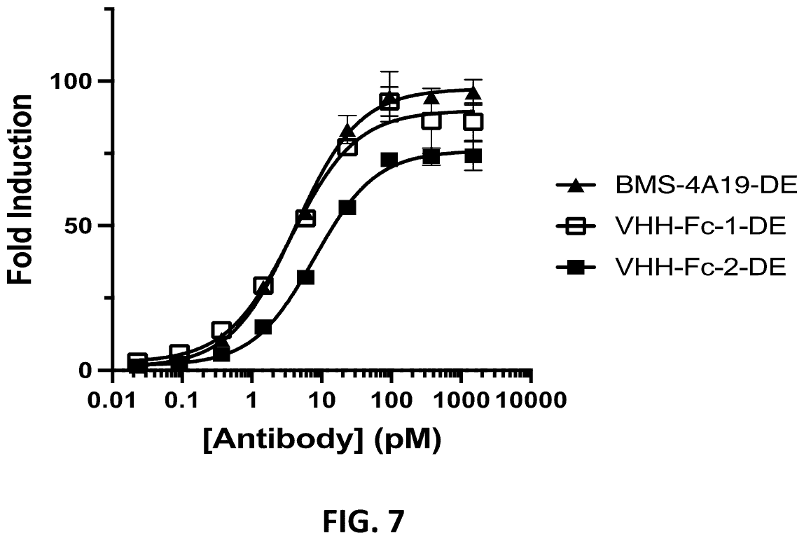

VHH-Fc fusions were tested for their capacity to activate either the human FcγRIIIa or the mouse FcγR1V in the ADCC reporter assay (Promega, G7010, G7018). In this assay engineered Jurkat cells are used as effector cells that are stably transfected with the V158 FcγRIIIa receptor and an NFAT (nuclear factor of activated T-cells) responsive firefly luciferase reporter gene. HuT-78 cells that naturally express human CCR8 were used as target cells. ADCC activity was quantified through the produced luciferase luminescence signal resulting from the NFAT pathway activation upon incubation of the VHH-Fc fusions with the target and effector cells at a 2.5:1 effector:target cell ratio, according to the recommendations of the manufacturer. This assay design was used to determine the effector function of human IgG1 chimera and humanized versions of anti-CCR8. Briefly, Hut-78 cells were resuspended in prewarmed assay buffer (37° C.) at 10⁶ cells/mL concentration. 25,000 cells were mixed with serially diluted anti-CCR8 antibodies in 96-well flat, clear-bottom plate, and then incubated for 1 hour at 37° C., 5% CO₂. Promega BioAssay effector cells were added to individual well at varied ratios to target cells and further incubated for 6 hours at 37° C. 5% CO₂. After incubation, assay plates were equilibrated to room temperature for 15 min under foil on the benchtop. Pre-mixed Bio-Glo luciferase assay substrate was added to each well and incubated at room temperature for 5 min. The assay plates were then read on a Tecan plate reader within 30 min of substrate addition. Data were plotted and fit using GraphPad Prism software and EC50 and Bmax values individual antibodies were calculated from the resulting dose-response curves. Results of the ADCC assay for VHH-1-Fc-DE (SEQ ID NO: 45), VHH-2-Fc-DE (SEQ ID NO: 46) and the BMS-4A19-DE antibody (SEQ ID NO: 36 and SEQ ID NO: 240) are shown in FIG. 7. BMS-4A19-DE, VHH-1-Fc-DE and VHH-2-Fc-DE were potent activators in the ADCC assay, with EC50 values of 4.1 pM, 3.7 pM and 7.9 pM respectively.

### Example 8. Humanization of VHH Framework Sequence

In order to optimize VHH domains to reduce potential immunogenicity of VHH domains in humans upon therapeutic administration, VHH-1 (SEQ ID NO: 27) and VHH-2 (SEQ ID NO: 27) sequences were humanized by substitution of residues within framework positions (i.e., non-CDR residues). The amino acid sequences of VHH-1 and VHH-2 were compared to a database of known human germline sequences from human VH genes (IMGT® the international ImMunoGeneTics information System® www.imgt.org). The databases used were for comparison were the IMGT human VH genes (F+ORF) and were analyzed for similarity by the NCBI IgBLAST program. Using these methods, the closest human germline sequences to both VHH-1 and VHH-2 were the IGHV3-23(allele 4) (SEQ ID NO: 104 or 255) and the human heavy chain IGHJ6(allele 1) (SEQ ID NO: 105) joining region (J gene). Residues differing between the human germline sequences and the parental llama VHH-1 or VHH-2 framework sequences were identified (non-CDR residues in the human VH3 sequence). Using this information several constructs were generated where residues at equivalent framework positions within VHH-1 (SEQ ID NO: 27) or VHH-2 (SEQ ID NO: 28) were substituted to varying degrees to match the human framework region to account for potential changes in properties of the humanized VHH antibody relative to the parent. Framework amino acid positions were changed in VHH-1 or VHH-2 to generate multiple humanized versions of VHH-1 or VHH-2, while maintaining the original VHH-1 or VHH-2 CDRs unchanged according to CDR boundaries defined by the IMGT definition. Changes made within for each engineered sequence are noted in FIG. 8A for VHH-1 and in FIG. 8B for VHH-2. Humanization of VHH-1 framework residues was performed to generate 3 variants for VHH-1 and VHH-2 that contain substitutions relative to the parental llama framework residues to match the human IGHV3-23 framework to varying degrees. Humanized VHH coding sequences were constructed by synthetic DNA synthesis (Twist Bioscience). The most humanized variant of VHH-1, VHH-1.1 (SEQ ID NO: 106) contains a total of 17 of 19 possible humanizing substitutions in the framework regions (see asterisks on sequence alignment, FIG. 8A), while VHH-1.2 (SEQ ID NO: 107) contains 12 framework substitutions and VHH-1.3 (SEQ ID NO: 108) contains 10 substitutions. The three humanized versions of VHH-2 are VHH-2.1 (SEQ ID NO: 109), VHH-2.2 (SEQ ID NO: 110), and VHH-2.3 (SEQ ID NO: 111), and contain 21, 13 and 11 substitutions, respectively. Humanized VHH sequences are shown in Table 8 and humanized framework regions (FR1-FR4) are shown in Table 9 along with the corresponding parental VHH-1 and VHH-2 framework regions.

SEQ ID NO: 104—IGHV3-23*04—Amino acid sequence of heavy chain human germline

EVQLVESGGGLVQPGGSLRLSCAASGFTFSSYAMSWVRQAPGKGLEWVSAISGSGGSTY YADSVKGRFTISRDNSKNTLYLQMNSLRAEDTAVYYCAK (SEQ ID NO: 104).

SEQ ID NO: 105—GHJ6*01—Amino acid sequence ofhuman germlSe

YYYYYGMDVWGQGTTVTVSS (SEQ ID NO: 105).

TABLE 8
Exemplary amino acid sequences of humanized antibodies
(e.g., VHH antibodies) of the disclosure

| SEQ ID | | | |
|---|---|---|---|
| NO | Name | Description | |
| 106 | VHH-1.1 | Amino acid | EVQLVESGGGLVQPGGSLRLSCAASGSTSSIRRI |
| | | sequence of | GWVRQAPGKGLEWVSRFSSGGGTNYADSVKGRFT |
| | | humanized | ISRDNSKNTLYLQMNSLRAEDTAVYYCAKRRLLG |
| | | VHH-1, | TSRVDYWGQGTTVTVSS |
| | | version 1 | |
| 107 | VHH-1.2 | Amino acid | EVQLVESGGGLVQPGGSLRLSCAASGSTSSIRRI |
| | | sequence of | GWYRQAPGKGLEWVARFSSGGGTNYADSVKGRFT |
| | | humanized | ISGDNSKNTMYLQMNSLRAEDTAVYYCSARRLLG |
| | | VHH-1, | TSRVDYWGQGTTVTVSS |
| | | version 2 | |
| 108 | VHH-1.3 | Amino acid | EVQLVESGGGLVQPGGSLRLSCAASGSTSSIRRI |
| | | sequence of | GWYRQAPGKGRELVARFSSGGGTNYADSVKGRFT |
| | | humanized | ISGDNSKNTMYLQMNSLRAEDTAVYYCSARRLLG |
| | | VHH-1, | TSRVDYWGQGTTVTVSS |
| | | version 3 | |
| 109 | VHH-2.1 | Amino acid | EVQLVESGGGLVQPGGSLRLSCAASGSTSTINRF |
| | | sequence of | AWVRQAPGKGLEWVSATSSRTSPNYADSVKGRFT |
| | | humanized | ISRDNSKNTLYLQMNSLRAEDTAVYYCAKRMAMG |
| | | VHH-2, | SSQVVYWGQGTTVTVSS |
| | | version 1 | |
| 110 | VHH-2.2 | Amino acid | EVQLVESGGGLVQPGGSLRLSCAVSGSTSTINRF |
| | | sequence of | AWYRQAPGKGLEWVAATSSRTSPNYADSVKGRFT |
| | | humanized | FSRDNSKNTVYLQMNSLRAEDTAVYYCQARMAMG |
| | | VHH-2, | SSQVVYWGQGTTVTVSS |
| | | version 2 | |
| 111 | VHH-2.3 | Amino acid | EVQLVESGGGLVQPGGSLRLSCAVSGSTSTINRF |
| | | sequence of | AWYRQAPGKGRELVAATSSRTSPNYADSVKGRFT |
| | | humanized | FSRDNSKNTVYLQMNSLRAEDTAVYYCQARMAMG |
| | | VHH-2, | SSQVVYWGQGTTVTVSS |
| | | version 3 | |

TABLE 9
Amino acid sequences of parental and
humanized framework regions corresponding
to humanized antibodies of Table 8.

| | Frame- | SEQ ID | |
|---|---|---|---|
| VHH | work | NO | Sequence |
| VHH-1 | FR1 | 163 | QVQLQESGGGLVQAGGSLRLSCVAS |
| | FR2 | 165 | IGWYRQAPGKQRELVAR |
| | FR3 | 169 | NYADFVKGRFTISGDNAKNTMYLQM |
| | | | NSLKPEDTAVYYC |
| | FR4 | 172 | WGQGTQVTVSS |
| VHH-1.1 | FR1 | 164 | EVQLVESGGGLVQPGGSLRLSCAAS |
| | FR2 | 166 | IGWVRQAPGKGLEWVSR |
| | FR3 | 170 | NYADSVKGRFTISRDNSKNTLYLQM |
| | | | NSLRAEDTAVYYC |
| | FR4 | 173 | WGQGTTVTVSS |
| VHH-1.2 | FR1 | 164 | EVQLVESGGGLVQPGGSLRLSCAAS |
| | FR2 | 167 | IGWYRQAPGKGLEWVAR |
| | FR3 | 171 | NYADSVKGRFTISGDNSKNTMYLQM |
| | | | NSLRAEDTAVYYC |
| | FR4 | 173 | WGQGTTVTVSS |
| VHH-1.3 | FR1 | 164 | EVQLVESGGGLVQPGGSLRLSCAAS |
| | FR2 | 168 | IGWYRQAPGKGRELVAR |
| | FR3 | 171 | NYADSVKGRFTISGDNSKNTMYLQM |
| | | | NSLRAEDTAVYYC |
| | FR4 | 173 | WGQGTTVTVSS |
| VHH-2 | FR1 | 175 | QVQLQESGGGLVQAGGSLRLTCAVS |
| | FR2 | 178 | FAWYRQAPGKQRELVAA |
| | FR3 | 182 | NYADSVKGRFRFSRDNARSAVYLQM |
| | | | DNLRPEDTAVYFC |
| | FR4 | 185 | WGLGTQVTVSS |
| VHH-2.1 | FR1 | 176 | EVQLVESGGGLVQPGGSLRLSCAAS |
| | FR2 | 179 | FAWVRQAPGKGLEWVSA |
| | FR3 | 183 | NYADSVKGRFTISRDNSKNTLYLQM |
| | | | NSLRAEDTAVYYC |
| | FR4 | 173 | WGQGTTVTVSS |
| VHH-2.2 | FR1 | 177 | EVQLVESGGGLVQPGGSLRLSCAVS |
| | FR2 | 180 | FAWYRQAPGKGLEWVAA |
| | FR3 | 184 | NYADSVKGRFTFSRDNSKNTVYLQM |
| | | | NSLRAEDTAVYYC |
| | FR4 | 173 | WGQGTTVTVSS |
| VHH-2.3 | FR1 | 177 | EVQLVESGGGLVQPGGSLRLSCAVS |
| | FR2 | 181 | FAWYRQAPGKGRELVAA |
| | FR3 | 184 | NYADSVKGRFTFSRDNSKNTVYLQM |
| | | | NSLRAEDTAVYYC |
| | FR4 | 173 | WGQGTTVTVSS |

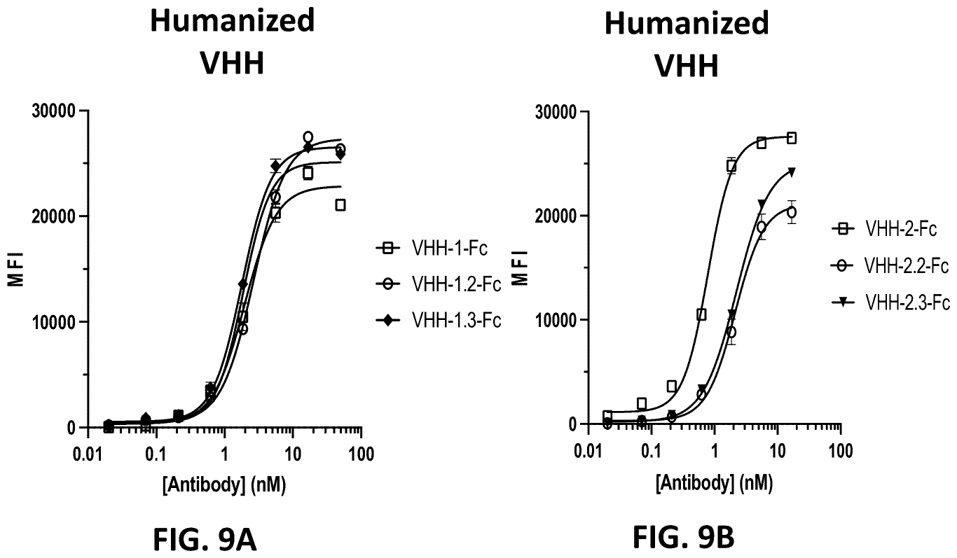

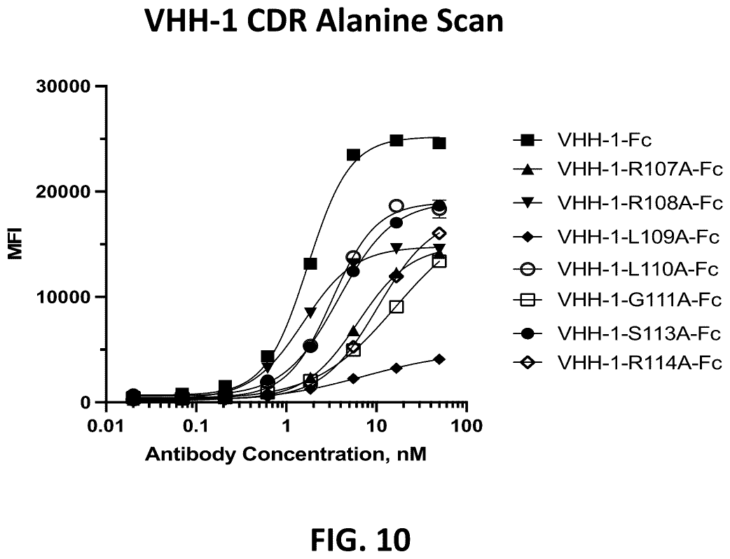

VHH-Fc fusion DNA constructs were then generated comprising encoding humanized VHH sequences (SEQ ID NOs: 106-111) fused in frame to the human IgG1 (SEQ ID NO: 34) directly to the short hinge sequence in the pFuse expression vector, and were expressed, purified and analyzed for binding to HEK293 cells expressing human CCR8 as in Example 5. Binding curves for VHH-Fc antibodies comprised of parental VHH-1 and the humanized VHH derivatives VHH-1.2-Fc and VHH-1.3-Fc are shown in FIG. 9A. Binding curves for VHH-Fc antibodies comprised of parental VHH-2 and the humanized VHH derivatives VHH-2-Fc, VHH-2.2-Fc and VHH-2.3-Fc, are shown in FIG. 9B. Cell binding EC50 values, normalized Bmax (relative to respective parental clone) data and humanizing mutations are shown in in Table 10 below.

TABLE 10
Binding Results for Parental and Humanized Antibodies

| Name | Humanizing Mutations (IMGT) | EC50 | Bmax (%) |
|---|---|---|---|
| VHH-1-Fc | None | 1.96 | 100.0 |
| VHH-1.2-Fc | Q1V, Q5V, A15P, V24A, Q49G, | 2.66 | 119.3 |
| | R50L, L52W, F70S, A83S, | | |
| | K95R, P96A, Q123T | | |
| VHH-1.3-Fc | Q1V, Q5V, A15P, V24A, Q49G, | 1.79 | 115.5 |
| | F70S, A83S, K95R, P96A, Q123T | | |
| VHH-2-Fc | None | 0.78 | 100.0 |
| VHH-2.2-Fc | Q1V, Q5V, A15P, Q49G, R50L, | 2.12 | 78.7 |
| | L52W, R77T, A83S, R84K, S85N, | | |
| | A86Y, L120Q, Q123T | | |
| VHH-2.3-Fc | Q1V, Q5V, A15P, Q49G, R77T, | 2.22 | 94.4 |
| | A83S, R84K, S85N, A86Y, | | |
| | L120Q, Q123T | | |

### Example 9. Humanized VHH Optimization by Affinity Maturation and Engineering

Humanization within the framework regions of VHH and other antibodies is known to often have negative impacts of on protein properties such as destabilization of the VHH fold or loss of binding affinity. This is most common when humanized versions of antibodies are constructed by “CDR grafting”, where the individual CDRs are transferred directly to a fully human framework. This was true of the most humanized version of VHH-1, VHH-1.1 (SEQ ID NO: 106) (17 of 19 possible humanizing mutations) and of VHH-2, VHH-2.1 (SEQ ID NO: 109) (20 of 22 possible humanizing mutations), which were destabilized to a point that they could not be produced and purified from cells. The less variants of the parental VHH-1 and VHH-2, VHH-1.2, (SEQ ID NO: 107) VHH.1.3, (SEQ ID NO: 108), VHH-2.2, (SEQ ID NO: 110), and VHH-2.3, (SEQ ID NO: 111) all maintained their hCCR8 binding properties as Fc fusion antibodies.

Approaches such as in vitro affinity maturation and rational mutagenesis can be performed to regain binding affinity and/or stability lost through humanization. In vitro affinity maturation generally involves building and amino acid substitution library where amino acid positions are varied across the CDRs and/or framework at a defined set of positions using sets of amino acids chosen to introduce new chemical diversity. The paratope residues within the CDR regions are of course key to antigen binding and care must be taken not to disrupt important structures while attempting to evolve improve antibody behavior. One approach to identify positions that are important to the maintenance of binding properties is to perform paratope mapping using single scanning alanine substitutions across antibody CDR regions. In this approach, residues that are critical to the maintenance of antibody binding to the antigen can be identified and at the same time permissive sites can be identified where it may be possible to make substitutions of other amino acids desirable for subsequent affinity maturation library construction. Site directed mutagenesis was used to make single alanine substitutions at each residue in the CDRs of VHH-1 (SEQ ID NO: 27) and VHH-2 (SEQ ID NO: 28). Changes in VHH-1 CDR amino acids to alanine are indicated in Table 11 (SEQ ID NOs: 112-137) and for VHH-2 CDRs (SEQ ID NO: 138-162) in Table 12. Mutation positions and residue changes to alanine with respect to the in the parental VHH sequences are referenced by their IMGT position. VHH domains containing the indicated modified CDR-1, CDR-2 or CDR-3 domains were used to construct Fc fusions by direct grafting to the short hinge of the human Fc domain (SEQ ID NO: 34). VHH-Fc constructs were expressed, purified and analyzed for binding to HEK293 cells expressing hCCR8 and the results are reported in Table 11 and Table 12. EC50 values are reported for each Fc fusion antibody containing a modified CDR, along with its respective Bmax value normalized to the to the matched wild type VHH-1-Fc or VHH-2-Fc fusion antibody.

TABLE 11
VHH-1 CDR Alanine Scanning
Sequences and Binding Data

| SEQ ID | | | Mutation | EC50 | % |
|---|---|---|---|---|---|
| NO | Sequence | Region | (IMGT) | (nM) | Bmax |
| 47 | GSTSSIRR | CDR1 | NONE | 1.73 | 100 |
| 112 | ASTSSIRR | CDR1 | G27A | 1.01 | 36 |
| 113 | GATSSIRR | CDR1 | S27A | 1.45 | 70 |
| 114 | GSASSIRR | CDR1 | T29A | 0.80 | 45 |
| 115 | GSTASIRR | CDR1 | S30A | 1.60 | 74 |
| 116 | GSTSAIRR | CDR1 | S35A | 1.26 | 69 |
| 117 | GSTSSARR | CDR1 | I36A | 1.22 | 35 |
| 118 | GSTSSIAR | CDR1 | R37A | 4.59 | 82 |
| 119 | GSTSSIRA | CDR1 | R38A | 2.47 | 69 |
| 48 | FSSGGGT | CDR2 | NONE | 1.73 | 100 |
| 120 | FASGGGT | CDR2 | S57A | 1.25 | 82 |
| 121 | FSAGGGT | CDR2 | S58A | 1.11 | 84 |
| 122 | FSSAGGT | CDR2 | G59A | 1.28 | 79 |
| 123 | FSSGAGT | CDR2 | G63A | 1.13 | 73 |
| 124 | FSSGGAT | CDR2 | G64A | 1.20 | 73 |
| 125 | FSSGGGA | CDR2 | T65A | 1.23 | 70 |
| 49 | SARRLLGTSRVDY | CDR3 | NONE | 1.73 | 100 |
| 126 | AARRLLGTSRVDY | CDR3 | S105A | 3.12 | 62 |
| 127 | SAARLLGTSRVDY | CDR3 | R107A | 7.65 | 48 |
| 128 | SARALLGTSRVDY | CDR3 | R108A | 1.59 | 48 |
| 129 | SARRALGTSRVDY | CDR3 | L109A | 5.97 | 15 |
| 130 | SARRLAGTSRVDY | CDR3 | L110A | 3.60 | 61 |
| 131 | SARRLLATSRVDY | CDR3 | G111A | 10.59 | 77 |
| 132 | SARRLLGASRVDY | CDR3 | T112A | 1.26 | 84 |
| 133 | SARRLLGTARVDY | CDR3 | S113A | 3.77 | 67 |
| 134 | SARRLLGTSAVDY | CDR3 | R114A | 16.43 | 78 |
| 135 | SARRLLGTSRADY | CDR3 | V115A | 7.19 | 77 |
| 136 | SARRLLGTSRVAY | CDR3 | D116A | 1.33 | 93 |
| 137 | SARRLLGTSRVDA | CDR3 | Y117A | 1.61 | 82 |

TABLE 12
VHH-2 CDR Alanine Scanning
Sequences and Data

| SEQ ID | | | Mutation | EC50 | % |
|---|---|---|---|---|---|
| NO | Sequence | Region | (IMGT) | (nM) | Bmax |
| 54 | GSTSTINR | CDR1 | NONE | 1.30 | 100 |
| 138 | ASTSTINR | CDR1 | G27A | 1.29 | 28 |
| 139 | GATSTINR | CDR1 | S28A | 1.61 | 68 |
| 140 | GSASTINR | CDR1 | T29A | 1.43 | 65 |
| 141 | GSTATINR | CDR1 | S30A | 1.62 | 66 |
| 142 | GSTSAINR | CDR1 | T35A | 0.87 | 65 |
| 143 | GSTSTANR | CDR1 | I36A | 0.84 | 50 |
| 144 | GSTSTIAR | CDR1 | N37A | 1.92 | 42 |
| 145 | GSTSTINA | CDR1 | R38A | 6.02 | 22 |
| 55 | TSSRTSP | CDR2 | NONE | 1.30 | 100 |
| 146 | TASRTSP | CDR2 | S57A | 1.03 | 69 |
| 147 | TSARTSP | CDR2 | S58A | 1.02 | 65 |
| 148 | TSSATSP | CDR2 | R59A | 2.47 | 68 |
| 149 | TSSRASP | CDR2 | T59A | 1.48 | 74 |
| 150 | TSSRTAP | CDR2 | S63A | 1.08 | 70 |
| 151 | TSSRTAA | CDR2 | P61A | 1.18 | 59 |
| 56 | QARMAMGSSQVVY | CDR3 | NONE | 1.30 | 100 |
| 152 | AARMAMGSSQVVY | CDR3 | Q105A | 1.90 | 73 |
| 153 | QAAMAMGSSQVVY | CDR3 | R107A | 5.98 | 21 |
| 154 | QARAAMGSSQVVY | CDR3 | M108A | 1.62 | 63 |
| 155 | QARMAAGSSQVVY | CDR3 | M110A | 1.92 | 58 |
| 156 | QARMAMASSQVVY | CDR3 | G111A | 1.43 | 33 |
| 157 | QARMAMGASQVVY | CDR3 | S112A | 0.89 | 88 |
| 158 | QARMAMGSAQVVY | CDR3 | S113A | 0.90 | 48 |
| 159 | QARMAMGSSAVVY | CDR3 | Q114A | 0.96 | 68 |
| 160 | QARMAMGSSQAVY | CDR3 | V115A | 1.34 | 11 |
| 161 | QARMAMGSSQVAY | CDR3 | V116A | 0.83 | 72 |
| 162 | QARMAMGSSQVVA | CDR3 | Y117A | 0.86 | 76 |

Alanine scanning mutagenesis data were used to identify sites in the CDR regions (CDR1, CDR2 and CDR3) of VHH-1 and VHH-2 that were permissive to substitutions and thus maintain sufficient target binding (Bmax>65% and EC50<5 nM) as determined by flow cytometry assay on cells expressing hCCR8 as described above. These data were used to guide the design of independent synthetic antibody variant libraries based on the sequences of VHH-1.3 and VHH-2.3 where amino acids variations were introduced at identified permissive sites within the CDRs and at several positions within CDR flanking framework regions, as they are important for CDR conformation and stability. The range of different amino acid substitutions at each of the chosen positions was determined from an analysis of natural antibody diversity using the AbYsis database (www.abysis.org). AbYsis was used to identify the most common amino acids in both camelid VHH and human VH domains at each site in the CDRs of VHH-1.3 (SEQ ID NOs: 47-49) and VHH-2.3 (SEQ ID NO: 54-56) and in CDR flanking framework regions over a window of several amino acids. Using the resulting diversity analysis data, antibody libraries were constructed using synthetic DNA technology (Twist Bioscience) containing variants with a range of substitutions, from one amino acid substitution per VHH coding sequence, to 7 substitutions mutations per VHH. The average number of mutations per VHH clone in the library was calculated to be 3.5. The theoretical diversity of the combinatorial library was 2×10⁷ clones, which is sufficiently low to allow good coverage and efficient selection after transformation in yeast.

Antibody libraries were cloned into a modified version of the yeast display plasmid pYD1, (NovoPro), designed for expression, secretion, and N-terminal display of proteins on the extracellular surface of Saccharomyces cerevisiae cells fused to the AGA2 gene. Fusion of a single domain antibody such as a scFv or VHH of the invention to the AGA2 N-terminus allows optimal secretion and display of the antibody on the surface of yeast. The AGA2 protein is linked by two disulfide bonds to the AGA1 protein linked covalently to the yeast cell wall.

VHH variant libraries were amplified by PCR with the addition of 50 bp vector-insert sequence overlaps and cloned into the linearized pYD1 vector by the Gap Repair method. DNA was transformed by electroporation in S. cerevisiae EBY100 using high efficiency competent cells. After library transformation the total calculated number of clones was equal to 2.5×10\hat{ }8. Yeast cells displaying the VHH variants on their surface were labeled with purified and fluorescently labeled hCCR8 antigen, which allowed detection of binding and sorting of yeast cells by FACS using a BIORAD S3E Cell sorter. Yeast were sorted in the first round of sorting using 140 nM, labeled hCCR8 antigen and then using 45 nM antigen for subsequent rounds. Plasmid DNA of bulk sorted yeast were isolated using and evaluated for enrichment of amino acid changes at specific CDRs positions by Next-Generation Sequencing (NGS) analysis. In addition, approximately 300 single sorted clones were isolated and tested for binding at 100 nM CCR8 antigen concentration. 64 clones were selected for further analysis with multiple concentrations of CCR8 antigen for binding on yeast by flow cytometry, which for determination of improved signal relative to yeast displaying the parental antibodies.

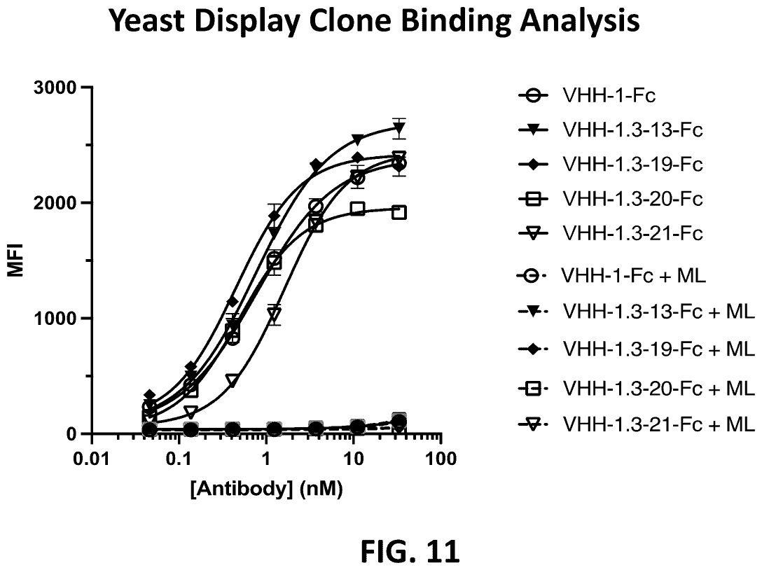

Top VHH sequences were chosen from the FACS sorted populations based on an analysis of improved binding performance relative to the parental sequences and evidence of sequence enrichment by NGS. Candidate VHH were then cloned as VHH-Fc fusions to the short hinge of the IgG1 Fc domain (SEQ ID NO: 34), expressed in HEK293 cells as described earlier and purified for binding evaluation. Binding of VHH-Fc antibodies to HuT-78 cells clones was analyzed by flow cytometry and the results are shown in Table 13. Bmax values are shown as normalized to VHH-1 or VHH-2 depending on the clone family. Mutations occurring in selected VHH clones are shown in the table and were found to occur both in the CDR and flanking framework regions. Mutations are referenced by position according to the IMGT definition. Representative binding curves for VHH-Fc fusion antibodies are shown are shown in FIG. 11.

TABLE 13
Binding evaluation of VHH Clones isolated by yeast display.

| SEQ | Clone | Mutations | EC50 | Normalized |
|---|---|---|---|---|
| ID NO | Name | (IMGT) | (nM) | Bmax (%) |
| 27 | VHH-1 | | 2.56 | 100.0 |
| 108 | VHH-1.3 | None (parent VHH) | 5.46 | 88.6 |
| 186 | VHH-1.3-1 | G64S | 2.03 | 85.3 |
| 187 | VHH-1.3-2 | L110V | 2.21 | 115.7 |
| 188 | VHH-1.3-3 | I39Y, G40S, R55W | 1.81 | 104.4 |
| 189 | VHH-1.3-4 | R38T, R108I | 7.68 | 82.4 |
| 190 | VHH-1.3-5 | R55G | 4.42 | 90.1 |
| 191 | VHH-1.3-6 | R55H | 3.75 | 97.0 |
| 192 | VHH-1.3-7 | A25V, R55H, N82K | 0.56 | 111.8 |
| 193 | VHH-1.3-8 | G63I, L110V | 1.36 | 63.4 |
| 194 | VHH-1.3-9 | S58G, G64S, T65I, L110V | 0.58 | 124.0 |
| 195 | VHH-1.3-10 | T65I, L110V | 1.30 | 125.4 |
| 196 | VHH-1.3-11 | G64T | 0.61 | 79.6 |
| 197 | VHH-1.3-12 | S28F, G64T, L110Y | 1.24 | 79.6 |
| 198 | VHH-1.3-13 | A25V, R38G, S57I | 0.38 | 91.1 |
| 199 | VHH-1.3-14 | G64S, L110V | 0.90 | 131.9 |
| 200 | VHH-1.3-15 | S58G | 2.82 | 107.1 |
| 201 | VHH-1.3-16 | S35T, R37V, G64S, R108I | 3.10 | 79.0 |
| 202 | VHH-1.3-17 | R37V, R55H, R108I, N83K | 0.67 | 72.8 |
| 203 | VHH-1.3-18 | A25V, R38G, S57I, N83K | 0.31 | 113.4 |
| 204 | VHH-1.3-19 | A25V, R38G, R55H, N83K | 0.45 | 130.0 |
| 205 | VHH-1.3-20 | A25V, R38G, S57I, R108I | 0.49 | 107.6 |
| 206 | VHH-1.3-21 | A25V, R38G, S57I, G81A | 1.62 | 133.9 |
| 28 | VHH-2 | None (parent VHH) | 0.24 | 100.0 |
| 111 | VHH-2.3 | Humanized Parent VHH | 1.19 | 88.8 |
| 207 | VHH-2.3-1 | D81G | 0.23 | 118.3 |
| 208 | VHH-2.3-2 | D81G, M108K | 0.12 | 131.9 |
| 209 | VHH-2.3-3 | P65L | 1.70 | 83.3 |

TABLE 14
Exemplary optimized humanized antibodies and corresponding groups of
complementarity determining regions and framework regions.
Sequences of corresponding full length VHH domains are provided Table 15

| VHH SEQ | | | | | | | | |
|---|---|---|---|---|---|---|---|---|
| ID NO | VHH Clone | CDR 1 | CDR 2 | CDR 3 | FR1 | FR2 | FR3 | FR4 |
| 186 | VHH-1.3-1 | 47 | 51 | 49 | 164 | 168 | 171 | 173 |
| 187 | VHH-1.3-2 | 47 | 48 | 227 | 164 | 168 | 171 | 173 |
| 188 | VHH-1.3-3 | 47 | 48 | 49 | 164 | 231 | 171 | 173 |
| 189 | VHH-1.3-4 | 210 | 48 | 226 | 164 | 168 | 171 | 173 |
| 190 | VHH-1.3-5 | 47 | 48 | 49 | 164 | 232 | 171 | 173 |
| 191 | VHH-1.3-6 | 47 | 48 | 49 | 164 | 233 | 171 | 173 |
| 192 | VHH-1.3-7 | 47 | 48 | 49 | 177 | 233 | 234 | 173 |
| 193 | VHH-1.3-8 | 47 | 216 | 227 | 164 | 168 | 171 | 173 |
| 194 | VHH-1.3-9 | 47 | 217 | 227 | 164 | 168 | 171 | 173 |
| 195 | VHH-1.3-10 | 47 | 218 | 227 | 164 | 168 | 171 | 173 |
| 196 | VHH-1.3-11 | 47 | 219 | 49 | 164 | 168 | 171 | 173 |
| 197 | VHH-1.3-12 | 211 | 220 | 228 | 164 | 168 | 171 | 173 |
| 198 | VHH-1.3-13 | 212 | 221 | 49 | 177 | 168 | 171 | 173 |
| 199 | VHH-1.3-14 | 47 | 222 | 227 | 164 | 168 | 171 | 173 |
| 200 | VHH-1.3-15 | 47 | 223 | 49 | 164 | 168 | 171 | 173 |
| 201 | VHH-1.3-16 | 213 | 51 | 226 | 164 | 168 | 171 | 173 |
| 202 | VHH-1.3-17 | 214 | 48 | 226 | 177 | 233 | 234 | 173 |
| 203 | VHH-1.3-18 | 212 | 221 | 49 | 177 | 168 | 234 | 173 |
| 204 | VHH-1.3-19 | 212 | 48 | 49 | 177 | 233 | 234 | 173 |
| 205 | VHH-1.3-20 | 212 | 221 | 226 | 177 | 168 | 171 | 173 |
| 206 | VHH-1.3-21 | 212 | 221 | 49 | 177 | 168 | 235 | 173 |
| 207 | VHH-2.3-1 | 54 | 55 | 56 | 177 | 181 | 236 | 173 |
| 208 | VHH-2.3-2 | 54 | 55 | 229 | 177 | 181 | 236 | 173 |
| 209 | VHH-2.3-3 | 54 | 224 | 56 | 177 | 181 | 184 | 173 |

TABLE 15
Amino acid sequences of exemplary optimized humanized
VHH-Fc antibodies of the disclosure

| SEQ ID | | |
|---|---|---|
| NO | Clone Name | VHH Sequence |
| 186 | VHH-1.3-1 | EVQLVESGGGLVQPGGSLRLSCAASGSTSSIRRIGWYRQAPGKGRE |
| | | LVARFSSGGSTNYADSVKGRFTISGDNSKNTMYLQMNSLRAEDTAV |
| | | YYCSARRLLGTSRVDYWGQGTTVTVSS |
| 187 | VHH-1.3-2 | EVQLVESGGGLVQPGGSLRLSCAASGSTSSIRRIGWYRQAPGKGRE |
| | | LVARFSSGGGTNYADSVKGRFTISGDNSKNTMYLQMNSLRAEDTAV |
| | | YYCSARRLVGTSRVDYWGQGTTVTVSS |
| 188 | VHH-1.3-3 | EVQLVESGGGLVQPGGSLRLSCAASGSTSSIRRYSWYRQAPGKGRE |
| | | LVAWFSSGGGTNYADSVKGRFTISGDNSKNTMYLQMNSLRAEDTAV |
| | | YYCSARRLLGTSRVDYWGQGTQVTVSS |
| 189 | VHH-1.3-4 | EVQLVESGGGLVQPGGSLRLSCAASGSTSSIRTIGWYRQAPGKGRE |
| | | LVARFSSGGGTNYADSVKGRFTISGDNSKNTMYLQMNSLRAEDTAV |
| | | YYCSARILLGTSRVDYWGQGTTVTVSS |
| 190 | VHH-1.3-5 | EVQLVESGGGLVQPGGSLRLSCAASGSTSSIRRIGWYRQAPGKGRE |
| | | LVAGFSSGGGTNYADSVKGRFTISGDNSKNTMYLQMNSLRAEDTAV |
| | | YYCSARRLLGTSRVDYWGQGTTVTVSS |
| 191 | VHH-1.3-6 | EVQLVESGGGLVQPGGSLRLSCAASGSTSSIRRIGWYRQAPGKGRE |
| | | LVAHFSSGGGTNYADSVKGRFTISGDNSKNTMYLQMNSLRAEDTAV |
| | | YYCSARRLLGTSRVDYWGQGTTVTVSS |
| 192 | VHH-1.3-7 | EVQLVESGGGLVQPGGSLRLSCAVSGSTSSIRRIGWYRQAPGKGRE |
| | | LVAHFSSGGGTNYADSVKGRFTISGDKSKNTMYLQMNSLRAEDTAV |
| | | YYCSARRLLGTSRVDYWGQGTTVTVSS |
| 193 | VHH-1.3-8 | EVQLVESGGGLVQPGGSLRLSCAASGSTSSIRRIGWYRQAPGKGRE |
| | | LVARFSSGIGTNYADSVKGRFTISGDNSKNTMYLQMNSLRAEDTAV |
| | | YYCSARRLVGTSRVDYWGQGTQVTVSS |
| 194 | VHH-1.3-9 | EVQLVESGGGLVQPGGSLRLSCAASGSTSSIRRIGWYRQAPGKGRE |
| | | LVARFSGGGSINYADSVKGRFTISGDNSKNTMYLQMNSLRAEDTAV |
| | | YYCSARRLVGTSRVDYWGQGTQVTVSS |
| 195 | VHH-1.3-10 | QVQLQESGGGLVQPGGSLRLSCAASGSTSSIRRIGWYRQAPGKGRE |
| | | LVARFSSGGGINYADSVKGRFTISGDNSKNTMYLQMNSLRAEDTAV |
| | | YYCSARRLVGTSRVDYWGQGTQVTVSS |
| 196 | VHH-1.3-11 | QVQLQESGGGLVQPGGSLRLSCAASGSTSSIRRIGWYRQAPGKGRE |
| | | LVARFSSGGTINYADSVKGRFTISGDNSKNTMYLQMNSLRAEDTAV |
| | | YYCSARRLLGTSRVDYWGQGTQVTVSS |
| 197 | VHH-1.3-12 | EVQLVESGGGLVQPGGSLRLSCAASGFTSSIRRIGWYRQAPGKGRE |
| | | LVARFSSGGTTNYADSVKGRFTISGDNSKNTMYLQMNSLRAEDTAV |
| | | YYCSARRLYGTSRVDYWGQGTQVTVSS |
| 198 | VHH-1.3-13 | EVQLVESGGGLVQPGGSLRLSCAVSGSTSSIRGIGWYRQAPGKGRE |
| | | LVARFISGGGTNYADSVKGRFTISGDNSKNTMYLQMNSLRAEDTAV |
| | | YYCSARRLLGTSRVDYWGQGTQVTVSS |
| 199 | VHH-1.3-14 | EVQLVESGGGLVQPGGSLRLSCAASGSTSSIRRIGWYRQAPGKGRE |
| | | LVARFSSGGSINYADSVKGRFTISGDNSKNTMYLQMNSLRAEDTAV |
| | | YYCSARRLVGTSRVDYWGQGTQVTVSS |
| 200 | VHH-1.3-15 | EVQLVESGGGLVQPGGSLRLSCAASGSTSSIRRIGWYRQAPGKGRE |
| | | LVARFSGGGGTNYADSVKGRFTISGDNSKNTMYLQMNSLRAEDTAV |
| | | YYCSARRLLGTSRVDYWGQGTTVTVSS |
| 201 | VHH-1.3-16 | EVQLVESGGGLVQPGGSLRLSCAASGSTSTIVRIGWYRQAPGKGRE |
| | | LVARFSSGGSTNYADSVKGRFTISGDNSKNTMYLQMNSLRAEDTAV |
| | | YYCSARILLGTSRVDYWGQGTTVTVSS |
| 202 | VHH-1.3-17 | EVQLVESGGGLVQPGGSLRLSCAVSGSTSSIVRIGWYRQAPGKGRE |
| | | LVAHFSSGGGTNYADSVKGRFTISGDKSKNTMYLQMNSLRAEDTAV |
| | | YYCSARILLGTSRVDYWGQGTTVTVSS |
| 203 | VHH-1.3-18 | EVQLVESGGGLVQPGGSLRLSCAVSGSTSSIRGIGWYRQAPGKGRE |
| | | LVARFISGGGTNYADSVKGRFTISGDKSKNTMYLQMNSLRAEDTAV |
| | | YYCSARRLLGTSRVDYWGQGTTVTVSS |
| 204 | VHH-1.3-19 | EVQLVESGGGLVQPGGSLRLSCAVSGSTSSIRGIGWYRQAPGKGRE |
| | | LVAHFSSGGGTNYADSVKGRFTISGDKSKNTMYLQMNSLRAEDTAV |
| | | YYCSARRLLGTSRVDYWGQGTQVTVSS |
| 205 | VHH-1.3-20 | EVQLVESGGGLVQPGGSLRLSCAVSGSTSSIRGIGWYRQAPGKGRE |
| | | LVARFISGGGTNYADSVKGRFTISGDNSKNTMYLQMNSLRAEDTAV |
| | | YYCSARILLGTSRVDYWGQGTQVTVSS |
| 206 | VHH-1.3-21 | EVQLVESGGGLVQPGGSLRLSCAVSGSTSSIRGIGWYRQAPGKGRE |
| | | LVARFISGGGTNYADSVKGRFTISADNSKNTMYLQMNSLRAEDTAV |
| | | YYCSARRLLGTSRVDYWGQGTQVTVSS |
| 207 | VHH-2.3-1 | EVQLVESGGGLVQPGGSLRLSCAVSGSTSTINRFAWYRQAPGKGRE |
| | | LVAATSSRTSPNYADSVKGRFTFSRGNSKNTVYLQMNSLRAEDTAV |
| | | YYCQARMAMGSSQVVYWGQGTTVTVSS |
| 208 | VHH-2.3-2 | EVQLVESGGGLVQPGGSLRLSCAVSGSTSTINRFAWYRQAPGKGRE |
| | | LVAATSSRTSPNYADSVKGRFTFSRGNSKNTVYLQMNSLRAEDTAV |
| | | YYCQARKAMGSSQVVYWGQGTTVTVSS |
| 209 | VHH-2.3-3 | EVQLVESGGGLVQPGGSLRLSCAVSGSTSTINRFAWYRQAPGKGRE |
| | | LVAATSSRTSLNYADSVKGRFTFSRDNSKNTVYLQMNSLRAEDTAV |
| | | YYCQARMAMGSSQVVYWGQGTTVTVSS |

TABLE 16
Exemplary amino acid sequences of
complementarity determining regions
(CDR) and framework regions (FR)
corresponding to the optimized
humanized VHH antibodies of Table 15.

| SEQ | | |
|---|---|---|
| ID | Descrip- | |
| NO | tion | Sequence |
| 210 | CDR1 | GSTSSIRT |
| 211 | CDR1 | GFTSSIRR |
| 212 | CDR1 | GSTSSIRG |
| 213 | CDR1 | GSTSTIVR |
| 214 | CDR1 | GSTSSIVR |
| 216 | CDR2 | FSSGIGT |
| 217 | CDR2 | FSGGGSI |
| 218 | CDR2 | FSSGGGI |
| 219 | CDR2 | FSSGGTI |
| 220 | CDR2 | FSSGGTT |
| 221 | CDR2 | FISGGGT |
| 222 | CDR2 | FSSGGSI |
| 223 | CDR2 | FSGGGGT |
| 224 | CDR2 | TSSRTSL |
| 226 | CDR3 | SARILLGTSRVDY |
| 227 | CDR3 | SARRLVGTSRVDY |
| 228 | CDR3 | SARRLYGTSRVDY |
| 229 | CDR3 | QARKAMGSSQVVY |
| 232 | FR2 | IGWYRQAPGKGRELVAG |
| 233 | FR2 | IGWYRQAPGKGRELVAH |
| 234 | FR3 | NYADSVKGRFTISGDKSKNTMYLQMNSLRAEDTAV |
| | | YYC |
| 235 | FR3 | NYADSVKGRFTISADNSKNTMYLQMNSLRAEDTAV |
| | | YYC |
| 236 | FR3 | NYADSVKGRFTFSRGNSKNTVYLQMNSLRAEDTAV |
| | | YYC |
| 238 | Mouse | EPRGPTIKPCPPCKCPAPNLLGGPSVFIFPPKIKD |
| | IgG2a | VLMISLSPIVTCVVVDVSEDDPDVQISWFVNNVEV |
| | Fc | HTAQTQTHREDYNSTLRVVSALPIQHQDWMSGKEF |
| | domain | KCKVNNKDLPAPIERTISKPKGSVRAPQVYVLPPP |
| | and | EEEMTKKQVTLTCMVTDFMPEDIYVEWTNNGKTEL |
| | hinge | NYKNTEPVLDSDGSYFMYSKLRVEKKNWVERNSYS |
| | | CSVVHEGLHNHHTTKSFSRTPGK |
| 239 | VHH-1- | QVQLQESGGGLVQAGGSLRLSCVASGSTSSIRRIG |
| | mFc | WYRQAPGKQRELVARFSSGGGTNYADFVKGRFTIS |
| | | GDNAKNTMYLQMNSLKPEDTAVYYCSARRLLGTSR |
| | | VDYWGQGTQVTVSSEPRGPTIKPCPPCKCPAPNLL |
| | | GGPSVFIFPPKIKDVLMISLSPIVTCVVVDVSEDD |
| | | PDVQISWFVNNVEVHTAQTQTHREDYNSTLRVVSA |
| | | LPIQHQDWMSGKEFKCKVNNKDLPAPIERTISKPK |
| | | GSVRAPQVYVLPPPEEEMTKKQVTLTCMVTDFMPE |
| | | DIYVEWTNNGKTELNYKNTEPVLDSDGSYFMYSKL |
| | | RVEKKNWVERNSYSCSVVHEGLHNHHTTKSFSRTP |
| | | GK |
| 240 | BMS-4A19- | QVQLVQSGAEVKKPGASVKVSCKASGYTFTDSEMH |
| | DE | WVRQATGQGLEWMGAIQPETGGTAYNQKFKARVTM |
| | (Contains | TRDTSISTAYMELSSLRSEDTAVYYCARRRRNFDY |
| | DE Fc | WGQGTLVTVSSASTKGPSVFPLAPSSKSTSGGTAA |
| | effector | LGCLVKDYFPEPVTVSWNSGALTSGVHTFPAVLQS |
| | enhancing | SGLYSLSSVVTVPSSSLGTQTYICNVNHKPSNTKD |
| | mutations) | KRVEPKSCDKTHTCPPCPAPELLGGPDVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALPAPEEKTISKAKGQPREPQVYTLP |
| | | PSREEMTKNQVSLTCLVKGFYPSDIAVEWESNGQP |
| | | ENNYKTTPPVLDSDGSFFLYSKLTVDKSRWQQGNV |
| | | FSCSVMHEALHNHYTQKSLSLSPG |
| 243 | CDR3 | SARRLLGLSRVDY |
| 244 | CDR3 | SARRLLGTSRVNY |
| 245 | CDR3 | SARRLLGTSRVGY |
| 246 | CDR3 | SARRLLGTSRVDS |

### Example 10. Functional Evaluation of Optimized Lead VHH and VHH-Fc Molecules

VHH-Fc fusions were created by direct fusion to the to the short hinge of the human IgG1 Fc (SEQ ID NO: 34) as described previously, or alternatively, as direct fusions of the individual VHH domains to the hinge region of the mouse IgG2a, (SEQ ID NO: 238), referred to as “VHH-mFc” fusions. One particular example of this antibody format is VHH-1-mFc (SEQ ID NO: 239), where the VHH portion of the Fc fusion molecule can be readily identified by its name as VHH-1 (SEQ ID NO: 27).

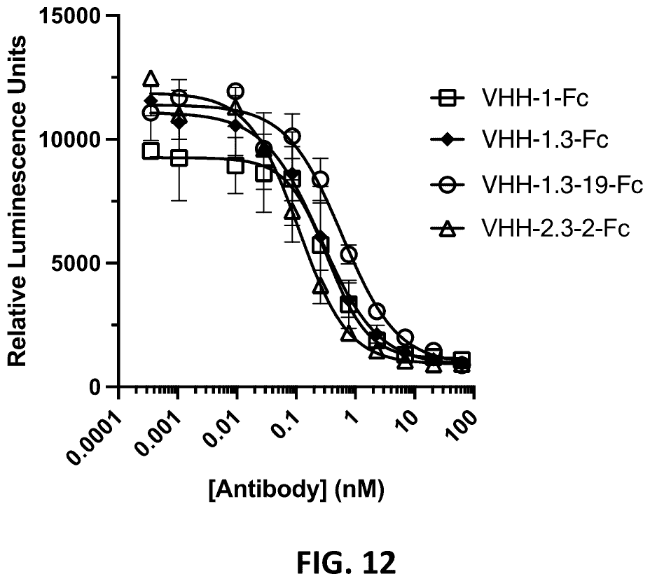

Selected optimized VHH-Fc molecules were evaluated for functional inhibition of CCL1-induced hCCR8 activation and recruitment of β-arrestin (DiscoverX PathHunter® eXpress, Eurofins) signaling (FIG. 12). In this assay, a fixed concentration (EC80) of the hCCR8 agonist CCL1 (R&D Systems, #272-I) was used to stimulate receptor b-arrestin signaling. Multipoint dilution series of individual VHH-Fc molecules were mixed with the assay components to generate inhibition curves, from which the concentration necessary to achieve 50% inhibition (IC50) could be determined. Data were plotted and fit using GraphPad Prism software and IC50 values individual antibodies were calculated from the resulting dose-response curves. Fc fusions of VHH-1 (SEQ ID NO: 27), VHH-1.3 (SEQ ID NO: 108), VHH-1.3-19 (SEQ ID NO: 204), and VHH-2.3-2 (SEQ ID NO: 208) were evaluated and found to be potent inhibitors as shown by the sub-nanomolar potency values listed in in Table 17.

TABLE 17
Inhibition potencies of VHH-Fc fusions in β-arrestin assay

| VHH SEQ ID NO | Fc Fusion Antibody | IC50 (nM) |
|---|---|---|
| 27 | VHH-1-Fc | 0.34 |
| 108 | VHH-1.3-Fc | 0.26 |
| 204 | VHH-1.3-19-Fc | 0.59 |
| 208 | VHH-2.3-2-Fc | 0.11 |

### Example 11. Evaluation of Optimized Lead VHH-Fc or ADCC Effector Activity

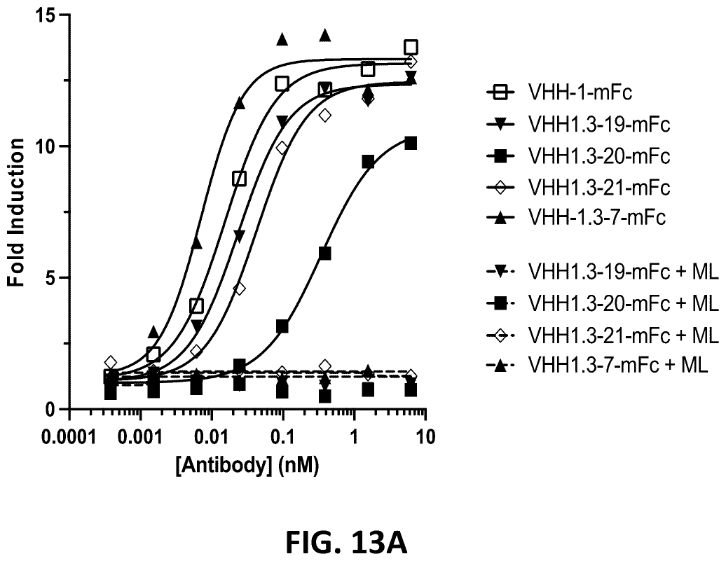

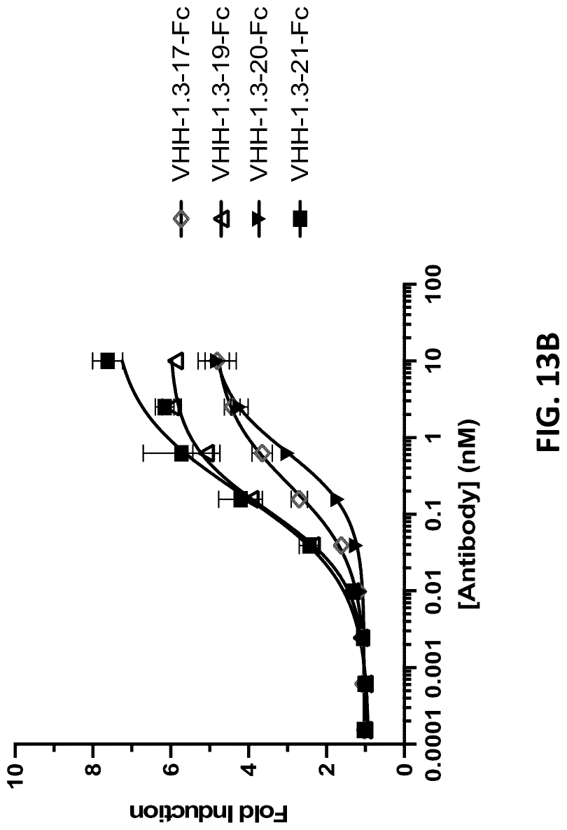

VHH-Fc fusions in the human and mouse Fc fusion format were generated as described above and a set were tested for their capacity to activate either the human FcgRIIIa or the mouse FcgRIV in the ADCC reporter assay (Promega, G7010, G7018). In this assay engineered Jurkat cells are used as effector cells that are stably transfected with the V158 FcγRIIIa receptor and an NFAT (nuclear factor of activated T-cells) responsive firefly luciferase reporter gene. HuT-78 cells that naturally express human CCR8 were used as target cells. ADCC activity was quantified through the produced luciferase luminescence signal resulting from the NFAT pathway activation upon incubation of the VHH-Fc fusions with the target and effector cells at a 2.5:1 effector:target cell ratio, according to the recommendations of the manufacturer. This assay design was used to determine the effector function of human IgG1 chimera and humanized versions of anti-CCR8. Briefly, Hut-78 cells were resuspended in prewarmed assay buffer (37° C.) at 106 cells/mL concentration. 25,000 cells were mixed with serially diluted anti-CCR8 antibodies in 96-well flat, clear-bottom plate, and then incubated for 1 hour at 37° C., 5% CO2. Promega BioAssay effector cells were added to individual well at varied ratios to target cells and further incubated for 6 hours at 37° C. 5% CO2. After incubation, assay plates were equilibrated to room temperature for 15 min under foil on the benchtop. Pre-mixed Bio-Glo Luciferase Assay Substrate was added to each well and incubated at room temperature for 5 min. The assay plates were read on a Tecan plate reader within 30 min of substrate addition. Data was plotted using GraphPad Prism software to generate EC50 and fold induction values for individual antibodies. Fold induction is defined as the average maximum signal achieved in the assay (in RLU) divided by the average background signal with no antibody added. Results for these studies are shown in FIG. 13A and FIG. 13B. Antibodies tested in the assays and results of ADCC assays are shown in Table 18.

TABLE 18
ADCC assay activity of VHH-Fc and VHH-mFc fusions

| VHH SEQ | Fc Fusion | EC50 | Fold |
|---|---|---|---|
| ID NO | Antibody | (nM) | Induction |
| 27 | VHH-1-mFc | 0.015 | 12.01 |
| 192 | VHH-1.3-7-mFc | 0.007 | 12.04 |
| 204 | VHH1.3-19-mFc | 0.024 | 11.31 |
| 205 | VHH1.3-20-mFc | 0.345 | 9.736 |
| 206 | VHH1.3-21-mFc | 0.042 | 11.37 |
| 202 | VHH-1.3-17-Fc | 0.24 | 3.92 |
| 204 | VHH-1.3-19-Fc | 0.11 | 5.06 |
| 205 | VHH-1.3-20-Fc | 0.65 | 3.98 |
| 206 | VHH-1.3-21-Fc | 0.19 | 6.68 |

### Example 12. Tumor Growth Inhibition Study

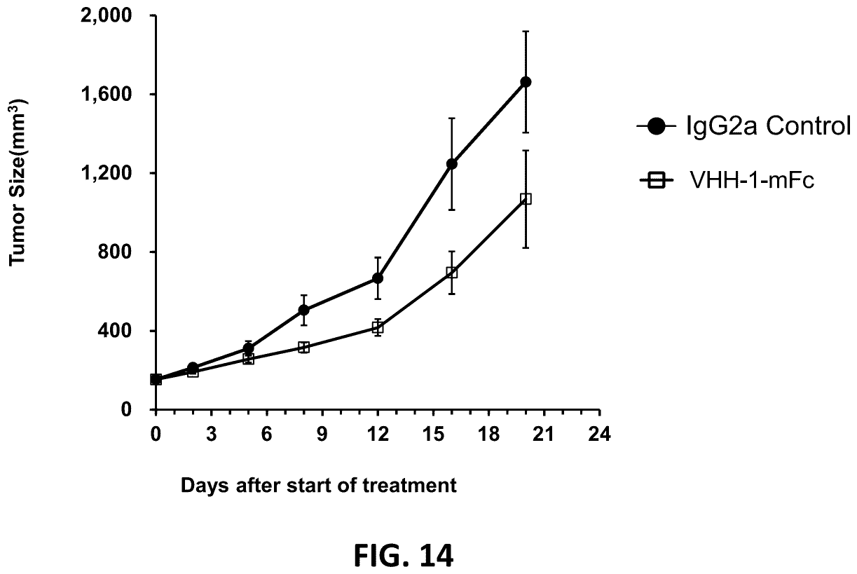

Human CCR8 transgenic (hCCR8-tg) mice (#110096, Biocytogen, China) were inoculated with MC38 mouse colon cancer cells subcutaneously. After the average tumor size reached approximately 150 mm3, mice were randomized based on tumor size and treatment was initiated. Mice were treated IV 3 times per week with 30 mg/kg VHH-1-mFc (SEQ ID NO: 239) or an IgG2a Fc control (recombinant IgG2a Fc, #BE0097, BioXcell). Tumor volume was measured twice per week using a caliper, and the volume expressed in mm3 using the formula: V (mm3)=(a×b2)/2 where a and b were the long and short diameters of the tumor, respectively. The tumor size was then used for calculations of tumor growth inhibition (T/C) values (where T was the treatment group and C is the control group). The T/C value (in percent) is an indication of antitumor effectiveness; T and C were the mean volume of the treated and control groups, respectively, on a given day. The average tumor volume at Day 16 (the last time point with all animals remaining in the study) in the hCCR8 Tg mice from VHH-1-mFc (30 mpk) were significantly smaller than the average tumor volume of the control G1-Isotype control (10 mpk). To determine whether group treated with various test articles had statistically significant anti-tumor effects compared to the mouse IgG2a Fc control, the MC38 tumor volumes were analyzed by one-way ANOVA and multiple comparison test at day 16. Results are shown in FIG. 14. The test article VHH-1-mFc displayed significantly higher anti-tumor efficacy than the isotype control and had a 1.8-fold smaller tumor volume (p=0.0169). In addition, the tumor growth inhibition (TGI) was also calculated during the study until Day 16. On Day 16, the TGIs for VHH-1-mFc were 50.3%, (p=0.0169).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-128. (canceled)

129. An isolated antibody that comprises:
a Chemokine receptor 8 (CCR8) binding domain that comprises a CDR1 region, a CDR2 region, and a CDR3 region,
wherein the CDR1 region comprises an amino acid sequence with at least 80% sequence identity to any one of SEQ ID NOs: 2, 3, 11, 12, 47, 54, 57, 58, 112-119, 139-145, or 210-214,
wherein the CDR2 region comprises an amino acid sequence with at least 80% sequence identity to any one of SEQ ID NOs: 4, 5, 6, 13, 14, 15, 48, 50, 51, 55, 59, 60, 120-125, 146-151, or 216-224, and
wherein the CDR3 region comprises an amino acid sequence with at least 80% sequence identity to any one of SEQ ID NOs: 8, 9, 17, 49, 52, 53, 56, 61, 62, 63, 64, 65, 66, 67, 68, 69, 126-137, 152-162, 225-229, 243-246, wherein the sequence identity is determined by Blastp.

130. The isolated antibody of claim 129, wherein
(a) the CDR1 region comprises an amino acid sequence of SEQ ID NO: 47, the CDR2 region comprises an amino acid sequence of SEQ ID NO: 48, and wherein the CDR3 region comprises an amino acid sequence of SEQ ID NO: 49,
(b) the CDR1 region comprises an amino acid sequence of SEQ ID NO: 54, the CDR2 region comprises an amino acid sequence of SEQ ID NO: 13, and wherein the CDR3 region comprises an amino acid sequence of SEQ ID NO: 56,
(c) the CDR1 region comprises an amino acid sequence of SEQ ID NO: 47, the CDR2 region comprises an amino acid sequence of SEQ ID NO: 6, and wherein the CDR3 region comprises an amino acid sequence of SEQ ID NO: 8,
(d) the CDR1 region comprises an amino acid sequence of SEQ ID NO: 54, the CDR2 region comprises an amino acid sequence of SEQ ID NO: 13, and wherein the CDR3 region comprises an amino acid sequence of SEQ ID NO: 157,
(e) the CDR1 region comprises an amino acid sequence of SEQ ID NO: 47, the CDR2 region comprises an amino acid sequence of SEQ ID NO: 48, and wherein the CDR3 region comprises an amino acid sequence of SEQ ID NO: 49,
(f) the CDR1 region comprises an amino acid sequence of SEQ ID NO: 47, the CDR2 region comprises an amino acid sequence of SEQ ID NO: 48, and wherein the CDR3 region comprises an amino acid sequence of SEQ ID NO: 52,
(g) the CDR1 region comprises an amino acid sequence of SEQ ID NO: 47, the CDR2 region comprises an amino acid sequence of SEQ ID NO: 48, and wherein the CDR3 region comprises an amino acid sequence of SEQ ID NO: 53,
(h) the CDR1 region comprises an amino acid sequence of SEQ ID NO: 47, the CDR2 region comprises an amino acid sequence of SEQ ID NO: 50, and wherein the CDR3 region comprises an amino acid sequence of SEQ ID NO: 52,
(i) the CDR1 region comprises an amino acid sequence of SEQ ID NO: 47, the CDR2 region comprises an amino acid sequence of SEQ ID NO: 51, and wherein the CDR3 region comprises an amino acid sequence of SEQ ID NO: 243,
(j) the CDR1 region comprises an amino acid sequence of SEQ ID NO: 47, the CDR2 region comprises an amino acid sequence of SEQ ID NO: 50, and wherein the CDR3 region comprises an amino acid sequence of SEQ ID NO: 53,
(k) the CDR1 region comprises an amino acid sequence of SEQ ID NO: 47, the CDR2 region comprises an amino acid sequence of SEQ ID NO: 51, and wherein the CDR3 region comprises an amino acid sequence of SEQ ID NO: 244,
(l) the CDR1 region comprises an amino acid sequence of SEQ ID NO: 47, the CDR2 region comprises an amino acid sequence of SEQ ID NO: 50, and wherein the CDR3 region comprises an amino acid sequence of SEQ ID NO: 245,
(m) the CDR1 region comprises an amino acid sequence of SEQ ID NO: 47, the CDR2 region comprises an amino acid sequence of SEQ ID NO: 50, and wherein the CDR3 region comprises an amino acid sequence of SEQ ID NO: 246,
(n) the CDR1 region comprises an amino acid sequence of SEQ ID NO: 54, the CDR2 region comprises an amino acid sequence of SEQ ID NO: 55, and wherein the CDR3 region comprises an amino acid sequence of SEQ ID NO: 56,
(o) the CDR1 region comprises an amino acid sequence of SEQ ID NO: 54, the CDR2 region comprises an amino acid sequence of SEQ ID NO: 55, and wherein the CDR3 region comprises an amino acid sequence of SEQ ID NO: 61,
(p) the CDR1 region comprises an amino acid sequence of SEQ ID NO: 54, the CDR2 region comprises an amino acid sequence of SEQ ID NO: 59, and wherein the CDR3 region comprises an amino acid sequence of SEQ ID NO: 62,
(q) the CDR1 region comprises an amino acid sequence of SEQ ID NO: 54, the CDR2 region comprises an amino acid sequence of SEQ ID NO: 60, and wherein the CDR3 region comprises an amino acid sequence of SEQ ID NO: 63,
(r) the CDR1 region comprises an amino acid sequence of SEQ ID NO: 54, the CDR2 region comprises an amino acid sequence of SEQ ID NO: 60, and wherein the CDR3 region comprises an amino acid sequence of SEQ ID NO: 64,
(s) the CDR1 region comprises an amino acid sequence of SEQ ID NO: 54, the CDR2 region comprises an amino acid sequence of SEQ ID NO: 55, and wherein the CDR3 region comprises an amino acid sequence of SEQ ID NO: 65,
(t) the CDR1 region comprises an amino acid sequence of SEQ ID NO: 57, the CDR2 region comprises an amino acid sequence of SEQ ID NO: 55, and wherein the CDR3 region comprises an amino acid sequence of SEQ ID NO: 66,
(u) the CDR1 region comprises an amino acid sequence of SEQ ID NO: 58, the CDR2 region comprises an amino acid sequence of SEQ ID NO: 55, and wherein the CDR3 region comprises an amino acid sequence of SEQ ID NO: 67,
(v) the CDR1 region comprises an amino acid sequence of SEQ ID NO: 54, the CDR2 region comprises an amino acid sequence of SEQ ID NO: 55, and wherein the CDR3 region comprises an amino acid sequence of SEQ ID NO: 68,
(w) the CDR1 region comprises an amino acid sequence of SEQ ID NO: 54, the CDR2 region comprises an amino acid sequence of SEQ ID NO: 60, and wherein the CDR3 region comprises an amino acid sequence of SEQ ID NO: 69,
(x) the CDR1 region comprises an amino acid sequence of SEQ ID NO: 47, the CDR2 region comprises an amino acid sequence of SEQ ID NO: 51, and wherein the CDR3 region comprises an amino acid sequence of SEQ ID NO: 49,
(y) the CDR1 region comprises an amino acid sequence of SEQ ID NO: 47, the CDR2 region comprises an amino acid sequence of SEQ ID NO: 48, and wherein the CDR3 region comprises an amino acid sequence of SEQ ID NO: 227,
(z) the CDR1 region comprises an amino acid sequence of SEQ ID NO: 47, the CDR2 region comprises an amino acid sequence of SEQ ID NO: 48, and wherein the CDR3 region comprises an amino acid sequence of SEQ ID NO: 49,
(aa) the CDR1 region comprises an amino acid sequence of SEQ ID NO: 210, the CDR2 region comprises an amino acid sequence of SEQ ID NO: 48, and wherein the CDR3 region comprises an amino acid sequence of SEQ ID NO: 226,
(bb) the CDR1 region comprises an amino acid sequence of SEQ ID NO: 47, the CDR2 region comprises an amino acid sequence of SEQ ID NO: 216, and wherein the CDR3 region comprises an amino acid sequence of SEQ ID NO: 227,
(cc) the CDR1 region comprises an amino acid sequence of SEQ ID NO: 47, the CDR2 region comprises an amino acid sequence of SEQ ID NO: 217, and wherein the CDR3 region comprises an amino acid sequence of SEQ ID NO: 227,
(dd) the CDR1 region comprises an amino acid sequence of SEQ ID NO: 47, the CDR2 region comprises an amino acid sequence of SEQ ID NO: 218, and wherein the CDR3 region comprises an amino acid sequence of SEQ ID NO: 227,
(ee) the CDR1 region comprises an amino acid sequence of SEQ ID NO: 47, the CDR2 region comprises an amino acid sequence of SEQ ID NO: 219, and wherein the CDR3 region comprises an amino acid sequence of SEQ ID NO: 49,
(ff) the CDR1 region comprises an amino acid sequence of SEQ ID NO: 211, the CDR2 region comprises an amino acid sequence of SEQ ID NO: 220, and wherein the CDR3 region comprises an amino acid sequence of SEQ ID NO: 228,
(gg) the CDR1 region comprises an amino acid sequence of SEQ ID NO: 212, the CDR2 region comprises an amino acid sequence of SEQ ID NO: 221, and wherein the CDR3 region comprises an amino acid sequence of SEQ ID NO: 49,
(hh) the CDR1 region comprises an amino acid of SEQ ID NO: 47, the CDR2 region comprises an amino acid sequence of SEQ ID NO: 222, and wherein the CDR3 region comprises an amino acid sequence of SEQ ID NO: 227,
(ii) the CDR1 region comprises an amino acid sequence of SEQ ID NO: 47, the CDR2 region comprises an amino acid sequence of SEQ ID NO: 223, and wherein the CDR3 region comprises an amino acid sequence of SEQ ID NO: 49,
(jj) the CDR1 region comprises an amino acid sequence of SEQ ID NO: 213, the CDR2 region comprises an amino acid sequence of SEQ ID NO: 51, and wherein the CDR3 region comprises an amino acid sequence of SEQ ID NO: 226,
(kk) the CDR1 region comprises an amino acid sequence of SEQ ID NO: 214, the CDR2 region comprises an amino acid sequence of SEQ ID NO: 48, and wherein the CDR3 region comprises an amino acid sequence of SEQ ID NO: 226,
(ll) the CDR1 region comprises an amino acid sequence of SEQ ID NO: 212, the CDR2 region comprises an amino acid sequence of SEQ ID NO: 221, and wherein the CDR3 region comprises an amino acid sequence of SEQ ID NO: 49,
(mm) the CDR1 region comprises an amino acid sequence of SEQ ID NO: 212, the CDR2 region comprises an amino acid sequence of SEQ ID NO: 48, and wherein the CDR3 region comprises an amino acid sequence of SEQ ID NO: 49,
(nn) the CDR1 region comprises an amino acid sequence of SEQ ID NO: 212, the CDR2 region comprises an amino acid sequence of SEQ ID NO: 221, and wherein the CDR3 region comprises an amino acid sequence of SEQ ID NO: 226,
(oo) the CDR1 region comprises an amino acid sequence of SEQ ID NO: 212, the CDR2 region comprises an amino acid sequence of SEQ ID NO: 221, and wherein the CDR3 region comprises an amino acid sequence of SEQ ID NO: 49,
(pp) the CDR1 region comprises an amino acid sequence of SEQ ID NO: 54, the CDR2 region comprises an amino acid sequence of SEQ ID NO: 55, and wherein the CDR3 region comprises an amino acid sequence of SEQ ID NO: 56,
(qq) the CDR1 region comprises an amino acid sequence of SEQ ID NO: 54, the CDR2 region comprises an amino acid sequence of SEQ ID NO: 55, and wherein the CDR3 region comprises an amino acid sequence of SEQ ID NO: 229, or
(rr) the CDR1 region comprises an amino acid sequence of SEQ ID NO: 54, the CDR2 region comprises an amino acid sequence of SEQ ID NO: 224, and wherein the CDR3 region comprises an amino acid sequence of SEQ ID NO: 56.

131. The isolated antibody of claim 129, wherein the CCR8 binding domain further comprises at least one framework region selected from the group consisting of framework 1 (FR1) region, a framework 2 (FR2) region, a framework 131 (FR3) region and a framework 4 (FR4) region.

132. The isolated antibody of claim 129, wherein the CCR8 biding domain comprises an amino acid sequence with at least 80% sequence identity to any one of SEQ ID NOs: 27-30, 70-86, or 106-111.

133. The isolated antibody of claim 129, wherein the isolated antibody further comprises a Fc region.

134. The isolated antibody of claim 133, wherein the isolated antibody further comprises one or more mutations in the Fc region relative to a corresponding wild type Fc region of SEQ ID NO:
34.

135. The isolated antibody of claim 134, wherein the one or more mutations comprises:
(a) an amino acid substitution of S252,
(b) an amino acid substitution of I351, or
(c) a combination thereof, relative to the wild type Fc region of SEQ ID NO: 34, numbering according to kabat scheme.

136. The isolated antibody of claim 135, wherein the amino acid substitution of S252 is a S252D amino acid substitution.

137. The isolated antibody of claim 135, wherein the amino acid substitution of 1351 is a 1351E amino acid substitution.

138. The isolated antibody of claim 133, wherein the Fc region comprises an amino acid sequence with at least 80% sequence identity to any one of SEQ ID NOs: 34, 44, 238 or 239.

139. The isolated antibody of claim 129, wherein the isolated antibody is a single domain antibody.

140. The isolated antibody of claim 139, wherein the single domain antibody is a VHH antibody or a VNAR antibody.

141. The isolated antibody of claim 129, wherein the isolated antibody is humanized.

142. The isolated antibody of claim 129, wherein the isolated antibody specifically binds a CCR8 polypeptide or a portion thereof.

143. The isolated antibody of claim 129, wherein the isolated antibody specifically binds a human CCR8 polypeptide or a portion thereof.

144. The isolated antibody of claim 129, wherein the isolated antibody specifically binds a transmembrane domain of a CCR8 polypeptide or a portion thereof.

145. An isolated antibody that specifically binds a Chemokine receptor 8 (CCR8) polypeptide or a portion thereof, wherein the isolated antibody comprises an amino acid sequence with at least 80% sequence identity to any one of SEQ ID NOs: 31-33, 45-46, 87-103, or 186-209, wherein the sequence identity is determined by Blastp.

146. A method of treating a subject suffering from cancer, comprising: administering to the subject an effective amount of an antibody that comprises:
a Chemokine receptor 8 (CCR8) binding domain that comprises a CDR1 region, a CDR2 region, and a CDR3 region,
wherein the CDR1 region comprises an amino acid sequence with at least 80% sequence identity to any one of SEQ ID NOs: 2, 3, 11, 12, 47, 54, 57, 58, 112-119, 139-145, or 210-214,
wherein the CDR2 region comprises an amino acid sequence with at least 80% sequence identity to any one of SEQ ID NOs: 4, 5, 6, 13, 14, 15, 48, 50, 51, 55, 59, 60, 120-125, 146-151, or 216-224, and
wherein the CDR3 region comprises an amino acid sequence with at least 80% sequence identity to any one of SEQ ID NOs: 8, 9, 17, 49, 52, 53, 56, 61, 62, 63, 64, 65, 66, 67, 68, 69, 126-137, 152-162, 225-229, 243-246, wherein the sequence identity is determined by Blastp.

147. The method of claim 146, wherein the antibody specifically binds a human CCR8 polypeptide or a portion thereof.

148. The method of claim 146, wherein the antibody inhibits CCL1-induced activation of a CCR8 polypeptide.


SEQUENCE LISTING (255 sequences)

SEQ ID NO: 1 - skipped

SEQ ID NO: 2 - protein, 8 aa, synthetic construct
GSTSSIRL

SEQ ID NO: 3 - protein, 8 aa, synthetic construct
ESTSSIRR

SEQ ID NO: 4 - protein, 7 aa, synthetic construct
SSDGSTN

SEQ ID NO: 5 - protein, 7 aa, synthetic construct
SRGGGTN

SEQ ID NO: 6 - protein, 7 aa, synthetic construct
SSGGGTN

SEQ ID NO: 7 - skipped

SEQ ID NO: 8 - protein, 13 aa, synthetic construct
GARRLLGTSRVDY

SEQ ID NO: 9 - protein, 13 aa, synthetic construct
SARRLRGTSRVDN

SEQ ID NO: 10 - skipped

SEQ ID NO: 11 - protein, 8 aa, synthetic construct
GSTTTINR

SEQ ID NO: 12 - protein, 8 aa, synthetic construct
GSTSTSNR

SEQ ID NO: 13 - protein, 7 aa, synthetic construct
SSRTSPN

SEQ ID NO: 14 - protein, 7 aa, synthetic construct
SSRTGPN

SEQ ID NO: 15 - protein, 7 aa, synthetic construct
SARTGPN

SEQ ID NO: 16 - skipped

SEQ ID NO: 17 - protein, 13 aa, synthetic construct
QARIAMGSSQVVY

SEQ ID NO: 18-19 - skipped (2 entries)

SEQ ID NO: 20 - protein, 18 aa, synthetic construct
IGWYRQAPGKQRELVARF

SEQ ID NO: 21 - protein, 37 aa, synthetic construct
YADFVKGRFTISGDNAKNTMYLQMNSLKPEDTAVYYC

SEQ ID NO: 22-23 - skipped (2 entries)

SEQ ID NO: 24 - protein, 18 aa, synthetic construct
FAWYRQAPGKQRELVAAT

SEQ ID NO: 25 - protein, 37 aa, synthetic construct
YADSVKGRFRFSRDNARSAVYLQMDNLRPEDTAVYFC

SEQ ID NO: 26 - skipped

SEQ ID NO: 27 - protein, 119 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCSARRLLGTSRVDYWGQGTQVTVSS

SEQ ID NO: 28 - protein, 119 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGSSQVVYWGLGTQVTVSS

SEQ ID NO: 29 - protein, 119 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCGARRLLGTSRVDYWGQGTQVTVSS

SEQ ID NO: 30 - protein, 119 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGASQVVYWGLGTQVTVSS

SEQ ID NO: 31 - protein, 346 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCSARRLLGTSRVDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 346 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCGARRLLGTSRVDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 346 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGSSQVVYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 444 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 219 aa, synthetic construct
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 38 - protein, 23 aa, Homo sapiens
MDYTLDLSVTTVTDYYYPDIFSS

SEQ ID NO: 39 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 40 - protein, 22 aa, Homo sapiens
METPNTTEDYDTTTEFDYGDAT

SEQ ID NO: 41 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 42 - protein, 360 aa, synthetic construct
METPNTTEDYDTTTEFDYGDATPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 43 - protein, 355 aa, synthetic construct; identical to SEQ ID NO: 39

SEQ ID NO: 44 - protein, 227 aa, synthetic construct
DKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 346 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCSARRLLGTSRVDYWGQGTQVTVSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 346 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGSSQVVYWGLGTQVTVSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 8 aa, synthetic construct
GSTSSIRR

SEQ ID NO: 48 - protein, 7 aa, synthetic construct
FSSGGGT

SEQ ID NO: 49 - protein, 13 aa, synthetic construct
SARRLLGTSRVDY

SEQ ID NO: 50 - protein, 7 aa, synthetic construct
FTSGGGT

SEQ ID NO: 51 - protein, 7 aa, synthetic construct
FSSGGST

SEQ ID NO: 52 - protein, 13 aa, synthetic construct
NARRLLGTSRVDY

SEQ ID NO: 53 - protein, 13 aa, synthetic construct
SARRLLGTSRYDY

SEQ ID NO: 54 - protein, 8 aa, synthetic construct
GSTSTINR

SEQ ID NO: 55 - protein, 7 aa, synthetic construct
TSSRTSP

SEQ ID NO: 56 - protein, 13 aa, synthetic construct
QARMAMGSSQVVY

SEQ ID NO: 57 - protein, 8 aa, synthetic construct
GSTSTINA

SEQ ID NO: 58 - protein, 8 aa, synthetic construct
GSTSSINR

SEQ ID NO: 59 - protein, 7 aa, synthetic construct
TSSGTSP

SEQ ID NO: 60 - protein, 7 aa, synthetic construct
TSSRGSP

SEQ ID NO: 61 - protein, 13 aa, synthetic construct
NARMAMGSSQVVY

SEQ ID NO: 62 - protein, 13 aa, synthetic construct
QARLAMGSSQVVY

SEQ ID NO: 63 - protein, 13 aa, synthetic construct
QARRAMGSSQVVY

SEQ ID NO: 64 - protein, 13 aa, synthetic construct
QARMTMGSSQVVY

SEQ ID NO: 65 - protein, 13 aa, synthetic construct
QARMAMGSSWVVY

SEQ ID NO: 66 - protein, 13 aa, synthetic construct
QARMAMGSSRVVY

SEQ ID NO: 67 - protein, 13 aa, synthetic construct
QARMAMGVSQVVY

SEQ ID NO: 68 - protein, 13 aa, synthetic construct
QARMAMGSSQVDY

SEQ ID NO: 69 - protein, 13 aa, synthetic construct
QARMAMASSQVVY

SEQ ID NO: 70 - protein, 119 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCNARRLLGTSRVDYWGQGTQVTVSS

SEQ ID NO: 71 - protein, 119 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCSARRLLGTSRYDYWGQGTQVTVSS

SEQ ID NO: 72 - protein, 119 aa, synthetic construct
QVQLQESGGGLVQAGGSLRLSCVASGSTSSIRRIGWYRQAPGKQRELVARFTSGGGTNYADFVKGRFTISGDNAKNTMYLQMNSLKPEDTAVYYCNARRLLGTSRVDYWGQGTQVTVSS

SEQ ID NO: 73 - protein, 119 aa, synthetic construct
QVQLQESGGGLVQAGGSLRLSCVASGSTSSIRRIGWYRQAPGKQRELVARFSSGGSTNYADFVKGRFTISGDNAKNTMYLQMNSLKPEDTAVYYCSARRLLGLSRVDYWGQGTQVTVSS

SEQ ID NO: 74 - protein, 119 aa, synthetic construct
QVQLQESGGGLVQAGGSLRLSCVASGSTSSIRRIGWYRQAPGKQRELVARFSSGGSTNYADFVKGRFTISGDNAKNTMYLQMNSLKPEDTAVYYCSARRLLGTSRYDYWGQGTQVTVSS

SEQ ID NO: 75 - protein, 119 aa, synthetic construct
QVQLQESGGGLVQAGGSLRLSCVASGSTSSIRRIGWYRQAPGKQRELVARFSSGGSTNYADFVKGRFTISGDNAKNTMYLQMNSLKPEDTAVYYCSARRLLGTSRVNYWGQGTQVTVSS

SEQ ID NO: 76 - protein, 119 aa, synthetic construct
QVQLQESGGGLVQAGGSLRLSCVASGSTSSIRRIGWYRQAPGKQRELVARFTSGGGTNYADFVKGRFTISGDNAKNTMYLQMNSLKPEDTAVYYCSARRLLGTSRVGYWGQGTQVTVSS

SEQ ID NO: 77 - protein, 119 aa, synthetic construct
QVQLQESGGGLVQAGGSLRLSCVASGSTSSIRRIGWYRQAPGKQRELVARFTSGGGTNYADFVKGRFTISGDNAKNTMYLQMNSLKPEDTAVYYCSARRLLGTSRVDSWGQGTQVTVSS

SEQ ID NO: 78 - protein, 119 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCNARMAMGSSQVVYWGLGTQVTVSS

SEQ ID NO: 79 - protein, 119 aa, synthetic construct
QVQLQESGGGLVQAGGSLRLTCAVSGSTSTINRFAWYRQAPGKQRELVAATSSGTSPNYADSVKGRFRFSRDNARSAVYLQMDNLRPEDTAVYFCQARLAMGSSQVVYWGLGTQVTVSS

SEQ ID NO: 80 - protein, 119 aa, synthetic construct
QVQLQESGGGLVQAGGSLRLTCAVSGSTSTINRFAWYRQAPGKQRELVAATSSRGSPNYADSVKGRFRFSRDNARSAVYLQMDNLRPEDTAVYFCQARRAMGSSQVVYWGLGTQVTVSS

SEQ ID NO: 81 - protein, 119 aa, synthetic construct
QVQLQESGGGLVQAGGSLRLTCAVSGSTSTINRFAWYRQAPGKQRELVAATSSRGSPNYADSVKGRFRFSRDNARSAVYLQMDNLRPEDTAVYFCQARMTMGSSQVVYWGLGTQVTVSS

SEQ ID NO: 82 - protein, 119 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGSSWVVYWGLGTQVTVSS

SEQ ID NO: 83 - protein, 119 aa, synthetic construct
QVQLQESGGGLVQAGGSLRLTCAVSGSTSTINAFAWYRQAPGKQRELVAATSSRTSPNYADSVKGRFRFSRDNARSAVYLQMDNLRPEDTAVYFCQARMAMGSSRVVYWGLGTQVTVSS

SEQ ID NO: 84 - protein, 119 aa, synthetic construct
QVQLQESGGGLVQAGGSLRLTCAVSGSTSSINRFAWYRQAPGKQRELVAATSSRTSPNYADSVKGRFRFSRDNARSAVYLQMDNLRPEDTAVYFCQARMAMGVSQVVYWGLGTQVTVSS

SEQ ID NO: 85 - protein, 119 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGSSQVDYWGLGTQVTVSS

SEQ ID NO: 86 - protein, 119 aa, synthetic construct
QVQLQESGGGLVQAGGSLRLTCAVSGSTSTINRFAWYRQAPGKQRELVAATSSRGSPNYADSVKGRFRFSRDNARSAVYLQMDNLRPEDTAVYFCQARMAMASSQVVYWGLGTQVTVSS

SEQ ID NO: 87 - protein, 346 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCNARRLLGTSRVDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 346 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCSARRLLGTSRYDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 346 aa, synthetic construct
QVQLQESGGGLVQAGGSLRLSCVASGSTSSIRRIGWYRQAPGKQRELVARFTSGGGTNYADFVKGRFTISGDNAKNTMYLQMNSLKPEDTAVYYCNARRLLGTSRVDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 346 aa, synthetic construct
QVQLQESGGGLVQAGGSLRLSCVASGSTSSIRRIGWYRQAPGKQRELVARFSSGGSTNYADFVKGRFTISGDNAKNTMYLQMNSLKPEDTAVYYCSARRLLGLSRVDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 346 aa, synthetic construct
QVQLQESGGGLVQAGGSLRLSCVASGSTSSIRRIGWYRQAPGKQRELVARFSSGGSTNYADFVKGRFTISGDNAKNTMYLQMNSLKPEDTAVYYCSARRLLGTSRYDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 346 aa, synthetic construct
QVQLQESGGGLVQAGGSLRLSCVASGSTSSIRRIGWYRQAPGKQRELVARFSSGGSTNYADFVKGRFTISGDNAKNTMYLQMNSLKPEDTAVYYCSARRLLGTSRVNYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 346 aa, synthetic construct
QVQLQESGGGLVQAGGSLRLSCVASGSTSSIRRIGWYRQAPGKQRELVARFTSGGGTNYADFVKGRFTISGDNAKNTMYLQMNSLKPEDTAVYYCSARRLLGTSRVGYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 346 aa, synthetic construct
QVQLQESGGGLVQAGGSLRLSCVASGSTSSIRRIGWYRQAPGKQRELVARFTSGGGTNYADFVKGRFTISGDNAKNTMYLQMNSLKPEDTAVYYCSARRLLGTSRVDSWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 346 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCNARMAMGSSQVVYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 346 aa, synthetic construct
QVQLQESGGGLVQAGGSLRLTCAVSGSTSTINRFAWYRQAPGKQRELVAATSSGTSPNYADSVKGRFRFSRDNARSAVYLQMDNLRPEDTAVYFCQARLAMGSSQVVYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 346 aa, synthetic construct
QVQLQESGGGLVQAGGSLRLTCAVSGSTSTINRFAWYRQAPGKQRELVAATSSRGSPNYADSVKGRFRFSRDNARSAVYLQMDNLRPEDTAVYFCQARRAMGSSQVVYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 346 aa, synthetic construct
QVQLQESGGGLVQAGGSLRLTCAVSGSTSTINRFAWYRQAPGKQRELVAATSSRGSPNYADSVKGRFRFSRDNARSAVYLQMDNLRPEDTAVYFCQARMTMGSSQVVYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 346 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGSSWVVYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 346 aa, synthetic construct
QVQLQESGGGLVQAGGSLRLTCAVSGSTSTINAFAWYRQAPGKQRELVAATSSRTSPNYADSVKGRFRFSRDNARSAVYLQMDNLRPEDTAVYFCQARMAMGSSRVVYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 346 aa, synthetic construct
QVQLQESGGGLVQAGGSLRLTCAVSGSTSSINRFAWYRQAPGKQRELVAATSSRTSPNYADSVKGRFRFSRDNARSAVYLQMDNLRPEDTAVYFCQARMAMGVSQVVYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 346 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGSSQVDYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 346 aa, synthetic construct
QVQLQESGGGLVQAGGSLRLTCAVSGSTSTINRFAWYRQAPGKQRELVAATSSRGSPNYADSVKGRFRFSRDNARSAVYLQMDNLRPEDTAVYFCQARMAMASSQVVYWGLGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 98 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 105 - protein, 20 aa, Homo sapiens
YYYYYGMDVWGQGTTVTVSS

SEQ ID NO: 106 - protein, 119 aa, synthetic construct
EVQLVESGGGLVQPGGSLRLSCAASGSTSSIRRIGWVRQAPGKGLEWVSRFSSGGGTNYADSVKGRFTISRDNSKNTLYLQMNSLRAEDTAVYYCAKRRLLGTSRVDYWGQGTTVTVSS

SEQ ID NO: 107 - protein, 119 aa, synthetic construct
EVQLVESGGGLVQPGGSLRLSCAASGSTSSIRRIGWYRQAPGKGLEWVARFSSGGGTNYADSVKGRFTISGDNSKNTMYLQMNSLRAEDTAVYYCSARRLLGTSRVDYWGQGTTVTVSS

SEQ ID NO: 108 - protein, 119 aa, synthetic construct
EVQLVESGGGLVQPGGSLRLSCAASGSTSSIRRIGWYRQAPGKGRELVARFSSGGGTNYADSVKGRFTISGDNSKNTMYLQMNSLRAEDTAVYYCSARRLLGTSRVDYWGQGTTVTVSS

SEQ ID NO: 109 - protein, 119 aa, synthetic construct
EVQLVESGGGLVQPGGSLRLSCAASGSTSTINRFAWVRQAPGKGLEWVSATSSRTSPNYADSVKGRFTISRDNSKNTLYLQMNSLRAEDTAVYYCAKRMAMGSSQVVYWGQGTTVTVSS

SEQ ID NO: 110 - protein, 119 aa, synthetic construct
EVQLVESGGGLVQPGGSLRLSCAVSGSTSTINRFAWYRQAPGKGLEWVAATSSRTSPNYADSVKGRFTFSRDNSKNTVYLQMNSLRAEDTAVYYCQARMAMGSSQVVYWGQGTTVTVSS

SEQ ID NO: 111 - protein, 119 aa, synthetic construct
EVQLVESGGGLVQPGGSLRLSCAVSGSTSTINRFAWYRQAPGKGRELVAATSSRTSPNYADSVKGRFTFSRDNSKNTVYLQMNSLRAEDTAVYYCQARMAMGSSQVVYWGQGTTVTVSS

SEQ ID NO: 112 - protein, 8 aa, synthetic construct
ASTSSIRR

SEQ ID NO: 113 - protein, 8 aa, synthetic construct
GATSSIRR

SEQ ID NO: 114 - protein, 8 aa, synthetic construct
GSASSIRR

SEQ ID NO: 115 - protein, 8 aa, synthetic construct
GSTASIRR

SEQ ID NO: 116 - protein, 8 aa, synthetic construct
GSTSAIRR

SEQ ID NO: 117 - protein, 8 aa, synthetic construct
GSTSSARR

SEQ ID NO: 118 - protein, 8 aa, synthetic construct
GSTSSIAR

SEQ ID NO: 119 - protein, 8 aa, synthetic construct
GSTSSIRA

SEQ ID NO: 120 - protein, 7 aa, synthetic construct
FASGGGT

SEQ ID NO: 121 - protein, 7 aa, synthetic construct
FSAGGGT

SEQ ID NO: 122 - protein, 7 aa, synthetic construct
FSSAGGT

SEQ ID NO: 123 - protein, 7 aa, synthetic construct
FSSGAGT

SEQ ID NO: 124 - protein, 7 aa, synthetic construct
FSSGGAT

SEQ ID NO: 125 - protein, 7 aa, synthetic construct
FSSGGGA

SEQ ID NO: 126 - protein, 13 aa, synthetic construct
AARRLLGTSRVDY

SEQ ID NO: 127 - protein, 13 aa, synthetic construct
SAARLLGTSRVDY

SEQ ID NO: 128 - protein, 13 aa, synthetic construct
SARALLGTSRVDY

SEQ ID NO: 129 - protein, 13 aa, synthetic construct
SARRALGTSRVDY

SEQ ID NO: 130 - protein, 13 aa, synthetic construct
SARRLAGTSRVDY

SEQ ID NO: 131 - protein, 13 aa, synthetic construct
SARRLLATSRVDY

SEQ ID NO: 132 - protein, 13 aa, synthetic construct
SARRLLGASRVDY

SEQ ID NO: 133 - protein, 13 aa, synthetic construct
SARRLLGTARVDY

SEQ ID NO: 134 - protein, 13 aa, synthetic construct
SARRLLGTSAVDY

SEQ ID NO: 135 - protein, 13 aa, synthetic construct
SARRLLGTSRADY

SEQ ID NO: 136 - protein, 13 aa, synthetic construct
SARRLLGTSRVAY

SEQ ID NO: 137 - protein, 13 aa, synthetic construct
SARRLLGTSRVDA

SEQ ID NO: 138 - protein, 8 aa, synthetic construct
ASTSTINR

SEQ ID NO: 139 - protein, 8 aa, synthetic construct
GATSTINR

SEQ ID NO: 140 - protein, 8 aa, synthetic construct
GSASTINR

SEQ ID NO: 141 - protein, 8 aa, synthetic construct
GSTATINR

SEQ ID NO: 142 - protein, 8 aa, synthetic construct
GSTSAINR

SEQ ID NO: 143 - protein, 8 aa, synthetic construct
GSTSTANR

SEQ ID NO: 144 - protein, 8 aa, synthetic construct
GSTSTIAR

SEQ ID NO: 145 - protein, 8 aa, synthetic construct
GSTSTINA

SEQ ID NO: 146 - protein, 7 aa, synthetic construct
TASRTSP

SEQ ID NO: 147 - protein, 7 aa, synthetic construct
TSARTSP

SEQ ID NO: 148 - protein, 7 aa, synthetic construct
TSSATSP

SEQ ID NO: 149 - protein, 7 aa, synthetic construct
TSSRASP

SEQ ID NO: 150 - protein, 7 aa, synthetic construct
TSSRTAP

SEQ ID NO: 151 - protein, 7 aa, synthetic construct
TSSRTAA

SEQ ID NO: 152 - protein, 13 aa, synthetic construct
AARMAMGSSQVVY

SEQ ID NO: 153 - protein, 13 aa, synthetic construct
QAAMAMGSSQVVY

SEQ ID NO: 154 - protein, 13 aa, synthetic construct
QARAAMGSSQVVY

SEQ ID NO: 155 - protein, 13 aa, synthetic construct
QARMAAGSSQVVY

SEQ ID NO: 156 - protein, 13 aa, synthetic construct
QARMAMASSQVVY

SEQ ID NO: 157 - protein, 13 aa, synthetic construct
QARMAMGASQVVY

SEQ ID NO: 158 - protein, 13 aa, synthetic construct
QARMAMGSAQVVY

SEQ ID NO: 159 - protein, 13 aa, synthetic construct
QARMAMGSSAVVY

SEQ ID NO: 160 - protein, 13 aa, synthetic construct
QARMAMGSSQAVY

SEQ ID NO: 161 - protein, 13 aa, synthetic construct
QARMAMGSSQVAY

SEQ ID NO: 162 - protein, 13 aa, synthetic construct
QARMAMGSSQVVA

SEQ ID NO: 163 - protein, 25 aa, synthetic construct
QVQLQESGGGLVQAGGSLRLSCVAS

SEQ ID NO: 164 - protein, 25 aa, synthetic construct [sequence omitted]

SEQ ID NO: 165 - protein, 17 aa, synthetic construct
IGWYRQAPGKQRELVAR

SEQ ID NO: 166 - protein, 17 aa, synthetic construct
IGWVRQAPGKGLEWVSR

SEQ ID NO: 167 - protein, 17 aa, synthetic construct
IGWYRQAPGKGLEWVAR

SEQ ID NO: 168 - protein, 17 aa, synthetic construct
IGWYRQAPGKGRELVAR

SEQ ID NO: 169 - protein, 38 aa, synthetic construct
NYADFVKGRFTISGDNAKNTMYLQMNSLKPEDTAVYYC

SEQ ID NO: 170 - protein, 38 aa, synthetic construct
NYADSVKGRFTISRDNSKNTLYLQMNSLRAEDTAVYYC

SEQ ID NO: 171 - protein, 38 aa, synthetic construct
NYADSVKGRFTISGDNSKNTMYLQMNSLRAEDTAVYYC

SEQ ID NO: 172 - protein, 11 aa, synthetic construct
WGQGTQVTVSS

SEQ ID NO: 173 - protein, 11 aa, synthetic construct
WGQGTTVTVSS

SEQ ID NO: 174 - skipped

SEQ ID NO: 175 - protein, 25 aa, synthetic construct
QVQLQESGGGLVQAGGSLRLTCAVS

SEQ ID NO: 176 - protein, 25 aa, synthetic construct [sequence omitted]

SEQ ID NO: 177 - protein, 25 aa, synthetic construct [sequence omitted]

SEQ ID NO: 178 - protein, 17 aa, synthetic construct
FAWYRQAPGKQRELVAA

SEQ ID NO: 179 - protein, 17 aa, synthetic construct
FAWVRQAPGKGLEWVSA

SEQ ID NO: 180 - protein, 17 aa, synthetic construct
FAWYRQAPGKGLEWVAA

SEQ ID NO: 181 - protein, 17 aa, synthetic construct
FAWYRQAPGKGRELVAA

SEQ ID NO: 182 - protein, 38 aa, synthetic construct
NYADSVKGRFRFSRDNARSAVYLQMDNLRPEDTAVYFC

SEQ ID NO: 183 - protein, 38 aa, synthetic construct; identical to SEQ ID NO: 170

SEQ ID NO: 184 - protein, 38 aa, synthetic construct
NYADSVKGRFTFSRDNSKNTVYLQMNSLRAEDTAVYYC

SEQ ID NO: 185 - protein, 11 aa, synthetic construct
WGLGTQVTVSS

SEQ ID NO: 186 - protein, 119 aa, synthetic construct
EVQLVESGGGLVQPGGSLRLSCAASGSTSSIRRIGWYRQAPGKGRELVARFSSGGSTNYADSVKGRFTISGDNSKNTMYLQMNSLRAEDTAVYYCSARRLLGTSRVDYWGQGTTVTVSS

SEQ ID NO: 187 - protein, 119 aa, synthetic construct
EVQLVESGGGLVQPGGSLRLSCAASGSTSSIRRIGWYRQAPGKGRELVARFSSGGGTNYADSVKGRFTISGDNSKNTMYLQMNSLRAEDTAVYYCSARRLVGTSRVDYWGQGTTVTVSS

SEQ ID NO: 188 - protein, 119 aa, synthetic construct
EVQLVESGGGLVQPGGSLRLSCAASGSTSSIRRYSWYRQAPGKGRELVAWFSSGGGTNYADSVKGRFTISGDNSKNTMYLQMNSLRAEDTAVYYCSARRLLGTSRVDYWGQGTQVTVSS

SEQ ID NO: 189 - protein, 119 aa, synthetic construct
EVQLVESGGGLVQPGGSLRLSCAASGSTSSIRTIGWYRQAPGKGRELVARFSSGGGTNYADSVKGRFTISGDNSKNTMYLQMNSLRAEDTAVYYCSARILLGTSRVDYWGQGTTVTVSS

SEQ ID NO: 190 - protein, 119 aa, synthetic construct
EVQLVESGGGLVQPGGSLRLSCAASGSTSSIRRIGWYRQAPGKGRELVAGFSSGGGTNYADSVKGRFTISGDNSKNTMYLQMNSLRAEDTAVYYCSARRLLGTSRVDYWGQGTTVTVSS

SEQ ID NO: 191 - protein, 119 aa, synthetic construct
EVQLVESGGGLVQPGGSLRLSCAASGSTSSIRRIGWYRQAPGKGRELVAHFSSGGGTNYADSVKGRFTISGDNSKNTMYLQMNSLRAEDTAVYYCSARRLLGTSRVDYWGQGTTVTVSS

SEQ ID NO: 192 - protein, 119 aa, synthetic construct
EVQLVESGGGLVQPGGSLRLSCAVSGSTSSIRRIGWYRQAPGKGRELVAHFSSGGGTNYADSVKGRFTISGDKSKNTMYLQMNSLRAEDTAVYYCSARRLLGTSRVDYWGQGTTVTVSS

SEQ ID NO: 193 - protein, 119 aa, synthetic construct
EVQLVESGGGLVQPGGSLRLSCAASGSTSSIRRIGWYRQAPGKGRELVARFSSGIGTNYADSVKGRFTISGDNSKNTMYLQMNSLRAEDTAVYYCSARRLVGTSRVDYWGQGTQVTVSS

SEQ ID NO: 194 - protein, 119 aa, synthetic construct
EVQLVESGGGLVQPGGSLRLSCAASGSTSSIRRIGWYRQAPGKGRELVARFSGGGSINYADSVKGRFTISGDNSKNTMYLQMNSLRAEDTAVYYCSARRLVGTSRVDYWGQGTQVTVSS

SEQ ID NO: 195 - protein, 119 aa, synthetic construct
QVQLQESGGGLVQPGGSLRLSCAASGSTSSIRRIGWYRQAPGKGRELVARFSSGGGINYADSVKGRFTISGDNSKNTMYLQMNSLRAEDTAVYYCSARRLVGTSRVDYWGQGTQVTVSS

SEQ ID NO: 196 - protein, 119 aa, synthetic construct
QVQLQESGGGLVQPGGSLRLSCAASGSTSSIRRIGWYRQAPGKGRELVARFSSGGTINYADSVKGRFTISGDNSKNTMYLQMNSLRAEDTAVYYCSARRLLGTSRVDYWGQGTQVTVSS

SEQ ID NO: 197 - protein, 119 aa, synthetic construct
EVQLVESGGGLVQPGGSLRLSCAASGFTSSIRRIGWYRQAPGKGRELVARFSSGGTTNYADSVKGRFTISGDNSKNTMYLQMNSLRAEDTAVYYCSARRLYGTSRVDYWGQGTQVTVSS

SEQ ID NO: 198 - protein, 119 aa, synthetic construct
EVQLVESGGGLVQPGGSLRLSCAVSGSTSSIRGIGWYRQAPGKGRELVARFISGGGTNYADSVKGRFTISGDNSKNTMYLQMNSLRAEDTAVYYCSARRLLGTSRVDYWGQGTQVTVSS

SEQ ID NO: 199 - protein, 119 aa, synthetic construct
EVQLVESGGGLVQPGGSLRLSCAASGSTSSIRRIGWYRQAPGKGRELVARFSSGGSINYADSVKGRFTISGDNSKNTMYLQMNSLRAEDTAVYYCSARRLVGTSRVDYWGQGTQVTVSS

SEQ ID NO: 200 - protein, 119 aa, synthetic construct
EVQLVESGGGLVQPGGSLRLSCAASGSTSSIRRIGWYRQAPGKGRELVARFSGGGGTNYADSVKGRFTISGDNSKNTMYLQMNSLRAEDTAVYYCSARRLLGTSRVDYWGQGTTVTVSS

SEQ ID NO: 201 - protein, 119 aa, synthetic construct
EVQLVESGGGLVQPGGSLRLSCAASGSTSTIVRIGWYRQAPGKGRELVARFSSGGSTNYADSVKGRFTISGDNSKNTMYLQMNSLRAEDTAVYYCSARILLGTSRVDYWGQGTTVTVSS

SEQ ID NO: 202 - protein, 119 aa, synthetic construct
EVQLVESGGGLVQPGGSLRLSCAVSGSTSSIVRIGWYRQAPGKGRELVAHFSSGGGTNYADSVKGRFTISGDKSKNTMYLQMNSLRAEDTAVYYCSARILLGTSRVDYWGQGTTVTVSS

SEQ ID NO: 203 - protein, 119 aa, synthetic construct
EVQLVESGGGLVQPGGSLRLSCAVSGSTSSIRGIGWYRQAPGKGRELVARFISGGGTNYADSVKGRFTISGDKSKNTMYLQMNSLRAEDTAVYYCSARRLLGTSRVDYWGQGTTVTVSS

SEQ ID NO: 204 - protein, 119 aa, synthetic construct
EVQLVESGGGLVQPGGSLRLSCAVSGSTSSIRGIGWYRQAPGKGRELVAHFSSGGGTNYADSVKGRFTISGDKSKNTMYLQMNSLRAEDTAVYYCSARRLLGTSRVDYWGQGTQVTVSS

SEQ ID NO: 205 - protein, 119 aa, synthetic construct
EVQLVESGGGLVQPGGSLRLSCAVSGSTSSIRGIGWYRQAPGKGRELVARFISGGGTNYADSVKGRFTISGDNSKNTMYLQMNSLRAEDTAVYYCSARILLGTSRVDYWGQGTQVTVSS

SEQ ID NO: 206 - protein, 119 aa, synthetic construct
EVQLVESGGGLVQPGGSLRLSCAVSGSTSSIRGIGWYRQAPGKGRELVARFISGGGTNYADSVKGRFTISADNSKNTMYLQMNSLRAEDTAVYYCSARRLLGTSRVDYWGQGTQVTVSS

SEQ ID NO: 207 - protein, 119 aa, synthetic construct
EVQLVESGGGLVQPGGSLRLSCAVSGSTSTINRFAWYRQAPGKGRELVAATSSRTSPNYADSVKGRFTFSRGNSKNTVYLQMNSLRAEDTAVYYCQARMAMGSSQVVYWGQGTTVTVSS

SEQ ID NO: 208 - protein, 119 aa, synthetic construct
EVQLVESGGGLVQPGGSLRLSCAVSGSTSTINRFAWYRQAPGKGRELVAATSSRTSPNYADSVKGRFTFSRGNSKNTVYLQMNSLRAEDTAVYYCQARKAMGSSQVVYWGQGTTVTVSS

SEQ ID NO: 209 - protein, 119 aa, synthetic construct
EVQLVESGGGLVQPGGSLRLSCAVSGSTSTINRFAWYRQAPGKGRELVAATSSRTSLNYADSVKGRFTFSRDNSKNTVYLQMNSLRAEDTAVYYCQARMAMGSSQVVYWGQGTTVTVSS

SEQ ID NO: 210 - protein, 8 aa, synthetic construct
GSTSSIRT

SEQ ID NO: 211 - protein, 8 aa, synthetic construct
GFTSSIRR

SEQ ID NO: 212 - protein, 8 aa, synthetic construct
GSTSSIRG

SEQ ID NO: 213 - protein, 8 aa, synthetic construct
GSTSTIVR

SEQ ID NO: 214 - protein, 8 aa, synthetic construct
GSTSSIVR

SEQ ID NO: 215 - skipped

SEQ ID NO: 216 - protein, 7 aa, synthetic construct
FSSGIGT

SEQ ID NO: 217 - protein, 7 aa, synthetic construct
FSGGGSI

SEQ ID NO: 218 - protein, 7 aa, synthetic construct
FSSGGGI

SEQ ID NO: 219 - protein, 7 aa, synthetic construct
FSSGGTI

SEQ ID NO: 220 - protein, 7 aa, synthetic construct
FSSGGTT

SEQ ID NO: 221 - protein, 7 aa, synthetic construct
FISGGGT

SEQ ID NO: 222 - protein, 7 aa, synthetic construct
FSSGGSI

SEQ ID NO: 223 - protein, 7 aa, synthetic construct
FSGGGGT

SEQ ID NO: 224 - protein, 7 aa, synthetic construct
TSSRTSL

SEQ ID NO: 225 - skipped

SEQ ID NO: 226 - protein, 13 aa, synthetic construct
SARILLGTSRVDY

SEQ ID NO: 227 - protein, 13 aa, synthetic construct
SARRLVGTSRVDY

SEQ ID NO: 228 - protein, 13 aa, synthetic construct
SARRLYGTSRVDY

SEQ ID NO: 229 - protein, 13 aa, synthetic construct
QARKAMGSSQVVY

SEQ ID NO: 230-231 - skipped (2 entries)

SEQ ID NO: 232 - protein, 17 aa, synthetic construct
IGWYRQAPGKGRELVAG

SEQ ID NO: 233 - protein, 17 aa, synthetic construct
IGWYRQAPGKGRELVAH

SEQ ID NO: 234 - protein, 38 aa, synthetic construct
NYADSVKGRFTISGDKSKNTMYLQMNSLRAEDTAVYYC

SEQ ID NO: 235 - protein, 38 aa, synthetic construct
NYADSVKGRFTISADNSKNTMYLQMNSLRAEDTAVYYC

SEQ ID NO: 236 - protein, 38 aa, synthetic construct
NYADSVKGRFTFSRGNSKNTVYLQMNSLRAEDTAVYYC

SEQ ID NO: 237 - skipped

SEQ ID NO: 238 - protein, 233 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 239 - protein, 352 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCSARRLLGTSRVDYWGQGTQVTVSSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 240 - protein, 444 aa, synthetic construct
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 241-242 - skipped (2 entries)

SEQ ID NO: 243 - protein, 13 aa, synthetic construct
SARRLLGLSRVDY

SEQ ID NO: 244 - protein, 13 aa, synthetic construct
SARRLLGTSRVNY

SEQ ID NO: 245 - protein, 13 aa, synthetic construct
SARRLLGTSRVGY

SEQ ID NO: 246 - protein, 13 aa, synthetic construct
SARRLLGTSRVDS

SEQ ID NO: 247 - protein, 4 aa, synthetic construct
GGFG

SEQ ID NO: 248 - protein, 4 aa, synthetic construct
ALAL

SEQ ID NO: 249 - protein, 4 aa, synthetic construct
GFLG

SEQ ID NO: 250 - protein, 6 aa, synthetic construct
HHHHHH

SEQ ID NO: 251 - protein, 115 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCSARRLLGTSRVDYWGQGTQV

SEQ ID NO: 252 - protein, 115 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGSSQVVYWGLGTQV

SEQ ID NO: 253 - protein, 115 aa, synthetic construct
QVQLQESGGGLVQAGGSLRLSCVASGSTSSIRRIGWYRQAPGKQRELVARFSSGGGTNYADFVKGRFTISGDNAKNTMYLQMNSLKPEDTAVYYCGARRLLGTSRVDYWGQGTQV

SEQ ID NO: 254 - protein, 115 aa, synthetic construct
QVQLQESGGGLVQAGGSLRLTCAVSGSTSTINRFAWYRQAPGKQRELVAATSSRTSPNYADSVKGRFRFSRDNARSAVYLQMDNLRPEDTAVYFCQARMAMGASQVVYWGLGTQV

SEQ ID NO: 255 - protein, 107 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCWGQGTTVTVSS